(19) United States
(12) Patent Application Publication
(10) Pub. No.: US 2010/0273865 A1
(43) Pub. Date: Oct. 28, 2010

(54) Polypeptides involved in neuronal regeneration-associated gene expression

(75) Inventors: August Benjamin Smit, Amsterdam (NL); Joost Verhaagen, Amsterdam (NL); Harold Mac Gillavry, Utrecht (NL); Ronald Ernst Van Kesteren, Almere (NL)

Correspondence Address: EDWARDS ANGELL PALMER & DODGE LLP, P.O. BOX 55874, BOSTON, MA, 02205, US

(73) Assignees: Vereniging voor Christelijk Hoger Onderwijs, Wetenschappelijk Onderzoek en Patientenzorg; Nederlands Instituut voor Neurowetenschappen

(21) Appl. No.: 12/771,417

(22) Filed: Apr. 30, 2010

Related U.S. Application Data
(63) Continuation of application No. PCT/NL08/50684, filed on Oct. 31, 2008.
(60) Provisional application No. 60/984,842, filed on Nov. 2, 2007.

(51) Int. Cl. A61K 48/00; C12N 5/16; C07H 21/04; A61P 25/28; A61P 25/16; C12Q 1/02

(52) U.S. Cl. 514/44 R; 435/375; 536/23.5; 435/29

(57) ABSTRACT

The present invention relates to methods for promoting regeneration response of peripheral and central nervous systems in mammals in need of such biological effects. The methods comprise altering the activity or steady state level of specific transcription factors that control regeneration of injured or degenerated neuronal cells. Preferably the activity or steady state level of specific transcription factors is altered by introducing nucleic acids to increase or decrease expressing of these transcription factors. These are useful in or suffering from neurodegenerative disorders.

### RELATED APPLICATIONS

This present invention is a continuation patent application that claims priority to PCT patent application number PCT/NL2008/050684, filed Oct. 31, 2008, which claims the benefit of U.S. Application 60/984,842, filed on Nov. 2, 2007, the entirety of which are herein incorporated by reference.

### FIELD OF THE INVENTION

The present invention relates to a polypeptide and to a nucleic acid encoding them, whose expression is modulated in cells of the dorsal root ganglia undergoing a regenerative response elicited by crush damage of the sciatic nerve. These nucleic acids are useful in methods for controlling a regeneration response of peripheral and central nervous systems in mammals in need of such biological effects, including the treatment of humans after neurotraumatic injury, e.g. after lesion, avulsion or contusion of nerve tissue.

### BACKGROUND OF THE INVENTION

Most spinal cord injuries in humans are caused by road traffic, work or sports accidents and involve (i) fractures or dislocations of the vertebrae resulting in contusion of the spinal cord and disruption of the major ascending and descending pathways, including the corticospinal tracts (CST), and/or (ii) avulsion of dorsal and/or ventral spinal roots thereby disconnecting the spinal cord from the peripheral nerves. Both injuries to the long tracts and local nerve root injuries have serious consequences for the patient. About 50% of all spinal cord injured patient are tetraplegic (both arms and legs are affected) and the other half is paraplegic (legs are effected, but arms not). Spinal cord injury affects mostly young, healthy individuals that are part of the workforce and lead productive lives. Most patients surviving the acute phase of spinal cord injury will become wheel chair bound and have a life expectancy of several decades. To date no effective treatments for spinal cord or spinal root injuries are available. In the case of ventral root avulsion some success has been reported with surgical reimplantation of the avulsed roots into the spinal cord. Recovery of arm and shoulder function as a result of this neurosurgical intervention is, however, very limited.

Most axons in the central nervous system (CNS) do not regenerate after injury, whereas damaged axons in the peripheral nervous system (PNS) do regrow and reinnervate target cells. Successful regeneration of peripheral neurons is in part attributed to the growth-permissive cellular environment, whereas in the CNS a growth-inhibiting environment restricts outgrowth of damaged neurons (Yiu and He, 2006). Another major contributing factor to successful axonal regeneration is the intrinsic ability of neurons to allow regrowth of injured axons (Raivich and Makwana, 2007). Dorsal root ganglion (DRG) neurons are an attractive model to study neuron-intrinsic mechanisms of regeneration. These neurons extend one axon into the spinal nerve and one axon into the dorsal root and ascending dorsal columns. The peripheral and central branches of DRG neurons differ in their capacity to regenerate: a peripheral nerve crush results in vigorous regeneration of injured axons, but after dorsal root crush regeneration of injured nerve fibres is significantly impaired (for review, see Teng and Tang, 2006). Successful regeneration of DRG neurons following peripheral axotomy is transcription-dependent (Smith and Skene, 1997), and requires retrograde transport of injury-induced signals from the lesion site to the nuclei of the injured neurons (Chong et al., 1999; Hanz et al., 2003; Neumann and Woolf, 1999). Injured DRG neurons show increased expression of many regeneration-associated genes, including growth-associated protein 43 (Gap43), cytoskeleton-associated protein 23 (Cap23) and arginase 1 (Arg1) (Cai et al., 2002; Chong et al., 1994; Frey et al., 2000; Skene et al., 1986; Verge et al., 1990; Woolf et al., 1990). Proteins encoded by these genes induce cytoskeletal rearrangements and polyamine synthesis, respectively, and stimulate axonal outgrowth when overexpressed in injured neurons (Aigner et al., 1995; Bomze et al., 2001; Cai et al., 2002; Frey et al., 2000). Injury-induced expression of regeneration-associated genes during successful regeneration probably requires the coordinated activity of regeneration-associated transcription factors (TFs). To date, several injury-responsive TFs have been identified that promote axonal outgrowth, including cAMP response element binding protein (CREB; Gao et al., 2004), signal transducer and activator of transcription-3 (STAT3; Qiu et al., 2005), activating transcription factor-3 (ATF3; Seijffers et al., 2006; Seijffers et al., 2007), the activator protein-1 (AP-1) component c-Jun (Broude et al., 1997; Raivich et al., 2004) and SRY-box containing gene-11 (Sox11; Jankowski et al., 2006).

Transcription regulatory mechanisms are revealed by the underlying gene regulatory networks describing the dynamic relationships between TF expression, TF binding and target gene expression (Goutsias and Lee, 2007). An important property of gene regulatory networks is that they are composed of smaller network motifs of co-regulated TFs which tend to be evolutionarily conserved and reoccur in functionally different networks (Alon, 2007). Cellular context determines how these network motifs interact in order to generate cell type specific transcriptional responses. In spite of all published studies, there is not yet a comprehensive view of the transcriptional regulatory mechanisms underlying neuronal regeneration.

Thus it is an object of the invention to provide for the key transcription factors and/or encoding nucleic acids in the process of neural repair. It is a further object of the invention to provide for therapies bases on these transcription factors and/or nucleic acids that promote the repair process leading to return of function in neurotrauma patients.

### BRIEF DESCRIPTION OF THE DRAWINGS

FIG. 1. High-content screening identifies TFs involved in regenerative neurite outgrowth. (A) Cellomics KineticScan HCS Reader-obtained images of F11 cells stained with anti-neurofilament showing forskolin-induced neurite outgrowth. (B) The same image as in (A), showing how the Cellomics Neuronal Profiling algorithm accurately traces neurites based on anti-neurofilament staining (C) Cellomics quantification of forskolin-induced neurite outgrowth from F11 cells showing a dose-dependent increase in neurite total length. Data points represent means±SEM; n=6 wells for each concentration of forskolin. (D) Heatmap showing the log fold change in expression of selected TFs in forskolin-stimulated F11 cells as measured by qPCR. (E) The mean regulation of selected TFs in regenerating DRG neurons (y-axis; Stam et al., 2007) and in forskolin-stimulated F11 cells (x-axis) are clearly correlated (r=0.771; p=0.001). (F, G, H) Examples of forskolin-stimulated F11 cells transfected with control siRNA (F), siATF3 (G) and siNFIL3 (H) showing reduced neurite outgrowth after knock-down of ATF3 and enhanced neurite outgrowth after knock-down of NFIL3. (I, J) Volcano plots summarizing the screening results for all 62 TFs. Values represent log normalized means in neurite total length (I) or fraction of outgrowth positive cells (J) after TF knock-down. TFs showing effects that are statistically significant (p<0.01; horizontal dotted lines) and biologically relevant (effect size>1 SD of the combined negative controls; vertical dotted lines) in both assays are indicated in red.

FIG. 2. NFIL3 expression is specifically up-regulated during successful regeneration. (A) qPCR analysis demonstrates a robust and specific up-regulation of NFIL3 mRNA after sciatic nerve crush, corroborating previously reported microarray data (Stam et al., 2007). (B) In situ hybridization confirms that NFIL3 is up-regulated in DRGs after sciatic nerve crush and shows that NFIL3 mRNA is present in most neurons of the injured DRG.

FIG. 3. NFIL3 expression in F11 cells is induced by forskolin. (A) NFIL3 mRNA expression is induced in forskolin-stimulated F11 cells as measured by qPCR. Data points represent means±SEM; n=5 for each time-point. (B) Western blot analysis demonstrates up-regulation of NFIL3 protein starting from 1 h after forskolin stimulation. Phospho-CREB (Ser133) is apparent already 30 min after stimulation. Total CREB levels are shown for comparison. (C) Confocal images of forskolin-stimulated F11 cell showing nuclear localization of NFIL3. (D) Western blot analysis of cytoplasmic and nuclear extracts of forskolin-stimulated F11 cells confirms nuclear localization of NFIL3.

FIG. 4. NFIL3 knock-down enhances neurite outgrowth from F11 cells. (A) siNFIL3 causes a reduction in NFIL3 mRNA levels. The normal forskolin-induced increase in NFIL3 mRNA levels is absent in siNFIL3-treated cells. (B) Western blotting confirms that siNFIL3 causes knock-down of NFIL3 protein in HEK293 cells overexpressing NFIL3. The siNFIL3 pool and well as two individual siRNAs (#2 and #3) significantly reduce NFIL3 protein levels; control siRNAs (siGLO and siCONTROL) do not affect NFIL3 protein levels. (C) NFIL3 knock-down causes a significant increase in forskolin-stimulated (grey bars) and unstimulated F11 cells (white bars). (D) Overexpression of NFIL3 has no effect on forskolin stimulated neurite outgrowth. Bars represent means±SD; n=4; * p<0.01.

FIG. 5. NFIL3 is a repressor of CREB-mediated gene expression in F11 cells. (A) The reporter constructs used contain either the CREB-responsive part of the rat somatostatin gene promoter (Montminy et al., 1986) or a tandem repeat of 3 EBPRE consensus sites (Ozkurt and Tetradis, 2003). Sequence comparison shows the high degree of similarity between CRE (upper sequence) and EBPRE (lower sequence) sites. (B) Forskolin induces EBPRE- and CRE-mediated transcriptional activity in F11 cells. F11 cells were transfected with either the EBPRE or the CRE reporter construct and stimulated with forskolin for indicated times. Normalized luciferase activities are plotted (means±SD; n=3 for each condition). (C) Luciferase assays showing the effects of overexpression of CREB and NFIL3 on forskolin-stimulated transcriptional activity. These data clearly show that CREB activates both CRE and EBPRE sites, whereas NFIL3 represses both sites. Bars represent means±SD; n=3 for each condition; * p<0.01.

FIG. 6. NFIL3 represses the expression of regeneration-associated genes. (A) Chromatin immunoprecipitation assay demonstrating direct binding of NFIL3 to the promoter regions of Nfil3, Arg1, Gap43, Fos and Atf3, but not Cdkn2c and Actb, using two independent antibodies against NFIL3 (C18 and V19). (B) Schematic representations of the location of EBPRE sites (small black boxes) in the Nfil3, Gap43 and Arg1 genes. (C) Gene fragments containing the predicted EBPRE sites were cloned into the pGL2-B-luciferase plasmid. Luciferase assays show that these constructs are transcriptionally active in HEK293T cells, and that transcriptional activity is repressed when NFIL3 is co-transfected. Importantly, NFIL3 did not repress luciferase activity of a peripheral myelin PO promoter-luciferase construct (not shown). Bars represent means±SD; n=3 for each condition; * p<0.01.

FIG. 7. NFIL3 regulates neurite outgrowth in primary adult DRG neurons. (A) Confocal images showing predominant nuclear localization of NFIL3 in cultured primary adult DRG neurons. (B) DRG neurons stimulated with forskolin show a 6-fold up-regulation of NFIL3 expression as measured by qPCR. Forskolin-induced up-regulation of NFIL3 mRNA is blocked by the PKA inhibitor H89. Bars represent means±SD; n=3 for each condition. (C) Primary adult DRG neurons transfected with siNFIL3 show a 50-60% knock-down of NFIL3 mRNA levels as measured by qPCR. Control siRNA had no effect on NFIL3 mRNA levels. Bars represent means±SD; n=3 for each condition; * p<0.01. (D, E) Knock-down of NFIL3 causes an increase in neurite length of primary adult DRG neurons in culture. The mean length of the longest neurite was measured for 100-150 neurons per condition. Bars represent means±SD; * p<0.01.

FIG. 8. Proposed model for the regulation of CRE- and EBPRE-mediated transcription by CREB and NFIL3 in neuronal regeneration. Elevated levels of cAMP triggered by peripheral neuronal injury activate PKA and CREB. CREB then activates regeneration-associated genes (RAGs) containing CRE/EBPRE sites, including Nfil3. NFIL3 acts as a negatively feed back regulator on CRE/EBPRE-mediated transcription, repressing regeneration-associated genes. At this moment we cannot exclude that NFIL3 in parallel regulates the expression of other regeneration-associated genes independent of CREB.

FIG. 9. Confirmation of Dharmacon SMART siRNA pool-induced effects on neurite outgrowth by individual siRNAs. Bars represent the normalized mean neurite total length. * p<0.05.

FIG. 10. This is a table giving si-RNA-induced affects of the 62 TFs on neurite outgrowth from F11 cells.

FIG. 11. This a table giving a list of the TFs of the invention that may be used for promoting neuronal regeneration.

FIG. 12. Representation of two distinct types of dominant negatives of Nfil3.

FIG. 13. Overexpression of dominant-negative NFIL3 increases neurite outgrowth from adult DRG neurons in culture. (A) Schematic representation of full-length and dominant-negative NFIL3 protein. The dominant-negative NFIL3 protein used here lacks the DNA binding domain, which is replaced by an acidic amphipathic amino acid sequence, resulting in a higher affinity for the endogenous full-length protein (see Ahn et al. 1998). (B) Immunofluorescence staining shows cytoplasmic localization of Flag-tagged dominant-negative NFIL3 (DN-NFIL3) expressed in F11 cells. (C) Western blot analysis shows specific co-immunoprecipitation of Flag-tagged dominant-negative NFIL3 with Myc-tagged NFIL3 when co-expressed in HEK293 cells. Note that CREB does not co-immunoprecipitate with dominant-negative NFIL3. (D) Overexpression of dominant-negative NFIL3 induces neurite outgrowth from adult DRG neurons in culture. Overexpression of either full-length NFIL3 or EGFP had no effect on neurite outgrowth. Bars represent means±SD; * p<0.01.

### DESCRIPTION OF THE INVENTION

Most of the prior art gene expression analyses realized so far have only provided single snapshots of the highly complex biological process of regeneration. Therefore, it is impossible to determine whether regulated genes at a particular timepoint are genes important for the initiation of the outgrowth process, play a role during axon elongation or are involved in target finding or reestablishment of sensory contacts. In order to link a gene to a part of this process, gene expression analysis should be performed in combination with or followed up by functional screening. Functional screening serves to validate or infirm array data.

In addition, the biological interpretation of gene expression data is facilitated to a great extent if a second, related but different process is analyzed in parallel. In this respect, the DRG neuron offers the unique opportunity to compare gene expression changes during a robust outgrowth response in the sciatic nerve (SN-crush) and a weak outgrowth response in the dorsal root (DR-crush). This comparison holds the advantages that the tissue samples that will be analyzed are very similar to each other, the only biological difference being the localization of the injury inflicted to the neurite. This is not the case if, for instance, gene expression in the lesioned CNS is compared to gene expression in DRG neurons. Also differential gene expression analysis allows to eliminate stress and injury related gene expression changes which could be similar in both paradigms. If genes that are regulated in a similar fashion by both injuries are excluded from further analysis, chances are that true regeneration-associated genes are enriched. Therefore, a high resolution time-course analysis of gene expression changes after DR and SN crush were used by the present inventors to reveal nucleic acids involved in successful regeneration.

The screens for intrinsic neuronal genes have been performed on primary sensory neurons of the rat DRG (see below). These neurons are uniquely suited to study successful and abortive regeneration. The cell bodies of these neurons are located in the dorsal root ganglia and these neurons possess two branches: one projecting peripherally innervating the skin, and one branch projecting centrally to the spinal cord. The peripheral branch regenerates vigorously while the central branch regenerates virtually not. By comparing changes in gene expression after a peripheral versus a central lesion we identified novel intrinsic, genes that are up-regulated or down regulated after lesion of the peripheral branch, but not after a central branch lesion. The power of this screen was not only the comparison of peripheral versus central regeneration, but also the fact that we specifically examined gene expression during the first 6 to 72 hours (5 time points) of the regenerative response. By doing so and by using used advanced target finding technology developed as a result of the human and rodent genome projects and have discovered a large set of new genes involved in the neuronal response. In particular we were able to discover the key factors that initiate the neuronal gene program that drives successful regeneration.

In one aspect the present invention relates to a method for promoting or controlling generation or regeneration of a neuronal cell. A first method for promoting or controlling generation or regeneration of a neuronal cell comprises the step of altering the activity or the steady state level of a polypeptide in the neuronal cell or in cells in the direct environment of the neuronal cell in need of (re)generation, e.g. the supporting glia cells (see also below). A polypeptide of which an activity or steady-state level is altered is preferably a polypeptide selected from the group consisting of: a BHLHB3, ETS1, TRPC3, REST, PJA2, MTF1, TCEA2, PRRXL1, TCEB1, PDLIM7, ID2, TLE3, MAPK3, ANKRD1, SOX10, HES5, SREBF1, SMAD1, RTEL1, TCFE2A, CSRP3, TSC22D3, STAT5a, Egr1 and a NFIL3. These polypeptides are further identified by preferred encoding nucleic sequences as identified in table 1. Therefore, a polypeptide of which an activity or the steady state level is altered preferably is a polypeptide that comprises an amino acid sequence that is encoded by a nucleotide sequence selected from: (a) a nucleotide sequence that has at least 60, 70, 80, 85, 90, 95, 98 or 99% sequence identity with a nucleotide sequence selected from SEQ ID NO.'s 1-45, 54, 55, 58-63 except SEQ ID NO:15 and 36; each SEQ ID NO corresponding to an encoding sequence of a polypeptide as defined in claim 1 and as identified in table 1, except ATF3 which is represented by SEQ ID NO:15 and 36; and, (b) a nucleotide sequence that encodes an amino acid sequence that has at least 60, 70, 80, 85, 90, 95, 98 or 99% amino acid identity with an amino acid sequence that is encoded by a nucleotide sequence selected from SEQ ID NO.'s 1-45, 54, 55, 58-63 except SEQ ID NO:15 and 36; each SEQ ID NO corresponding to an encoding sequence of a polypeptide as defined in claim 1 and as identified in table 1, except ATF3 which is represented by SEQ ID NO:15 and 36. A polypeptide is herein further referred to as a polypeptide of the invention, a TF polypeptide, or briefly a TF or is identified by its name or by a preferred SEQ ID NO of an encoding nucleic acid; said nucleic acid being represented by a nucleic acid sequence. A TF polypeptide of the invention preferably is a transcription factor or a modulator of gene transcription or a putative transcriptional regulator based on sequence identity, subcellular localization or domain architecture and preferably its expression level is altered at least in the early stages (and preferably also in later stages) of regeneration. A TF preferably determines whether neurons successfully regenerate (neurite outgrowth, median neurite total length and/or mean neurite total length are positively affected). A change in the activity or the steady state level of a TF result in an altered gene expression state that is required for robust neurite outgrowth and functional recovery. Preferably a TF of the invention is thus a key switch that determines whether a damaged neuron regenerates successfully or not.

An “alteration of the activity or steady state level of a polypeptide” is herein understood to mean any detectable change in a biological activity exerted by a polypeptide or in the steady state level of a polypeptide as compared said activity or steady-state in a individual who has not been treated. All methods of the invention may be applied in any animal. Preferably, the animal is a mammal. More preferably the mammal is a human being.

The alteration of the amount of a nucleotide sequence is preferably assessed using classical molecular biology techniques such as (real time) PCR, arrays or Northern analysis. Alternatively, according to another preferred embodiment, the alteration of steady state level of a polypeptide is determined directly by quantifying the amount of a polypeptide. Quantifying a polypeptide amount may be carried out by any known technique such as Western blotting or immunoassay using an antibody raised against a polypeptide. The skilled person will understand that alternatively or in combination with the quantification of a nucleic acid sequence and/or the corresponding polypeptide, the quantification of a substrate of the corresponding polypeptide or of any compound known to be associated with a function or activity of the corresponding polypeptide or the quantification of said function or activity of the corresponding polypeptide using a specific assay may be used to assess the alteration of an activity or steady state level of a polypeptide.

In a method of the invention, an activity or steady-state level of a polypeptide of the invention may be altered at the level of the polypeptide itself, e.g. by providing a polypeptide of the invention to a neuronal cell from an exogenous source, or by adding an antagonist or inhibitor of a polypeptide to a neuronal cell, such as e.g. an antibody against a TF polypeptide or a dominant negative of a polypeptide or an antisense for a polypeptide. For provision of a TF polypeptide from an exogenous source, a TF polypeptide may conveniently be produced by expression of a nucleic acid encoding a polypeptide in suitable host cells as described below. An antibody against a polypeptide, an antisense or a dominant negatif of the invention may be obtained as described below. Preferably, however, an activity or steady-state level of a TF polypeptide is altered by regulating the expression level of a nucleotide sequence encoding a polypeptide.

Preferably, the expression level of a nucleotide sequence is regulated in a neuronal cell. The expression level of a polypeptide of the invention may be up-regulated (i.e. increased) by introduction of an expression construct (or vector) into a neuronal cell, whereby said expression vector comprises a nucleotide sequence encoding a TF polypeptide, and whereby a nucleotide sequence is preferably under control of a promoter capable of driving expression of a nucleotide sequence in a neuronal cell. The expression level of a TF polypeptide may also be up-regulated by introduction of an expression construct into a neuronal cell, whereby said construct comprises a nucleotide sequence encoding a factor capable of trans-activation of an endogenous nucleotide sequence encoding a TF polypeptide. Preferably, an increase or an upregulation of the expression level of a nucleotide sequence means an increase of at least 5% of the expression level of a nucleotide sequence using arrays. More preferably, an increase of the expression level of a nucleotide sequence means an increase of at least 10%, even more preferably at least 20%, at least 30%, at least 40%, at least 50%, at least 70%, at least 90%, at least 150% or more. In another preferred embodiment, an increase of the expression level of a polypeptide means an increase of at least 5% of the expression level of a polypeptide using western blotting and/or using ELISA or a suitable assay. More preferably, an increase of the expression level of a polypeptide means an increase of at least 10%, even more preferably at least 20%, at least 30%, at least 40%, at least 50%, at least 70%, at least 90%, at least 150% or more.

In another preferred embodiment, an increase of a polypeptide activity (more preferably a DNA binding and/or transcriptional activity) means an increase of at least 5% of a polypeptide activity using a suitable assay. More preferably, an increase of a polypeptide activity means an increase of at least 10%, even more preferably at least 20%, at least 30%, at least 40%, at least 50%, at least 70%, at least 90%, at least 150% or more. DNA binding activity may be assessed in an electrophoretic mobility shift assay (EMSA) using a labeled probe specific for a TF. Transcriptional activity may be assessed in an assay using a luciferase reporter construct (see the example).

Alternatively or in combination with previous embodiment, if so required for neuro(re)generation, the expression level of a polypeptide of the invention may be down regulated (i.e. decreased) by providing an antisense molecule to a neuronal cell, whereby the antisense molecule is capable of inhibiting the biosynthesis (usually the translation) of a nucleotide sequence encoding a TF polypeptide. Decreasing gene expression by providing antisense or interfering RNA molecules is described below herein and is e.g. reviewed by Famulok et al. (2002, Trends Biotechnol., 20(11): 462-466). An antisense molecule may be provided to a cell as such or it may be provided by introducing an expression construct into a neuronal cell, whereby said expression construct comprises an antisense nucleotide sequence that is capable of inhibiting the expression of a nucleotide sequence encoding a TF polypeptide, and whereby said antisense nucleotide sequence is under control of a promoter capable of driving transcription of said antisense nucleotide sequence in a neuronal cell. The expression level of a TF polypeptide may also be down-regulated by introducing an expression construct into a neuronal cell, whereby said expression construct comprises a nucleotide sequence encoding a factor capable of trans-repression of an endogenous nucleotide sequence encoding a TF polypeptide. Preferably, a nucleotide sequence capable of transrepression of an endogenous nucleotide sequence is a dominant negative of said endogenous nucleotide sequence as exemplified below.

Preferably, a decrease or a downregulation of the expression level of a nucleotide sequence means a decrease of at least 5% of the expression level of a nucleotide sequence using arrays. More preferably, a decrease of the expression level of a nucleotide sequence means an decrease of at least 10%, even more preferably at least 20%, at least 30%, at least 40%, at least 50%, at least 70%, at least 90%, at least 150% or more. In another preferred embodiment, a decrease of the expression level of a polypeptide means a decrease of at least 5% of the expression level of a polypeptide using western blotting and/or using ELISA or a suitable assay. More preferably, a decrease of the expression level of a polypeptide means a decrease of at least 10%, even more preferably at least 20%, at least 30%, at least 40%, at least 50%, at least 70%, at least 90%, at least 150% or more.

In another preferred embodiment, a decrease of a polypeptide activity (more preferably a DNA binding and/or a transcriptional activity) means a decrease of at least 5% of the polypeptide activity using a suitable assay. More preferably, a decrease of a polypeptide activity means a decrease of at least 10%, even more preferably at least 20%, at least 30%, at least 40%, at least 50%, at least 70%, at least 90%, at least 150% or more. DNA binding or transcriptional activity may be assessed as earlier defined herein.

Such an alteration (increase and/or decrease) of an activity or steady-state level of a polypeptide as earlier defined herein preferably leads to a generation or regeneration of a neuronal cell. A generation or regeneration of a neuronal cell preferably means one or more of the processes including initiation of neuronal outgrowth, neuronal outgrowth, axon elongation, target finding and reestablishment of sensory contacts, up to return of function of the deficient motory or sensory neurons. Suitable assays for generation or regeneration of a neuronal cell are provided in the Example in F11 cells and/or in DRG neurons. The assays may be used to determine if an alteration of an activity or steady state level of a polypeptide of the invention is capable of inducing neurite outgrowth and thereby capable of inducing or promoting neuronal regeneration. A method is preferably said to be for promoting generation or regeneration of a neuronal cell when the alteration of an activity or of the steady-state level of a polypeptide in a neuronal cell leads to at least one of a detectable (initiation of) neuronal outgrowth, axon elongation, target finding and reestablishment of sensory contacts and up to return of function of the deficient motory or sensory neurons all as assessed in the example. A detectable (initiation of) neuronal outgrowth and/or axon elongation preferably means a detectable increase in a median neurite total length and/or a detectable increase in the mean neurite total length. An increase in this context preferably means an increase of at least 1%, at least 2%, at least 4%, at least 5%, at least 7%, at least 10%, at least 15%, at least 20%, at least 30%, or even more of said value compared to the same value of a corresponding neuron that will not be administered a polypeptide, a nucleic acid, or a construct of the invention.

In a preferred method of the invention, regeneration of a neuronal cell is promoted by: increasing an activity or the steady-state level of a polypeptide selected from: a BHLHB3, ETS1, TRPC3, REST, PJA2, MTF1, TCEA2, PRRXL1, TCEB1, PDLIM7, ID2, TLE3, MAPK3, and a ANKRD1 and/or

decreasing an activity or the steady-state level of a polypeptide selected from: a SOX10, HES5, SREBF1, SMAD1, RTEL1, TCFE2A, CSRP3, TSC22D3, Egr1, STAT5a and a NFIL3.

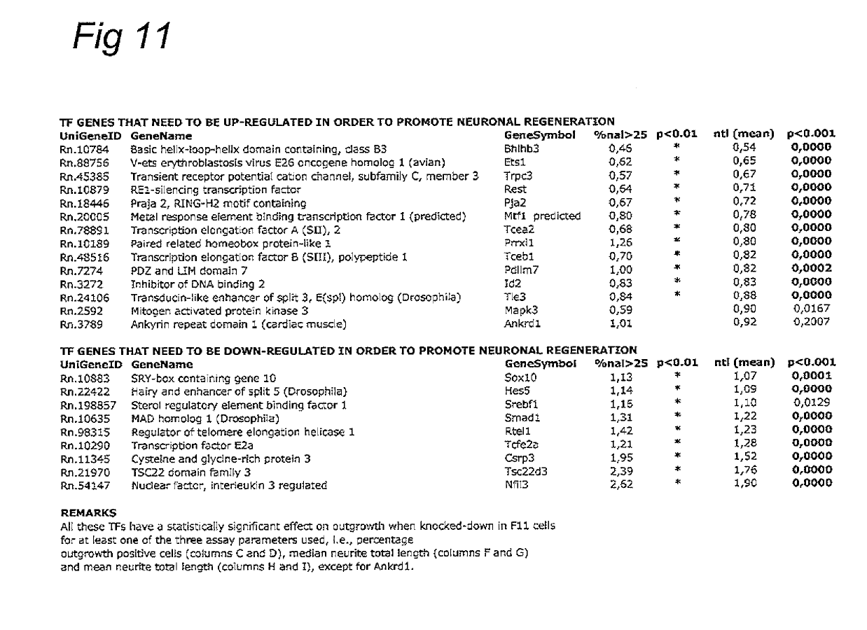

Table 1 gives an overview of the full name of each of these polypeptides, their preferred corresponding SEQ ID NOs and their accesssion number. FIG. 11 gives a further overview of all the polypeptides.

In a more preferred method, regeneration of a neuronal cell is promoted by:

increasing an activity or the steady-state level of a polypeptide selected from: a BHLJB3, TRPC3, REST, PJA2, and a TCEB1 and/or
decreasing an activity or the steady-state level of a polypeptide selected from: a RTEL1, CSRP3, TSC22D3 and a NFIL3.

In another more preferred method, regeneration of the neuronal cell is promoted by:

increasing an activity or the steady-state level of a polypeptide selected from: a BHLHB3, ETS1, TRPC3, REST, PJA2, MTF1, TCEA2, PRRXL1, TCEB1, PDLIM7, ID2, TLE3, MAPK3, and a ANKRD1 and/or
decreasing an activity or the steady-state level of a NFIL3.

In an even more preferred method, regeneration of the neuronal cell is promoted by:

increasing an activity or the steady-state level of a polypeptide selected from: a BHLJB3, TRPC3, REST, PJA2, and a TCEB1 and/or
decreasing an activity or the steady-state level of a NFIL3.

In a most preferred method, regeneration of the neuronal cell is promoted by at least decreasing an activity or the steady-state level of a NFIL3.

Optionally, an activity or the steady-state level of at least one of an ATF3, cJun, STAT3 and a CREB may be further altered. An ATF3, cJUN, STAT3 and CREB are preferably encoded by a nucleotide sequence that has at least 60, 70, 80, 85, 90, 95, 98, 99% identity with SEQ ID NO:15, 36 for ATF3, 50, 56 for cJun, 51 or 52 for STAT3 and 53 or 61 for CREB or by a nucleotide sequence that encodes an amino acid sequence that has at least 60, 70, 80, 85, 90, 95, 98, 99% identity with an amino acid sequence encoded by a nucleotide sequence selected from SEQ ID NO:15, 36 for ATF3, 50, 56 for cJun, 51 or 52 for STAT3 and 53 or 61 for CREB. An activity or steady-state level of at least one of these additional four TFs is preferably increased in order to promote generation or regeneration of a neuronal cell.

In a method of the invention, the regeneration of a neuronal cell is preferably promoted by increasing an activity or the steady-state level of a polypeptide encoded by a nucleotide sequence selected from: (a) a nucleotide sequence that has at least 80% identity with a sequence selected from SEQ ID NO.'s 2, 23, 4, 25, 6, 27, 8, 29, 9, 30, 11, 32, 12, 33, 13, 34, 16, 37, 17, 38, 18, 39, 20, 41, 43, 55, 58 and 44; and, (b) a nucleotide sequence that encodes an amino acid sequence that has at least 80% amino acid identity with an amino acid sequence encoded by a nucleotide sequence selected from SEQ ID NO.'s 2, 23, 4, 25, 6, 27, 8, 29, 9, 30, 11, 32, 12, 33, 13, 34, 16, 37, 17, 38, 18, 39, 20, 41, 43, 55, 58 and 44. A more preferred selection includes SEQ ID NO.'s 6, 27, 17, 38, 12, 33, 16, 37, 13 and 34; and the most preferred selection includes SEQ ID NO.'s. An activity or the steady-state level of a polypeptide is preferably increased by introducing a nucleic acid construct into a neuronal cell, said nucleic acid construct comprising a nucleotide sequence (encoding a polypeptide) under control of a promoter capable of driving expression of said nucleotide sequence in a neuronal cell. Suitable promoters for expression in neuronal cells are further specified herein below.

Alternatively or in combination with previous embodiment, in a method of the invention the regeneration of a neuronal cell is preferably promoted by decreasing an activity or the steady-state level of a polypeptide encoded by a nucleotide sequence selected from: (a) a nucleotide sequence that has at least 80% identity with a sequence selected from SEQ ID NO.'s 1, 22, 3, 24, 5, 26, 7, 28, 10, 31, 14, 35, 19, 40, 21, 42, 54, 59, 60, 62, 63 and 45; and, (b) a nucleotide sequence that encodes an amino acid sequence that has at least 80% amino acid identity with an amino acid sequence encoded by a nucleotide sequence selected from SEQ ID NO.'s 1, 22, 3, 24, 5, 26, 7, 28, 10, 31, 14, 35, 19, 40, 21, 42, 54, 59, 60, 62, 63 and 45. A more preferred selection includes SEQ ID NO.'s 5, 26, 3, 24, 19, 40, 21 and 42; and the most preferred selection includes SEQ ID NO.'s 21 and 42. An activity or the steady-state level of a polypeptide is preferably decreased by introducing an antisense or interfering nucleic acid molecule into a neuronal cell. An antisense or interfering nucleic acid molecule may be introduced into a cell directly “as such”, optionally in a suitable formulation, or it may be produce in situ in a cell by introducing into a cell an expression construct comprising a (antisense or interfering) nucleotide sequence that is capable of inhibiting the expression of a nucleotide sequence encoding said polypeptide, whereby, optionally, an antisense or interfering nucleotide sequence is under control of a promoter capable of driving expression of said nucleotide sequence in a neuronal cell (see herein below).

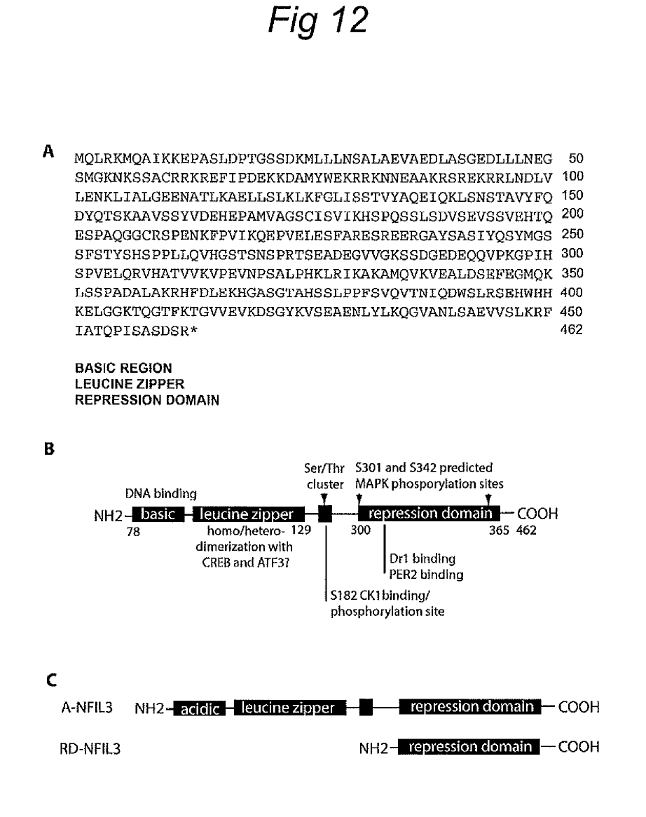

Alternatively or in combination with the antisense approach, one may also use a dominant negative approach. In this approach, a nucleic acid construct is introduced into a neuronal cell, wherein said nucleic construct comprises a dominant negative nucleotide sequence that is capable of inhibiting or downregulating an activity of a corresponding endogenous polypeptide, and wherein, optionally, a dominant negative nucleotide sequence is under the control of a promoter capable of driving expression of said dominant negative nucleotide sequence in a neuronal cell. As an example and also as a preferred embodiment, a dominant negative used is a dominant negative nucleotide encoding a dominant negative nucleotide NFIL3. More preferably, a dominant negative NFIL3 is an acidic dominant negative (A-NFIL3) or a Repression Domain NFIL3 (both as depicted in FIG. 12 and both as later more extensively disclosed).

In all embodiments exemplified, a promoter may be present in a nucleic acid construct used in the method. This promoter is preferably a neuronal specific promoter as later defined herein.

In a method of the invention, a neuronal cell preferably is a neuronal cell in need of generation or regeneration. Such cells may be found at lesions of the nervous system that have arisen from traumatic contusion, avulsion, compression, and/or transection or other physical injury, or from tissue damage either induced by, or resulting from, a surgical procedure, from vascular pharmacologic or other insults including hemorrhagic or ischemic damage, or from neurodegenerative or other neurological diseases. A neuronal cell in need of generation or regeneration may be a neuronal cell of the peripheral nervous system (PNS) but preferably is a cell of the central nervous system (CNS), in particular a neuronal cell of the corticospinal tract (CST). Although a cell in need of generation or regeneration in a method of the invention will usually be a neuronal cell, other types of cells in the environment (vicinity) of a neuronal cell may influence the ability of a neuronal cell to (re)generate). Therefore the invention expressly includes aspects relating to altering an activity or the steady-state level of a polypeptide of the invention in cells in the environment of a neuronal cell in need of (re)generation. Such environmental cells include e.g. glia cells, Schwann cells, scleptomeningeal fibroblasts, blood borne cells that invade the lesion center, astrocytes and meningeal cells.

In a further aspect, the invention pertains to a method for treating a neurotraumatic injury or a neurodegenerative disease in a subject. The method preferably comprises pharmacologically altering an activity or the steady-state level of a polypeptide of the invention as defined above in an injured or degenerated neuron in the subject. Preferably, the alteration is sufficient to induce (axonal) generation or regeneration of the injured or degenerated neuron. In this method of the invention, the neurotraumatic injury may be as described above, and likewise, the injured or degenerated neurons in the subject may be neurons of the PNS, the CNS and/or the CST.

In a method of the inventions, a neurodegenerative disease may be a disorder selected from: cerebrovascular accidents (CVA), Alzheimer's disease (AD), vascular-related dementia, Creutzfeldt-Jakob disease (CJD), bovine spongiform encephalopathy (BSE), Parkinson's disease (PD), brain trauma, multiple sclerosis (MS), amyotrophic lateral sclerosis (ALS—Lou Gehrig's disease) and Huntington's chorea.

A method of the inventions preferably comprises the step of administering to a subject a therapeutically effective amount of a pharmaceutical composition comprising a nucleic acid construct for modulating or altering an activity or steady state level of a TF polypeptide as defined herein. A nucleic acid construct may be an expression construct as further specified herein below. Preferably an expression construct is a viral gene therapy vector selected from a gene therapy vector based on an adenovirus, an adeno-associated virus (AAV), a herpes virus, a pox virus and a retrovirus. A preferred viral gene therapy vector is an AAV or Lentiviral vector. Alternatively or in combination with previous embodiment (expression construct), a nucleic acid construct may be for inhibiting expression of a TF polypeptide of the invention such as an antisense molecule or an RNA molecule capable of RNA interference (see below). Alternatively or in combination with both previous embodiments, a nucleic acid construct comprising a dominant negative of an endogenous polypeptide may be administered into a cell. In a method of the invention, a pharmaceutical composition comprising a nucleic acid construct is preferably administered at a site of neuronal injury or degeneration.

A further aspect of the invention relates to a nucleic acid construct. A nucleic acid construct comprises all or a part of a nucleotide sequence that encodes a polypeptide that comprises an amino acid sequence that is encoded by a nucleotide sequence selected from: (a) a nucleotide sequence that has at least 60, 70, 80, 85, 90, 95, 98 or 99% identity with a nucleotide sequence selected from SEQ ID NO.'s 1-45, 46, 48, 50-56, 58-63; and, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO.'s 1-45, 46, 48, 50-56, 58-63. Preferably, a nucleotide sequence is operably linked to a promoter that is capable of driving expression of the nucleotide sequence in a neuronal cell.

In a preferred nucleic acid construct, a nucleotide sequence is selected from: (a) a nucleotide sequence that has at least 60, 70, 80, 85, 90, 95, 98 or 99% identity with a sequence selected from SEQ ID NO. 2, 23, 4, 25, 6, 27, 8, 29, 9, 30, 11, 32, 12, 33, 13, 34, 16, 37, 17, 38, 18, 39, 20, 41, 43, 55, 58 and 44; and, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO.'s 2, 23, 4, 25, 6, 27, 8, 29, 9, 30, 11, 32, 12, 33, 13, 34, 16, 37, 17, 38, 18, 39, 20, 41, 43, 55, 58 and 44. A more preferred selection includes SEQ ID NO.'s; 6, 27, 17, 38, 12, 33, 16, 37, 13 and 34.

Alternatively, a nucleic acid construct of the invention comprises or consists of a nucleotide sequence that encodes an RNAi agent, i.e. an RNA molecule that is capable of RNA interference or that is part of an RNA molecule that is capable of RNA interference. Such a RNA molecule is referred to as siRNA (short interfering RNA, including e.g. a short hairpin RNA). A nucleotide sequence that encodes a RNAi agent preferably has sufficient complementarity with a cellular nucleotide sequence to be capable of inhibiting the expression of a polypeptide that comprises an amino acid sequence that is encoded by a nucleotide sequence selected from: (a) a nucleotide sequence that has at least 60, 70, 80, 85, 90, 95, 98 or 99% identity with a nucleotide sequence selected from SEQ ID NO.'s 1-45, 54-55, 59, 60, 62, 63; and, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO.'s 1-45, 54-55, 59, 60, 62, 63. In a preferred nucleic acid construct, a nucleotide sequence is selected from: (a) a nucleotide sequence that has at least 60, 70, 80, 85, 90, 95, 98 or 99% identity with a sequence selected from SEQ ID NO.'s 1, 22, 3, 24, 5, 26, 7, 28, 10, 31, 14, 35, 19, 40, 21, 42, 54 and 45; and, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO.'s 1, 22, 3, 24, 5, 26, 7, 28, 10, 31, 14, 35, 19, 40, 21, 42, 54 and 45. A more preferred selection includes SEQ ID NO.'s 5, 26, 3, 24, 19, 40, 21 and 42; and the most preferred selection includes SEQ ID NO.'s 21 and 42. Optionally, a nucleotide sequence encoding a RNAi agent is operably linked to a promoter that is capable of driving expression of a nucleotide sequence in a neuronal cell. In a preferred embodiment described earlier herein, a nucleic acid construct used in a method of the invention comprises or consists of a dominant negatif of a polypeptide of the invention as earlier defined herein. A dominant negatif is preferably designed for each of the polypeptides whose expression is to be decreased or downregulated in a method of the invention. Several strategies are already known for designing a dominant negatif of a TF. A dominant negatif is usually a truncated TF without transactivation domain but which is still able to bind DNA. Depending on the type of TF, the skilled person knows how to design such a dominant negatif TF. A dominant negatif is preferably said to have less DNA binding and/or transactivation activity on at least one target gene than its wild type counterpart. DNA binding and transactivation activities are preferably assessed as earlier defined herein. Less DNA binding and/or transactivation activity preferably means at least 5% less, at least 10% less at least 15% less, at least 20% less at least 25% less, at least 30% less at least 35% less, at least 40% less at least 45% less, at least 50% less, at least 55% less, at least 60% less at least 70% less, at least 80% less, at least 90% less, at least 95% less, or no detectable activity. A preferred polypeptide for which a dominant negatif is designed and used in a method of the invention is a dominant negatif of NFIL3. Dominant negatif of NFIL3 may be designed as described in Ahn S et al (Ahn S et al, (1998), A dominant-negative inhibitor of CREB reveals that it is a general mediator of stimulus-dependent transcription of c-fos. Mol Cell Biol 18:967-77). Two preferred distinct strategies are depicted in FIG. 12 for preparing a dominant negatif of NFIL3. In one preferred embodiment, the basic domain (DNA binding domain) present in the N terminal part of NFIL3 is substituted with an acidic domain (A-NFIL3). In this way A-NFIL3 will still be able to dimerize, will still interact with partner(s) of NFIL3, but will no longer be able to bind DNA and therefore less transactivation activity is expected. A preferred nucleic acid sequence encoding a A-NFLI3 is given as SEQ ID NO:46. A preferred A-NFIL3 is given as SEQ ID NO:47. In another preferred embodiment, a truncated NFIL3 polypeptide is prepared wherein no DNA binding domain and no leucine zipper domain are present (RD-NFIL3). In this way, a dominant negative can no longer bind DNA and can no longer dimerize. However, it can still interact with some partners via its repression domain. A preferred nucleic acid sequence of RD-NFLI3 is given as SEQ ID NO:48. A preferred RD-NFIL3 is given as SEQ ID NO:49. One may also envisage to combine the use of both types of dominant negative of NFIL3. One may also envisage to use at least one of these types of dominant negative of NFIL3 with at least one of the other TFs as defined earlier in a method as defined herein.

In a preferred embodiment, a nucleic acid construct is provided comprising a nucleotide acid sequence selected from: a) a nucleotide sequence that has at least 60, 70, 80, 85, 90, 95, 98, 99% identity with SEQ ID NO:46 or 48 or b) a nucleotide sequence that encodes an amino acid sequence that has at least 60, 70, 80, 85, 90, 95, 98, 99% identity with an amino acid sequence encoded by a nucleotide sequence selected from SEQ ID NO:46 or 48. This nucleic acid construct is preferably used in a method of the invention as earlier disclosed herein.

In a nucleic acid construct of the invention, a promoter preferably is a promoter that is specific for a neuronal cell. A promoter that is specific for a neuronal cell is a promoter with a transcription rate that is higher in a neuronal cell than in other types of cells. Preferably the promoter's transcription rate in a neuronal cell is at least 1.1, 1.5, 2.0 or 5.0 times higher than in a non-neuronal cell.

A suitable promoter for use in a nucleic acid construct of the invention and that is capable of driving expression in a neuronal cell includes a promoter of a gene that encodes an mRNA comprising a nucleotide sequence selected from: (a) a nucleotide sequence that has at least 60, 70, 80, 85, 90, 95, 98 or 99% identity with a nucleotide sequence selected from SEQ ID NO.'s 1-45, 50-56, 58-63; and, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO.'s 1-45, 50-56, 58-63. Preferably, a nucleotide sequence is selected from SEQ ID NO.'s: 7, 45, 18, 5, 26 and 28. Other suitable promoters for use in a nucleic acid construct of the invention and that is capable of driving expression in a neuronal cell include a GAP43 promoter, a FGF receptor promoter and a neuron specific enolase promoter. A promoter for use in a DNA construct of the invention is preferably of mammalian origin, more preferably of human origin.

In a preferred embodiment, a nucleic acid construct is a viral gene therapy vector selected from gene therapy vectors based on an adenovirus, an adeno-associated virus (AAV), a herpes virus, a pox virus and a retrovirus. A preferred viral gene therapy vector is an AAV or Lentiviral vector. Such vectors are further described herein below.

In a further aspect the invention relates to the use of a nucleic acid construct for modulating an activity or steady state level of a TF polypeptide as defined herein, for the manufacture of a medicament for promoting regeneration of a neuronal cell, preferably in a method of the invention as defined herein above. Preferably, a nucleic acid construct is used for the manufacture of a medicament for the treatment of a neurotraumatic injury or neurodegenerative disease, preferably in a method of the invention as defined herein above.

In yet another aspect, the invention pertains to a method for diagnosing the status of generation or regeneration of a neuron in a subject. The method comprises the steps of: (a) determining the expression level of a nucleotide sequence coding for a polypeptide of the invention in the subject's generating or regenerating neuron; and, (b) comparing the expression level of a nucleotide sequence with a reference value for expression level of a nucleotide sequence, the reference value preferably being the average value for the expression level in a neuron of healthy individuals. Preferably in a method, the expression level of a nucleotide sequence is determined indirectly by quantifying the amount of a polypeptide encoded by said nucleotide sequence. More preferably, the expression level is determined ex vivo in a sample obtained from a subject.

In yet a further aspect, the invention relates to a method for identification of a substance capable of promoting regeneration of a neuronal cell. A method preferably comprising the steps of: (a) providing a test cell population capable of expressing a nucleotide sequence encoding a TF polypeptide of the invention; (b) contacting the test cell population with a substance; (c) determining the expression level of a nucleotide sequence or an activity or steady state level of a polypeptide in a test cell population contacted with said substance; (d) comparing the expression, activity or steady state level determined in (c) with the expression, activity or steady state level of a nucleotide sequence or of a polypeptide in a test cell population that has not been contacted with a substance; and, (e) identifying a substance that produces a difference in expression level, activity or steady state level of a nucleotide sequence or a polypeptide, between a test cell population that is contacted with a substance and a test cell population that has not been contacted with said substance. Preferably, in a method the expression levels, activity or steady state levels of more than one nucleotide sequence or more than one polypeptide are compared. Preferably, in a method a test cell population comprises primary sensoric neurons (e.g. DRG neuronen), cells of the sensory neuron cell line such as e.g. the F11 cell line and/or other cells or cell lines described in the Examples herein. A test cell population preferably comprises mammalian cells, more preferably human cells. In one aspect, the invention also pertains to a substance that has been identified in said method. An increase or a decrease in expression level or activity or steady-state has preferably the same meaning as given earlier herein.

Sequence Identity

“Sequence identity” is herein defined as a relationship between two or more amino acid (polypeptide or protein) sequences or two or more nucleic acid (polynucleotide) sequences, as determined by comparing the sequences. The identity between two nucleic acid sequences is preferably defined by assessing their identity within a whole SEQ ID NO as identified herein or part thereof. Part thereof may mean at least 50% of the length of the SEQ ID NO, or at least 60%, or at least 70%, or at least 80%, or at least 90%.

In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; H is to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

Recombinant Techniques and Methods for Recombinant Production of a Polypeptide

A polypeptide for use in the present invention can be prepared using recombinant techniques, in which a nucleotide sequence encoding a polypeptide of interest is expressed in a suitable host cell. The present invention thus also concerns the use of a vector comprising a nucleic acid molecule represented by a nucleotide sequence as defined above. Preferably a vector is a replicative vector comprising on origin of replication (or autonomously replication sequence) that ensures multiplication of a vector in a suitable host for the vector. Alternatively a vector is capable of integrating into a host cell's genome, e.g. through homologous recombination or otherwise. A particularly preferred vector is an expression vector wherein a nucleotide sequence encoding a polypeptide as defined above, is operably linked to a promoter capable of directing expression of a coding sequence in a host cell for the vector.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is only active in specific types of differentiated cells/tissues, such as preferably neuronal cells or tissues.

An expression vector allows a polypeptide of the invention as defined above to be prepared using recombinant techniques in which a nucleotide sequence encoding a polypeptide of interest is expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34: 315 (describing cassette mutagenesis).

Typically, a nucleic acid encoding a polypeptide of the invention is used in an expression vector. The phrase “expression vector” generally refers to a nucleotide sequence that is capable of effecting expression of a gene in a host compatible with such sequences. These expression vectors typically include at least a suitable promoter sequence and optionally, a transcription termination signal. Additional factors necessary or helpful in effecting expression can also be used as described herein. A nucleic acid or DNA encoding a polypeptide is incorporated into a DNA construct capable of introduction into and expression in an in vitro cell culture. Specifically, a DNA construct is suitable for replication in a prokaryotic host, such as bacteria, e.g., E. coli, or can be introduced into a cultured mammalian, plant, insect, e.g., Sf9, yeast, fungi or another eukaryotic cell line.

A DNA construct prepared for introduction into a particular host typically include a replication system recognized by the host, the intended DNA segment encoding a desired polypeptide, and transcriptional and translational initiation and termination regulatory sequences operably linked to a polypeptide-encoding segment. A DNA segment is “operably linked” when it is placed into a functional relationship with another DNA segment. For example, a promoter or enhancer is operably linked to a coding sequence if it stimulates the transcription of the sequence. A DNA for a signal sequence is operably linked to a DNA encoding a polypeptide if it is expressed as a preprotein that participates in the secretion of said polypeptide. Generally, DNA sequences that are operably linked are contiguous, and, in the case of a signal sequence, both contiguous and in reading phase. However, an enhancer needs not be contiguous with a coding sequence whose transcription it controls. Linking is accomplished by ligation at convenient restriction sites or at adapters or linkers inserted in lieu thereof.

The selection of an appropriate promoter sequence generally depends upon a host cell selected for the expression of a DNA segment. Examples of suitable promoter sequences include prokaryotic, and eukaryotic promoters well known in the art (see, e.g. Sambrook and Russell, 2001, supra). A transcriptional regulatory sequence typically includes a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). An expression vector includes the replication system and transcriptional and translational regulatory sequences together with the insertion site for a polypeptide encoding segment can be employed. Examples of workable combinations of cell lines and expression vectors are described in Sambrook and Russell (2001, supra) and in Metzger et al. (1988) Nature 334: 31-36. For example, a suitable expression vector can be expressed in, yeast, e.g. S. cerevisiae, e.g., insect cells, e.g., Sf9 cells, mammalian cells, e.g., CHO cells and bacterial cells, e.g., E. coli. A host cell may thus be a prokaryotic or eukarotic host cell. A host cell may be a host cell that is suitable for culture in liquid or on solid media. A host cell is preferably used in a method for producing a polypeptide of the invention as defined above. A method comprises the step of culturing a host cell under conditions conducive to the expression of a polypeptide. Optionally a method may comprise recovery of a polypeptide. A polypeptide may e.g. be recovered from the culture medium by standard protein purification techniques, including a variety of chromatography methods known in the art per se.

Alternatively, a host cell is a cell that is part of a multicellular organism such as a transgenic plant or animal, preferably a non-human animal. A transgenic plant comprises in at least a part of its cells a vector as defined above. Methods for generating transgenic plants are e.g. described in U.S. Pat. No. 6,359,196 and in the references cited therein. Such transgenic plant may be used in a method for producing a polypeptide of the invention as defined above, said method comprising the step of recovering a part of a transgenic plant comprising in its cells the vector or a part of a descendant of such transgenic plant, whereby said plant part contains a polypeptide, and, optionally recovery of a polypeptide from said plant part. Such method is also described in U.S. Pat. No. 6,359,196 and in the references cited therein. Similarly, a transgenic animal comprises in its somatic and germ cells a vector as defined above. A transgenic animal preferably is a non-human animal. Methods for generating transgenic animals are e.g. described in WO 01/57079 and in the references cited therein. Such transgenic animal may be used in a method for producing a polypeptide of the invention as defined above, said method comprising the step of recovering a body fluid from a transgenic animal comprising a vector or a female descendant thereof, wherein the body fluid contains a polypeptide, and, optionally recovery of a polypeptide from said body fluid. Such methods are also described in WO 01/57079 and in the references cited therein. A body fluid containing a polypeptide preferably is blood or more preferably milk.

Another method for preparing a polypeptide is to employ an in vitro transcription/translation system. DNA encoding a polypeptide is cloned into an expression vector as described supra. Said expression vector is then transcribed and translated in vitro. A translation product can be used directly or first purified. A polypeptide resulting from in vitro translation typically does not contain the post-translation modifications present on a polypeptide synthesised in vivo, although due to the inherent presence of microsomes some post-translational modification may occur. Methods for synthesis of polypeptides by in vitro translation are described by, for example, Berger & Kimmel, Methods in Enzymology, Volume 152, Guide to Molecular Cloning Techniques, Academic Press, Inc., San Diego, Calif., 1987.

Gene Therapy

Some aspects of the invention concern the use of a nucleic acid construct or expression vector comprising a nucleotide sequence as defined above, wherein the vector is a vector that is suitable for gene therapy. Vectors that are suitable for gene therapy are described in Anderson 1998, Nature 392: 25-30; Walther and Stein, 2000, Drugs 60: 249-71; Kay et al., 2001, Nat. Med. 7: 33-40; Russell, 2000, J. Gen. Virol. 81: 2573-604; Amado and Chen, 1999, Science 285: 674-6; Federico, 1999, Curr. Opin. Biotechnol. 10: 448-53; Vigna and Naldini, 2000, J. Gene Med. 2: 308-16; Marin et al., 1997, Mol. Med. Today 3: 396-403; Peng and Russell, 1999, Curr. Opin. Biotechnol. 10: 454-7; Sommerfelt, 1999, J. Gen. Virol. 80: 3049-64; Reiser, 2000, Gene Ther. 7: 910-3; and references cited therein.

Particularly suitable gene therapy vectors include Adenoviral and Adeno-associated virus (AAV) vectors. These vectors infect a wide number of dividing and non-dividing cell types including neuronal cells. In addition an adenoviral vector is usually capable of high levels of transgene expression. However, because of the episomal nature of the adenoviral and AAV vectors after cell entry, these viral vectors are most suited for therapeutic applications requiring only transient expression of a transgene (Russell, 2000, J. Gen. Virol. 81: 2573-2604; Goncalves, 2005, Virol J. 2(1):43) as indicated above. A preferred adenoviral vector is modified to reduce the host response as reviewed by Russell (2000, supra). Method for neuronal gene therapy using a AAV vector is described by Wang et al., 2005, J Gene Med. March 9 (Epub ahead of print), Mandel et al., 2004, Curr Opin Mol Ther. 6(5):482-90, and Martin et al., 2004, Eye 18(11):1049-55. For neuronal gene transfer, an AAV serotype 1, 2, 5 and 8 is an effective vector and therefore a preferred AAV serotype.

A preferred retroviral vector for application in the present invention is a lentiviral based expression construct. Lentiviral vectors have the unique ability to infect non-dividing cells (Amado and Chen, 1999 Science 285: 674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10: 448-53) and Vigna et al. (2000, J Gene Med 2000; 2: 308-16).

Generally, gene therapy vectors will be as the expression vectors described above in the sense that they comprise a nucleotide sequence encoding a polypeptide of the invention to be expressed, whereby a nucleotide sequence is operably linked to the appropriate regulatory sequences as indicated above. Such regulatory sequence will at least comprise a promoter sequence. A suitable promoter for expression of a nucleotide sequence encoding a polypeptide from gene therapy vectors includes e.g. cytomegalovirus (CMV) intermediate early promoter, viral long terminal repeat promoters (LTRs), such as those from murine moloney leukaemia virus (MMLV) rous sarcoma virus, or HTLV-1, the simian virus 40 (SV 40) early promoter and the herpes simplex virus thymidine kinase promoter. Suitable neuronal promoters are described above.

Several inducible promoter systems have been described that may be induced by the administration of small organic or inorganic compounds. Such inducible promoters include those controlled by heavy metals, such as the metallothionine promoter (Brinster et al. 1982 Nature 296: 39-42; Mayo et al. 1982 Cell 29: 99-108), RU-486 (a progesterone antagonist) (Wang et al. 1994 Proc. Natl. Acad. Sci. USA 91: 8180-8184), steroids (Mader and White, 1993 Proc. Natl. Acad. Sci. USA 90: 5603-5607), tetracycline (Gossen and Bujard 1992 Proc. Natl. Acad. Sci. USA 89: 5547-5551; U.S. Pat. No. 5,464,758; Furth et al. 1994 Proc. Natl. Acad. Sci. USA 91: 9302-9306; Howe et al. 1995 J. Biol. Chem. 270: 14168-14174; Resnitzky et al. 1994 Mol. Cell. Biol. 14: 1669-1679; Shockett et al. 1995 Proc. Natl. Acad. Sci. USA 92: 6522-6526) and the tTAER system that is based on the multi-chimeric transactivator composed of a tetR polypeptide, as activation domain of VP16, and a ligand binding domain of an estrogen receptor (Yee et al., 2002, U.S. Pat. No. 6,432,705).

Suitable promoters for nucleotide sequences encoding small RNAs for knock down of specific genes by RNA interference (see below) include, in addition to the above mentioned polymerase II promoters, polymerase III promoters. The RNA polymerase III (pol III) is responsible for the synthesis of a large variety of small nuclear and cytoplasmic non-coding RNAs including 5S, U6, adenovirus VA1, Vault, telomerase RNA, and tRNAs. The promoter structures of a large number of genes encoding these RNAs have been determined and it has been found that RNA pol III promoters fall into three types of structures (for a review see Geiduschek and Tocchini-Valentini, 1988 Annu Rev. Biochem. 57: 873-914; Willis, 1993 Eur. J. Biochem. 212: 1-11; Hernandez, 2001, J. Biol. Chem. 276: 26733-36). Particularly suitable for expression of siRNAs are the type 3 of the RNA pol III promoters, whereby transcription is driven by cis-acting elements found only in the 5′-flanking region, i.e. upstream of the transcription start site. An upstream sequence element includes a traditional TATA box (Mattaj et al., 1988 Cell 55, 435-442), proximal sequence element and a distal sequence element (DSE; Gupta and Reddy, 1991 Nucleic Acids Res. 19, 2073-2075). An example of a gene under the control of the type 3 pol III promoter is a U6 small nuclear RNA (U6 snRNA), 7SK, Y, MRP, H1 and telomerase RNA genes (see e.g. Myslinski et al., 2001, Nucl. Acids Res. 21: 2502-09).

A gene therapy vector may optionally comprise a second or one or more further nucleotide sequence coding for a second or further protein. A second or further protein may be a (selectable) marker protein that allows for the identification, selection and/or screening for a cell containing the expression construct. A suitable marker protein for this purpose is e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel (2001) “Molecular Cloning: A Laboratory Manual (3^{rd} edition), Cold Spring Harbor Laboratory, Cold Spring Harbor Laboratory Press, New York.

Alternatively, a second or further nucleotide sequence may encode a protein that provides for fail-safe mechanism that allows to cure a subject from a transgenic cell, if deemed necessary. Such a nucleotide sequence, often referred to as a suicide gene, encodes a protein that is capable of converting a prodrug into a toxic substance that is capable of killing a transgenic cell in which said protein is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the IL-10 transgenic cells in the subject (see e.g. Clair et al., 1987, Antimicrob. Agents Chemother. 31: 844-849).

A gene therapy vector is preferably formulated in a pharmaceutical composition comprising a suitable pharmaceutical carrier as defined below.

RNA Interference

For knock down of expression of a specific polypeptide of the invention, a gene therapy vector or another expression construct is used for the expression of a desired nucleotide sequence that preferably encodes an RNAi agent, i.e. an RNA molecule that is capable of RNA interference or that is part of an RNA molecule that is capable of RNA interference. Such a RNA molecule is referred to as siRNA (short interfering RNA, including e.g. a short hairpin RNA). Alternatively, a siRNA molecule may directly, e.g. in a pharmaceutical composition that is administered at the site of neuronal injury or degeneration.

A desired nucleotide sequence comprises an antisense code DNA coding for the antisense RNA directed against a region of the target gene mRNA, and/or a sense code DNA coding for the sense RNA directed against the same region of the target gene mRNA. In a DNA construct of the invention, the antisense and sense code DNAs are operably linked to one or more promoters as herein defined above that are capable of expressing the antisense and sense RNAs, respectively. “siRNA” means a small interfering RNA that is a short-length double-stranded RNA that are not toxic in mammalian cells (Elbashir et al., 2001, Nature 411: 494-98; Caplen et al., 2001, Proc. Natl. Acad. Sci. USA 98: 9742-47). The length is not necessarily limited to 21 to 23 nucleotides. There is no particular limitation in the length of siRNA as long as it does not show toxicity. “siRNAs” can be, e.g. at least 15, 18 or 21 nucleotides and up to 25, 30, 35 or 49 nucleotides long. Alternatively, the double-stranded RNA portion of a final transcription product of siRNA to be expressed can be, e.g. at least 15, 18 or 21 nucleotides and up to 25, 30, 35 or 49 nucleotides long.

“Antisense RNA” is an RNA strand having a sequence complementary to a target gene mRNA, and thought to induce RNAi by binding to the target gene mRNA. “Sense RNA” has a sequence complementary to the antisense RNA, and annealed to its complementary antisense RNA to form siRNA. The term “target gene” in this context refers to a gene whose expression is to be silenced due to siRNA to be expressed by the present system, and can be arbitrarily selected. As this target gene, for example, genes whose sequences are known but whose functions remain to be elucidated, and genes whose expressions are thought to be causative of diseases are preferably selected. A target gene may be one whose genome sequence has not been fully elucidated, as long as a partial sequence of mRNA of the gene having at least 15 nucleotides or more, which is a length capable of binding to one of the strands (antisense RNA strand) of siRNA, has been determined. Therefore, genes, expressed sequence tags (ESTs) and portions of mRNA, of which some sequence (preferably at least 15 nucleotides) has been elucidated, may be selected as the “target gene” even if their full length sequences have not been determined.

The double-stranded RNA portions of siRNAs in which two RNA strands pair up are not limited to the completely paired ones, and may contain nonpairing portions due to mismatch (the corresponding nucleotides are not complementary), bulge (lacking in the corresponding complementary nucleotide on one strand), and the like. Nonpairing portions can be contained to the extent that they do not interfere with siRNA formation. The “bulge” used herein preferably comprise 1 to 2 nonpairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, in the present invention, the double-stranded RNA region of siRNAs in which two RNA strands pair up may contain both bulge and mismatched, which sum up to, preferably 1 to 7, more preferably 1 to 5 in number. Such nonpairing portions (mismatches or bulges, etc.) can suppress the below-described recombination between antisense and sense code DNAs and make the siRNA expression system as described below stable. Furthermore, although it is difficult to sequence stem loop DNA containing no nonpairing portion in the double-stranded RNA region of siRNAs in which two RNA strands pair up, the sequencing is enabled by introducing mismatches or bulges as described above. Moreover, siRNAs containing mismatches or bulges in the pairing double-stranded RNA region have the advantage of being stable in E. coli or animal cells.

The terminal structure of siRNA may be either blunt or cohesive (overhanging) as long as siRNA enables to silence the target gene expression due to its RNAi effect. The cohesive (overhanging) end structure is not limited only to the 3′ overhang, and the 5′ overhanging structure may be included as long as it is capable of inducing the RNAi effect. In addition, the number of overhanging nucleotide is not limited to the already reported 2 or 3, but can be any numbers as long as the overhang is capable of inducing the RNAi effect. For example, the overhang consists of 1 to 8, preferably 2 to 4 nucleotides. Herein, the total length of siRNA having cohesive end structure is expressed as the sum of the length of the paired double-stranded portion and that of a pair comprising overhanging single-strands at both ends. For example, in the case of 19 by double-stranded RNA portion with 4 nucleotide overhangs at both ends, the total length is expressed as 23 bp. Furthermore, since this overhanging sequence has low specificity to a target gene, it is not necessarily complementary (antisense) or identical (sense) to the target gene sequence. Furthermore, as long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end.

In addition, the terminal structure of the “siRNA” is necessarily the cut off structure at both ends as described above, and may have a stem-loop structure in which ends of one side of double-stranded RNA are connected by a linker RNA (a “shRNA”). The length of the double-stranded RNA region (stem-loop portion) can be, e.g. at least 15, 18 or 21 nucleotides and up to 25, 30, 35 or 49 nucleotides long. Alternatively, the length of the double-stranded RNA region that is a final transcription product of siRNAs to be expressed is, e.g. at least 15, 18 or 21 nucleotides and up to 25, 30, 35 or 49 nucleotides long. Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion. In the case of a stem-loop siRNA, either end (head or tail) of RNA with no loop structure may have a low molecular weight RNA. As described above, this low molecular weight RNA may be a natural RNA molecule such as tRNA, rRNA, snRNA or viral RNA, or an artificial RNA molecule.

To express antisense and sense RNAs from the antisense and sense code DNAs respectively, a DNA construct of the present invention comprises a promoter as defined above. The number and the location of the promoter in a construct can in principle be arbitrarily selected as long as it is capable of expressing antisense and sense code DNAs. As a simple example of a DNA construct of the invention, a tandem expression system can be formed, in which a promoter is located upstream of both antisense and sense code DNAs. This tandem expression system is capable of producing siRNAs having the aforementioned cut off structure on both ends. In the stem-loop siRNA expression system (stem expression system), antisense and sense code DNAs are arranged in the opposite direction, and these DNAs are connected via a linker DNA to construct a unit. A promoter is linked to one side of this unit to construct a stem-loop siRNA expression system. Herein, there is no particular limitation in the length and sequence of the linker DNA, which may have any length and sequence as long as its sequence is not the termination sequence, and its length and sequence do not hinder the stem portion pairing during the mature RNA production as described above. As an example, DNA coding for the above-mentioned tRNA and such can be used as a linker DNA.

In both cases of tandem and stem-loop expression systems, the 5′ end may be have a sequence capable of promoting the transcription from the promoter. More specifically, in the case of tandem siRNA, the efficiency of siRNA production may be improved by adding a sequence capable of promoting the transcription from the promoters at the 5′ ends of antisense and sense code DNAs. In the case of stem-loop siRNA, such a sequence can be added at the 5′ end of the above-described unit. A transcript from such a sequence may be used in a state of being attached to siRNA as long as the target gene silencing by siRNA is not hindered. If this state hinders the gene silencing, it is preferable to perform trimming of the transcript using a trimming means (for example, ribozyme as are known in the art). It will be clear to the skilled person that the antisense and sense RNAs may be expressed in the same vector or in different vectors. To avoid the addition of excess sequences downstream of the sense and antisense RNAs, it is preferred to place a terminator of transcription at the 3′ ends of the respective strands (strands coding for antisense and sense RNAs). The terminator may be a sequence of four or more consecutive adenine (A) nucleotides.

Antibodies

Some aspects of the invention concern the use of an antibody or antibody-fragment that specifically binds to a polypeptide of the invention as defined above. Methods for generating antibodies or antibody-fragments that specifically bind to a given polypeptide are described in e.g. Harlow and Lane (1988, Antibodies: A Laboratory Manual, Cold Spring Harbor Laboratory Press, Cold Spring Harbor, N.Y.) and WO 91/19818; WO 91/18989; WO 92/01047; WO 92/06204; WO 92/18619; and U.S. Pat. No. 6,420,113 and references cited therein. The term “specific binding,” as used herein, includes both low and high affinity specific binding. Specific binding can be exhibited, e.g., by a low affinity antibody or antibody-fragment having a Kd of at least about 10⁻⁴ M. Specific binding also can be exhibited by a high affinity antibody or antibody-fragment, for example, an antibody or antibody-fragment having a Kd of at least about of 10⁻⁷ M, at least about 10⁻⁸ M, at least about 10⁻⁹ M, at least about 10⁻¹⁰ M, or can have a Kd of at least about 10⁻¹¹ M or 10⁻¹² M or greater.

Peptidomimetics

A peptide-like molecule (referred to as peptidomimetics) or a non-peptide molecule that specifically binds to a polypeptide of the invention or to its receptor polypeptide and that may be applied in any of the methods of the invention as defined herein as an agonists or antagonist of a polypeptides of the invention and they may be identified using methods known in the art per se, as e.g. described in detail in U.S. Pat. No. 6,180,084 which incorporated herein by reference. Such methods include e.g. screening libraries of peptidomimetics, peptides, DNA or cDNA expression libraries, combinatorial chemistry and, particularly useful, phage display libraries. These libraries may be screened for an agonist and antagonist of a TF polypeptide by contacting the libraries with a substantially purified polypeptide of the invention, a fragment thereof or a structural analogue thereof.

Pharmaceutical Composition

The invention further relates to a pharmaceutical preparation or composition comprising as active ingredient at least one of a polypeptide, an antibody, a dominant negative, a nucleic acid or a nucleic acid construct or a gene therapy vector as defined above. The composition preferably at least comprises a pharmaceutically acceptable carrier in addition to an active ingredient.

In a preferred aspect of the invention, a nucleotide sequence or a polypeptide encoded by said nucleotide sequence or a nucleic acid construct or a dominant negatif or an antibody all as earlier defined herein are for use as a medicament. This medicament is preferably for promoting regeneration of a neuronal cell and/or for treating a neurotraumatic injury or neurodegenerative disease. All these methods have been extensively defined earlier herein.

In some methods, a polypeptide or nucleic acid or nucleic acid construct or antibody or dominant negative of the invention as purified from mammalian, insect or microbial cell cultures, from milk of transgenic mammals or other source is administered in purified form together with a pharmaceutical carrier as a pharmaceutical composition. A method of producing a pharmaceutical composition comprising a polypeptide is described in U.S. Pat. Nos. 5,789,543 and 6,207,718. The preferred form depends on the intended mode of administration and therapeutic application.

A pharmaceutical carrier can be any compatible, non-toxic substance suitable to deliver a polypeptide, an antibody, a dominant negatif or a nucleic acid or a gene therapy vector to the patient. Sterile water, alcohol, fats, waxes, and inert solids may be used as the carrier. A pharmaceutically acceptable adjuvant, buffering agent, dispersing agent, and the like, may also be incorporated into a pharmaceutical composition.

The concentration of a polypeptide or antibody or dominant negatif or nucleic acid of nucleic acid construct of the invention in a pharmaceutical composition can vary widely, i.e., from less than about 0.1% by weight, usually being at least about 1% by weight to as much as 20% by weight or more.

For oral administration, an active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. An example of an additional inactive ingredient that may be added to provide desirable colour, taste, stability, buffering capacity, dispersion or other known desirable features are red iron oxide, silica gel, sodium lauryl sulfate, titanium dioxide, edible white ink and the like. A similar diluent can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain colouring and flavouring to increase patient acceptance.

A polypeptide, antibody or dominant negatif or nucleic acid or nucleic acid construct or gene therapy vector is preferably administered parentally. A polypeptide, antibody or dominant negatif or nucleic acid or nucleic acid construct or vector for a preparation for parental administration must be sterile. Sterilisation is readily accomplished by filtration through sterile filtration membranes, prior to or following lyophilisation and reconstitution. The parental route for administration of a polypeptide, antibody or dominant negatif or nucleic acid or nucleic acid construct or vector is in accord with known methods, e.g. injection or infusion by intravenous, intraperitoneal, intramuscular, intraarterial, intralesional, intracranial, intrathecal, transdermal, nasal, buccal, rectal, or vaginal routes. A polypeptide, antibody or dominant negatif or nucleic acid or nucleic acid construct or vector is administered continuously by infusion or by bolus injection. A typical composition for intravenous infusion could be made up to contain 10 to 50 ml of sterile 0.9% NaCl or 5% glucose optionally supplemented with a 20% albumin solution and 1 to 50 μg of a polypeptide, antibody or dominant negatif or nucleic acid or nucleic acid construct or vector. A typical pharmaceutical composition for intramuscular injection would be made up to contain, for example, 1-10 ml of sterile buffered water and 1 to 100 μg of an polypeptide, antibody or dominant negatif or nucleic acid or nucleic acid construct or vector of the invention. Methods for preparing a parenterally administrable composition is well known in the art and described in more detail in various sources, including, for example, Remington's Pharmaceutical Science (15th ed., Mack Publishing, Easton, Pa., 1980) (incorporated by reference in its entirety for all purposes).

For therapeutic applications, a pharmaceutical composition is administered to a patient suffering from a neurotraumatic injury or a neurodegenerative disease in an amount sufficient to reduce the severity of symptoms and/or prevent or arrest further development of symptoms. An amount adequate to accomplish this is defined as a “therapeutically-” or “prophylactically-effective dose”. Such effective dosages will depend on the severity of the condition and on the general state of the patient's health. In general, a therapeutically- or prophylactically-effective dose preferably is a dose, which is sufficient to reverse the symptoms, i.e. to restore function of the sensory and/or motory neurons to an acceptable level, preferably (close) to the average levels found in normal unaffected healthy individuals.

In a present method of the invention, a polypeptide or antibody or dominant negatif or nucleic acid or nucleic acid construct or vector is usually administered at a dosage of about 1 μg/kg patient body weight or more per week to a patient. Often dosages are greater than 10 μg/kg per week. A dosage regime can range from 10 μg/kg per week to at least 1 mg/kg per week. Typically a dosage regime may be 10 μg/kg per week, 20 μg/kg per week, 30 μg/kg per week, 40 μg/kg week, 60 μg/kg week, 80 μg/kg per week and 120 μg/kg per week. In a preferred regime 10 μg/kg, 20 μg/kg or 40 μg/kg is administered once, twice or three times weekly. Treatment is preferably administered by parenteral route.

Microarrays

Another aspect of the invention relates to a microarray (or other high throughput screening device) comprising a nucleic acid, polypeptide or antibody as defined above. A microarray is a solid support or carrier containing one or more immobilised nucleic acid or polypeptide fragment for analysing a nucleic acid or amino acid sequence or mixtures thereof (see e.g. WO 97/27317, WO 97/22720, WO 97/43450, EP 0 799 897, EP 0 785 280, WO 97/31256, WO 97/27317, WO 98/08083 and Zhu and Snyder, 2001, Curr. Opin. Chem. Biol. 5: 40-45). Microarray comprising a nucleic acid may be applied e.g. in a method for analysing genotypes or expression patterns as indicated above. Microarrays comprising a polypeptide may be used for detection of suitable candidates of substrates, ligands or other molecules interacting with said polypeptide. Microarrays comprising an antibody may be used for in a method for analysing expression patterns of a polypeptide as indicated above.

General

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a polypeptide, a nucleic acid, a nucleic acid construct, an antibody or a dominant negatif as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “approximately” or “about” when used in association with a numerical value (approximately 10, about 10) preferably means that the value may be the given value of 10 more or less 1% of the value.

All patent and literature references cited in the present specification are hereby incorporated by reference in their entirety.

The following examples are offered for illustrative purposes only, and are not intended to limit the scope of the present invention in any way.

### EXAMPLE

Here, we applied recent advances in genomics technologies methods to uncover the gene regulatory network underlying successful neuronal regeneration. In particular, simple and robust cellular models combined with large scale application of gene expression profiling, RNA interference, TF binding site prediction and TF-promoter binding analysis, allowed an accurate reconstruction of gene regulatory networks and prediction of the key components within these networks (Blais and Dynlacht, 2005; Lee et al., 2002; Tegner and Bjorkegren, 2007). Specifically, we performed an siRNA-based screen on a large set of TFs that were previously shown to be early and differentially regulated in DRG neurons following either peripheral or central nerve crush (Stam et al., 2007), followed by analysis of the transcriptional regulatory properties of one of these TFs, nuclear factor regulated by IL-3 (NFIL3). Our data show that NFIL3 and CREB form a conserved gene regulatory network motif in which NFIL3 acts as a negative feedback regulator of CREB-induced gene expression and represses the expression of regeneration-associated genes, including Arg1 and Gap43. Intervention in transcriptional regulatory negative feedback loops might provide a powerful way to enhance neuronal regeneration-associated gene expression for therapeutic purposes.

### Results

### High-Content Screening Identifies Novel Transcriptional Regulators of DRG Neuron Outgrowth

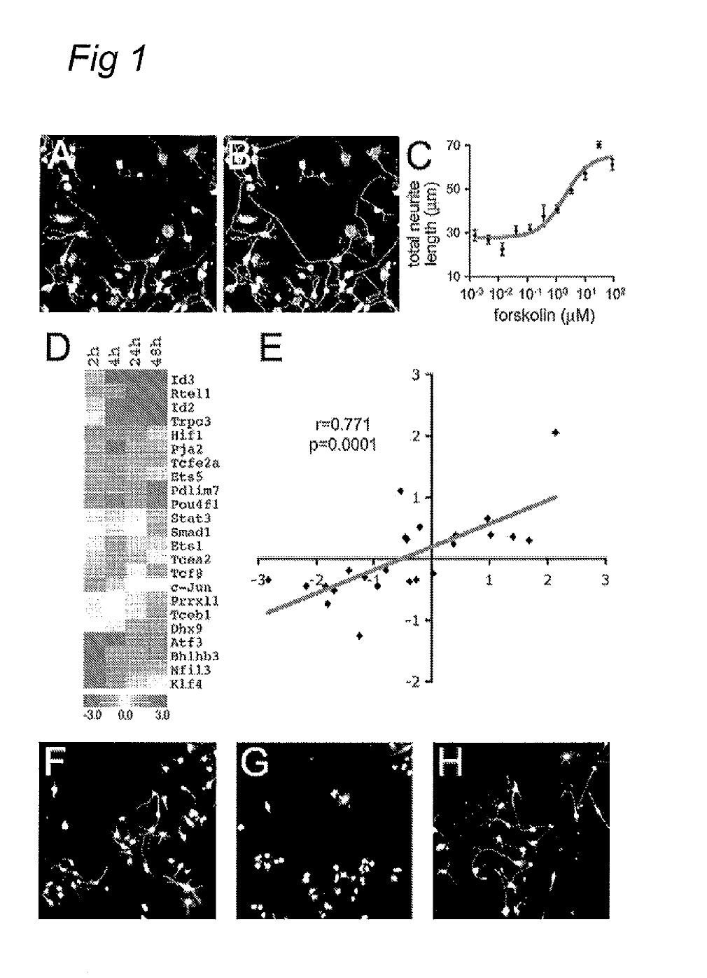

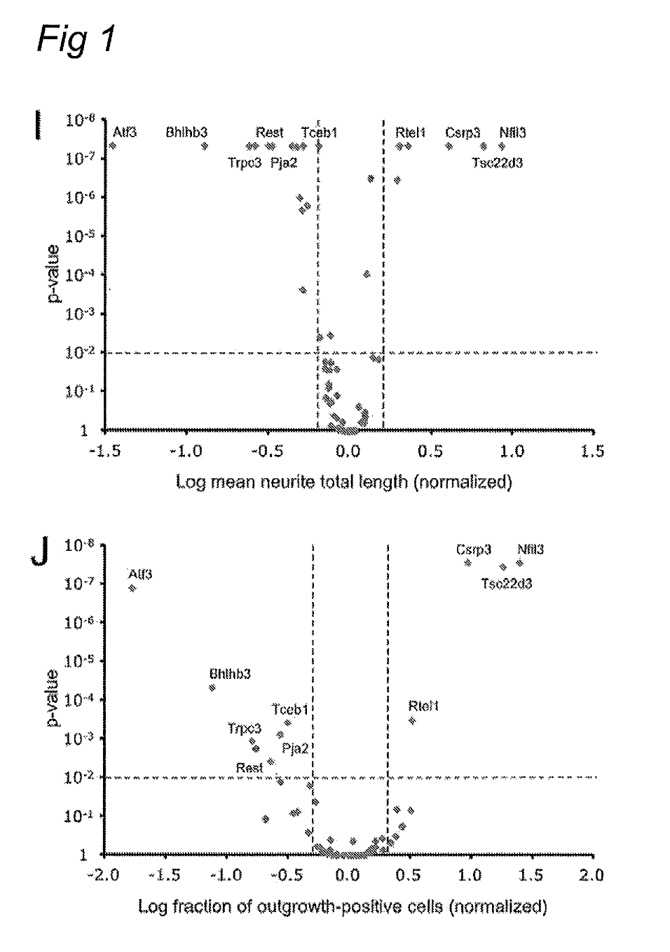

F11 cell were used to test 62 TFs for their ability to regulate neurite outgrowth. These 62 TFs include 30 TFs that were previously shown to be differentially regulated in DRG neurons following sciatic nerve injury compared with dorsal root injury (Stam et al., 2007), as well as 32 putative transcriptional regulators based on sequence similarity, subcellular localization or domain architecture (see Supplemental Figure S1). F11 cells are neuroblastoma cells derived from rat embryonic DRG neurons (Platika et al., 1985). They express many DRG neuron markers (Boland and Dingledine, 1990; Francel et al., 1987) and display cAMP-induced neurite outgrowth (Ghil et al., 2000). F11 cells are suitable for high-content screening approaches because high transfection efficiencies (>90%) can reproducibly be obtained and neurite outgrowth can be quantified in an automated and reproducible manner (FIGS. 1A-C). To validate F11 cells as a model for regenerating DRG neurons, we used quantitative real-time PCR (qPCR) to measure the expression of the 23 TFs that showed the most significant differential regulation following either sciatic nerve crush or dorsal root crush. Results indicate that these TFs show a similar up- or down-regulation in forskolin-stimulated F11 cells (FIGS. 1D-E).

To knock-down TF expression we used Dharmacon siRNA SMARTpools. Each pool consists of four individual siRNAs, allowing effective knock-down at lower siRNA concentrations and reducing concentration-dependent off-target effects (Jackson et al., 2003; Semizarov et al., 2003). Systematic knock-down of all 62 TFs followed by high-throughput automated analysis of neurite lengths showed that 19 TFs significantly affect neurite length per cell when knocked down (p<0.01 and effect size>1 standard deviation of the combined negative controls; see Experimental Procedures for details). Knock-down of 10 out of these 19 TFs also had a significant effect on the proportion of outgrowth-positive cells per well. Examples of reduced neurite outgrowth in ATF3 knock-down cells and enhanced neurite outgrowth in NFIL3 knock-down cells are shown in FIGS. 1F-H. The effects of knocking down each individual TF are shown in FIGS. 1I-J and in Supplemental Table 1. All significant effects were observed in at least two independent experiments. To validate the specificity of our siRNA knock-down approach we knocked-down the 10 candidate TFs which scored positive in both assays (neurite total length per cell and proportion of outgrowth-positive cells per well) using each of the four individual siRNAs constituting the siRNA pool (Supplemental Figure S2). In eight cases, the pool-induced effect was observed for at least two individual siRNAs. For two TFs (BHLHB3 and RTEL1) the pool-induced effect was observed for only one of the individual siRNAs.

### Sciatic Nerve Injury, but not Dorsal Root Injury Induces NFIL3 Expression in DRG Neurons

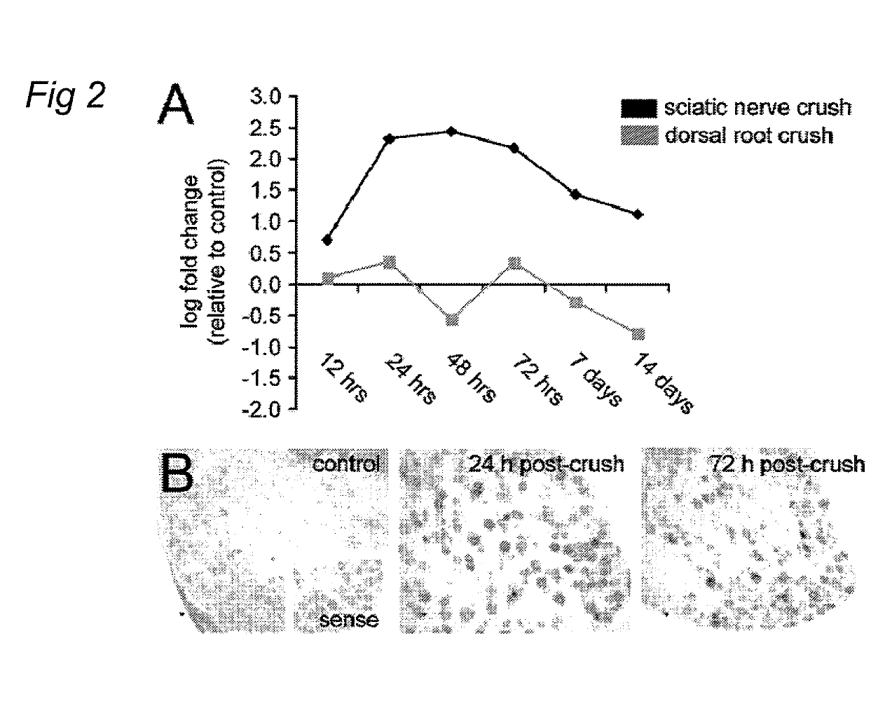

Functional screening consistently identified the bZIP transcription factor NFIL3 as the strongest repressor of neurite outgrowth. We first corroborated the temporal expression of NFIL3 mRNA after neuronal injury in vivo. qPCR analysis revealed a robust and early up-regulation of NFIL3 mRNA after sciatic nerve crush, but not after dorsal root crush (FIG. 2A), indicating that NFIL3 up-regulation is specifically correlated with successful regeneration. Between 24 h and 72 h a 5-fold up-regulation was observed compared with un-lesioned controls. At 14 days expression has dropped to a 2-fold increase relative to un-lesioned controls. To confirm up-regulation and determine the cellular source of NFIL3 mRNA we performed in situ hybridization. NFIL3 mRNA is almost absent in DRGs of control animals, but is abundantly expressed in most neurons at 24 and 72 hours after sciatic nerve crush (FIG. 2B). Together, these data suggest a role for NFIL3 in successful regeneration of DRG neurons.

cAMP Induces NFIL3 Expression in F11 Cells

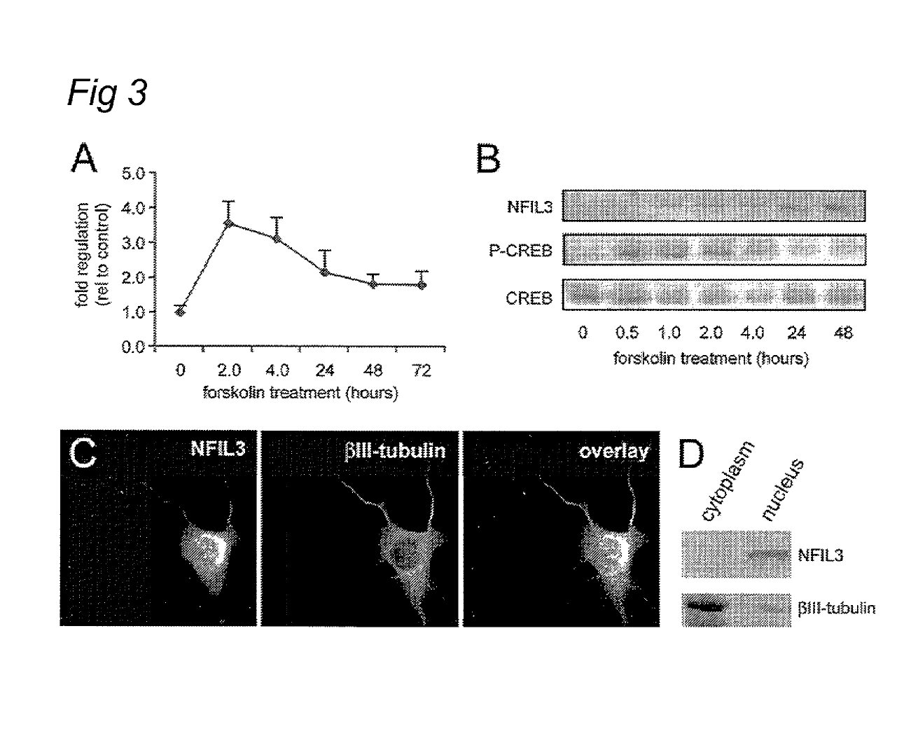

We next asked whether the lesion-induced expression of NFIL3 in DRGs could also be observed in forskolin-stimulated F11 cells. Forskolin raises intracellular cAMP levels and is a prominent neurite outgrowth stimulus for F11 cells (FIG. 1C). When F11 cells were exposed to forskolin, a rapid 3- to 4-fold induction of NFIL3 mRNA was observed within 2 hours post-stimulation which gradually stabilized at 2-fold at 48 hours post-stimulation (FIG. 3A). The pattern of forskolin-induced NFIL3 mRNA expression correlates with the initiation and elongation of neurite outgrowth and is similar to the expression pattern observed in DRGs (see FIG. 2A) but on a different time scale. In regenerating DRG neurons, increased cAMP levels are associated with activation of CREB which is required for successful regeneration (Gao et al., 2004; Qiu et al., 2002). We therefore also compared the temporal patterns of CREB activation and NFIL3 protein expression in forskolin-stimulated F11 cells. Forskolin induces a rapid activation of CREB in F11 cells. Within 30 minutes phospho-CREB levels are induced as shown by Western blotting (FIG. 3B). NFIL3 protein levels only start to increase one hour after forskolin stimulation (FIG. 3B). These data show that CREB activation precedes NFIL3 expression and suggest that NFIL3 may be downstream of CREB.

To demonstrate that NFIL3 is a nuclear protein in F11 cells we studied its localization by immunofluorescence (FIG. 3C) and by cellular fractionation followed by Western blotting (FIG. 3D). Both approaches clearly demonstrate nuclear localization of NFIL3 in line with its role as TF. No differences were observed in the ratio of cytoplasmic versus nuclear staining over the forskolin stimulation period (data not shown). Thus, NFIL3 expression in F11 cells is induced by cAMP, follows activation of CREB, and is confined to the nucleus.

Knock-Down but not Overexpression of NFIL3 Affects Neurite Outgrowth from F11 Cells

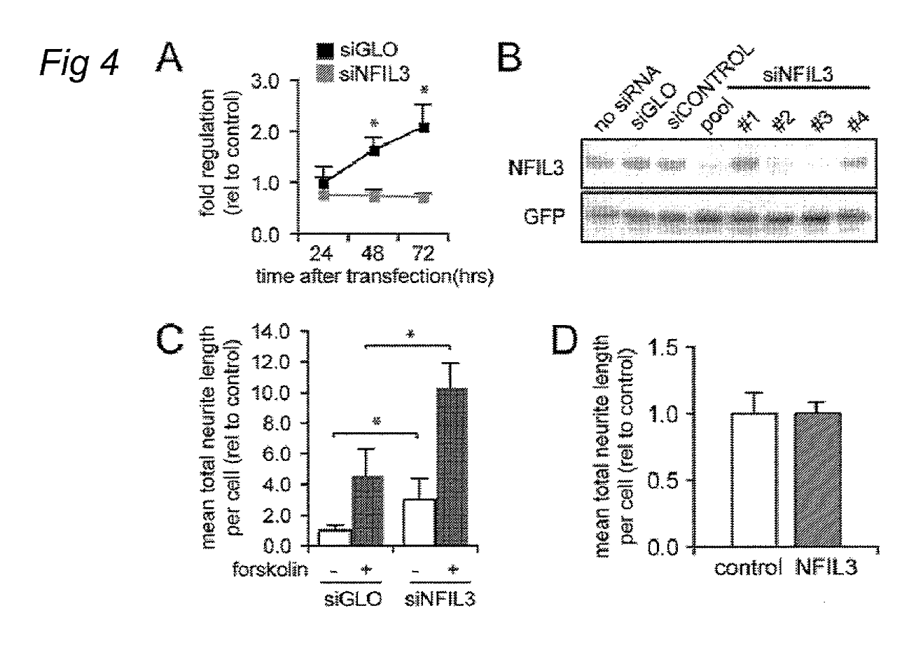

Our high-content screening results showed that NFIL3 knock-down causes enhanced neurite outgrowth from F11 cells (see FIG. 1 H-J). To demonstrate that these effects are specific, we determined NFIL3 mRNA and protein levels in F11 cells following siRNA treatment. F11 cells were transfected with siNFIL3 pool or with siGLO (control) and cultured in the presence of forskolin for 48 hours. RNA was isolated and NFIL3 mRNA levels were measured at 24 h, 48 h, and 72 h after transfection using qPCR (FIG. 4A). In siGLO-treated cells an up-regulation of NFIL3 mRNA was measured similar as before. This up-regulation was completely absent in siNFIL3-treated cells, showing that the siRNA pool effectively reduces NFIL3 mRNA levels. We next co-transfected HEK293T cells with an NFIL3 expression plasmid and either the siRNA pool or the individual siRNAs targeting NFIL3. NFIL3 protein expression was unaffected by the control siRNAs (siGLO and siCONTROL), but NFIL3 protein levels were significantly reduced in HEK293T cells co-expressing either siNFIL3 pool or individual siRNAs 2 or 3 (FIG. 4B).

To confirm our screening results, we knocked down NFIL3 both in the absence and in the presence of forskolin. Knock-down of NFIL3 resulted in a 2- to 3-fold increase in total neurite length under both conditions (FIG. 4C), confirming our screening results and showing that reduction of basal levels of NFIL3 in the absence of forskolin is sufficient to stimulate neurite outgrowth. Based on these results we expected that overexpression of NFIL3 in forskolin-stimulated cells would repress neurite outgrowth. We transfected F11 cells with either GFP (control) or NFIL3 expression constructs and cultured them for 48 hours in the presence of forskolin. Fixed and stained cells were then analyzed as above. Interestingly, we found no effect of NFIL3 overexpression on forskolin-induced neurite outgrowth compared with GFP-transfected controls (FIG. 4D). Basal neurite outgrowth levels were not affected by NFIL3 overexpression either (data not shown). From these data we conclude that forskolin stimulation results in maximally effective levels of NFIL3 and that increasing NFIL3 levels further has no additive effect. Taken together, these findings show that NFIL3 functions as a cAMP-inducible repressor of neurite outgrowth in F11 cells.

### NFIL3 is a Repressor of CREB-Mediated Gene Transcription in F11 Cells

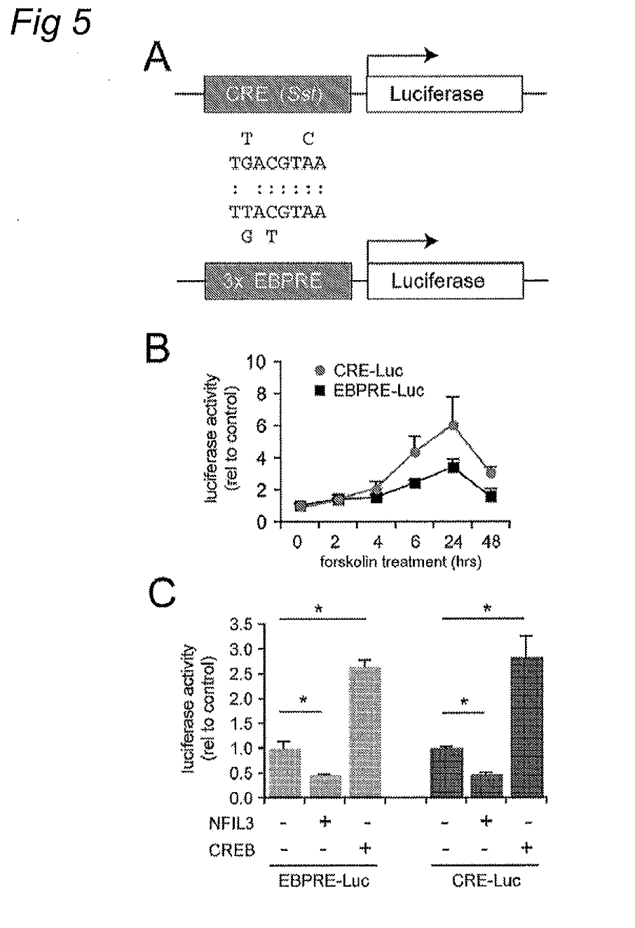

NFIL3 was previously shown to bind to the E4BP4 response element (EBPRE; TGACGT[AC]A). To study NFIL3-mediated transcription we used a luciferase reporter construct containing three repeats of the consensus EBPRE (Ozkurt and Tetradis, 2003) (FIG. 5A). Because the EBPRE consensus sequence is very similar to the cAMP response element (CRE; TGACGT[AC]A) to which CREB binds, we also used a luciferase reporter construct containing the CREB-responsive part of the rat somatostatin gene promoter (Montminy et al., 1986) to monitor CRE-mediated transcription (FIG. 5A).

Forskolin treatment of F11 cells transfected with either the EBPRE-luciferase construct or the CRE-luciferase construct resulted in an induction of luciferase activity showing peak levels at 24 h after stimulation (FIG. 5B). In order to discriminate between the effects of NFIL3 and CREB, we combined expression of the luciferase constructs with overexpression of NFIL3 or CREB. Interestingly, NFIL3 overexpression represses the activity of both the EBPRE- and the CRE-reporter, whereas both reporters are strongly induced by CREB (FIG. 5C). These results indicate that NFIL3 and CREB compete for EBPRE and CRE binding sites and have opposite effects, suggesting that NFIL3 is a repressor of CREB-mediated transcription during neurite outgrowth. Thus, the increase in reporter activity in forskolin-induced F11 cells observed at 24 h after stimulation followed by a reduction at 48 h after stimulation (FIG. 5A) matches the initial activation of CREB and the subsequent expression of NFIL3 as observed in FIG. 3B.

### NFIL3 Represses the Expression of Regeneration-Associated Genes

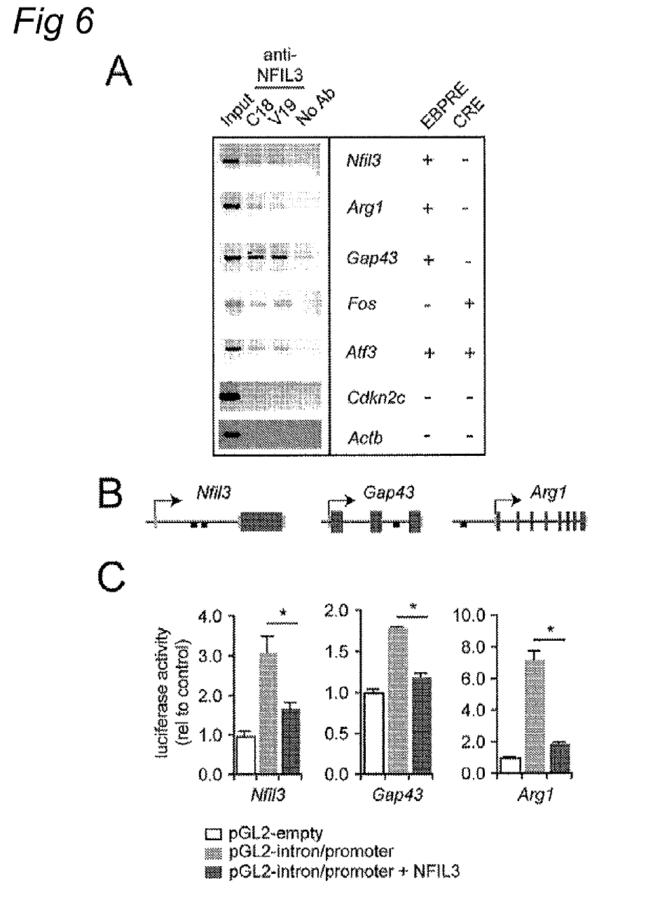

To test whether NFIL3 is also able to repress the expression of known regeneration-associated target genes, we first searched for target genes containing CRE and EBPRE sites. Of the genes that were previously shown to be regulated during regeneration (Stam et al., 2007), 67 contain predicted CRE or EBPRE sites, including Arg1, Gap43, Fos, Atf3 and Nfil3 itself (FIG. 6A). To establish direct binding of NFIL3 to these sites we performed chromatin immunoprecipitation on forskolin stimulated F11 cells transfected with an NFIL3 expression plasmid. Co-precipitated DNA was amplified using primers specific for the sequences surrounding each predicted binding site. We found that two different antibodies against NFIL3 precipitated the promoters of Arg1, Gap43, Fos, Atf3 and Nfil3, but not the promoters of beta-actin and Cdkn2c, two genes that do not contain EBPRE or CRE sites (FIG. 6A). NFIL3 thus seems to specifically associate with the promoters of predicted target genes in forskolin stimulated F11 cells, including its own promoter.

Because physical binding of a transcription factor to a gene under these conditions does not prove regulation of that gene by the transcription factor, we next cloned the predicted binding site regions of Nfil3, Gap43 and Arg1 in a luciferase reporter plasmid. These reporter plasmids were introduced in HEK293T cells either with or without an NFIL3 expression plasmid. Expression of the reporters alone resulted in an increased luciferase activity compared with empty luciferase constructs, and co-expression of NFIL3 almost completely reduced luciferase activity to basal levels (FIG. 6B). Importantly, a peripheral myelin PO promoter-luciferase construct which lacks EBPRE sites (Brown and Lemke, 1997) was not repressed by NFIL3 (data not shown). These data demonstrate that in addition to the direct binding of NFIL3 to EBPRE sites in these genes, NFIL3 also represses gene expression mediated by these sites, showing that NFIL3 is a repressor of regeneration-associated gene expression. Interestingly, the direct binding and negative regulation by NFIL3 on its own promoter indicates the presence of a negative feedback loop.

NFIL3 Represses Outgrowth from Primary Adult DRG Neurons in Culture

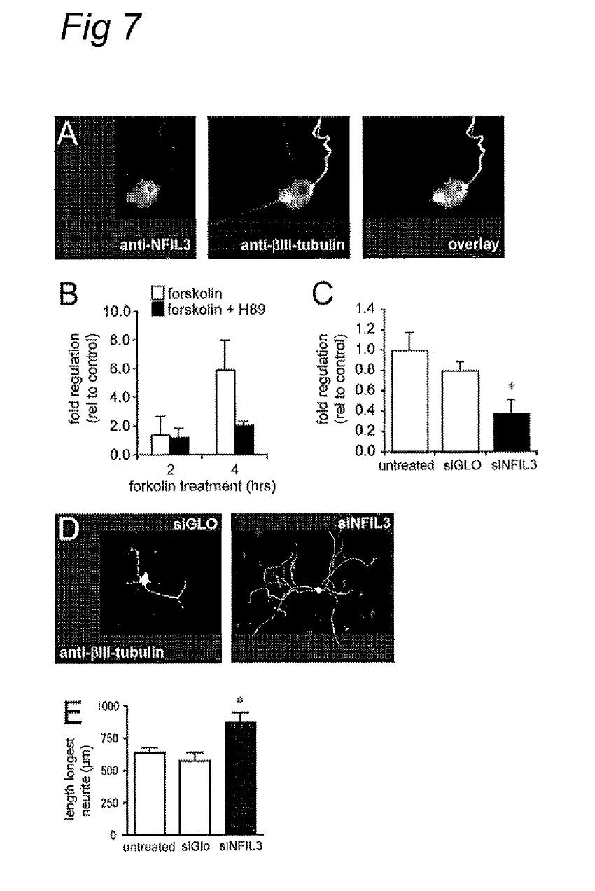

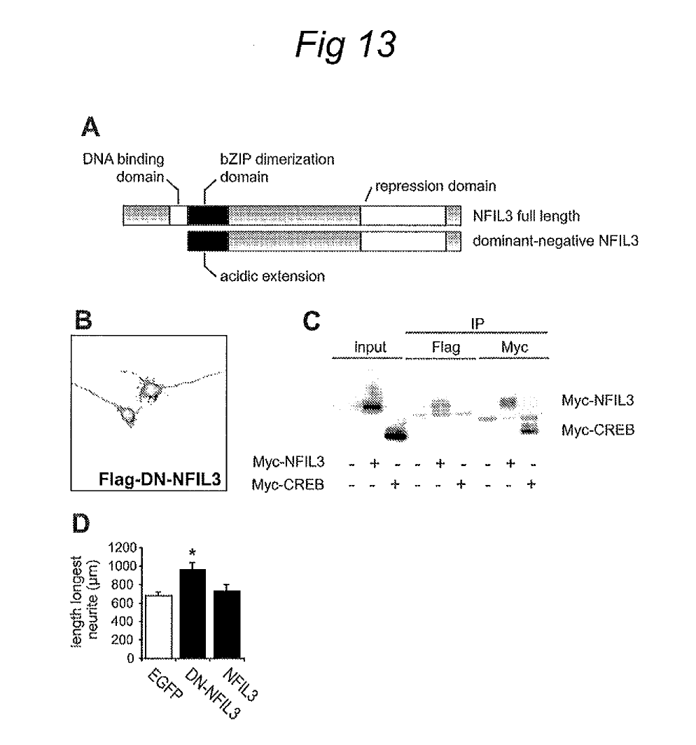

To evaluate the biological relevance of the results obtained in F11 cells we performed similar experiments in primary cultures of adult DRG neurons. Immunostaining showed that primary adult DRG neurons express NFIL3 protein, and that NFIL3 is primarily localized in the nucleus (FIG. 7A). qPCR measurements demonstrate that in forskolin-stimulated DRG neurons NFIL3 mRNA is up-regulated 5-fold at 4 h after stimulation (FIG. 7B). Up-regulation of NFIL3 mRNA is completely blocked by the PKA inhibitor H89, showing that cAMP-induced expression of NFIL3 is downstream of PKA. Next, we transfected cultured DRG neurons with the NFIL3 siRNA pool. Using the Amaxa nucleofection method we were able to reach transfection efficiencies of ˜70%, resulting in an overall reduction of NFIL3 mRNA levels of 50-60% as measured by qPCR (FIG. 6E). Transfected neurons were cultured for 48 hours and neurites were measured. We observed a 1.5-fold increase in neurite length in primary adult DRG neurons following knock-down of NFIL3 as compared with untreated or siGLO-transfected neurons (FIGS. 7D and 7E). To exclude the possibility that the siRNA-induced increase in neurite outgrowth is due to off-target effects, we also over-expressed a dominant-negative NFIL3 protein in primary adult DRG neurons. The dominant-negative NFIL3 protein lacks the basic DNA-binding domain and the nuclear localization sequence, resides in the cytoplasm, and specifically interacts with NFIL3 and not with CREB (FIG. 13). A similar dominant-negative CREB protein was previously shown to specifically inhibit CREB function and reduce DRG neuron outgrowth (Ahn et al. 1998; Gao et al. 2004). Overexpression of dominant-negative NFIL3 resulted in a similar increase in neurite outgrowth as observed in NFIL3 siRNA-transfected neurons (FIG. 13). These results confirm an important role for NFIL3 in repressing the neurite outgrowth response of injured adult DRG neurons.

### Discussion

Axonal damage in the PNS activates a regeneration-associated gene program which enables injured neurons to successfully regenerate and reinnervate target cells (Caroni, 1997; Raivich and Makwana, 2007; Skene, 1989). Coordination of the regeneration-associated gene program requires transcriptional regulation (Smith and Skene, 1997), but the underlying transcriptional regulatory mechanisms remain largely unknown. Based on in vivo gene expression data published earlier (Stam et al., 2007) we investigated the role of 62 TFs in neurite outgrowth from DRG-like F11 cells. This resulted in the identification of ten TFs that significantly affect neurite outgrowth following siRNA-mediated knock-down. Nine of these TFs have not previously been implemented in neuronal regeneration. Furthermore, our study shows that NFIL3 is a repressor of regeneration-associated genes, and is together with CREB involved in a novel transcriptional regulatory mechanism for neuronal regeneration-associated gene expression.

Our screen resulted in a relatively low number of positive hits (10 out of 62), given that all 62 TFs were initially found to be specifically regulated during successful regeneration. 23 out of 62 TF were found to have a statistical significant effect in at least one of both assays described herein. Table 1 lists the 23 TF and ATF3. 10 out of 62 TF were found to have a statistical significant effect in both assays described herein. These 10 TF are listed in claim 4. This might in part be due to the stringent selection criteria that were applied in order to eliminate false-positives. In addition however there may be several biological reasons why knock-down of many TFs did not produce an effect in our screen. First, our screening approach was focussed on neurite outgrowth, but for successful regeneration to occur, other cellular activities may be required which were not measured. Second, in order to facilitate high-throughput screening we used F11 cells and not primary DRG neurons, and there may be cell type specificity issues that affected the screening results. Third, there may be redundancy in TF function, and knocking down individual TFs may not always have resulted in an effect on neurite outgrowth because of compensation by other TFs. Finally, TFs may act synergistically, and knocking down individual TFs may have led to partial loss of function resulting in non-significant effects.

Surprisingly, we do not always observe a clear correlation between the in vivo gene regulation of a TF and the siRNA-induced effect on neurite outgrowth. Knock-down of ATF3, BHLHB3 and TCEB1 (up-regulated after sciatic nerve crush), and of PJA2, TRPC3 and REST (down-regulated after sciatic nerve crush) resulted in inhibition of neurite outgrowth. On the other hand, knock-down of NFIL3 and CSRP3 (up-regulated after sciatic nerve crush) and of RTEL and TSC22D3 (down-regulated after sciatic nerve crush) resulted in enhanced neurite outgrowth. Little if anything is known about the possible roles of these TFs in neuronal outgrowth, but it is at least intriguing that injured neurons up-regulate TFs that have opposite roles in the process of neurite outgrowth. This suggests that some injury-induced TFs may be required for regeneration-associated processes that are not directly related to neuronal outgrowth. Alternatively, outgrowth-promoting and outgrowth-inhibiting TFs may be required together in order to keep growth velocities within a physiologically optimal range. Finally, TFs may be regulated as part of evolutionary fixed gene regulatory motifs in which the expression of TFs is coupled irrespective of their functional context.

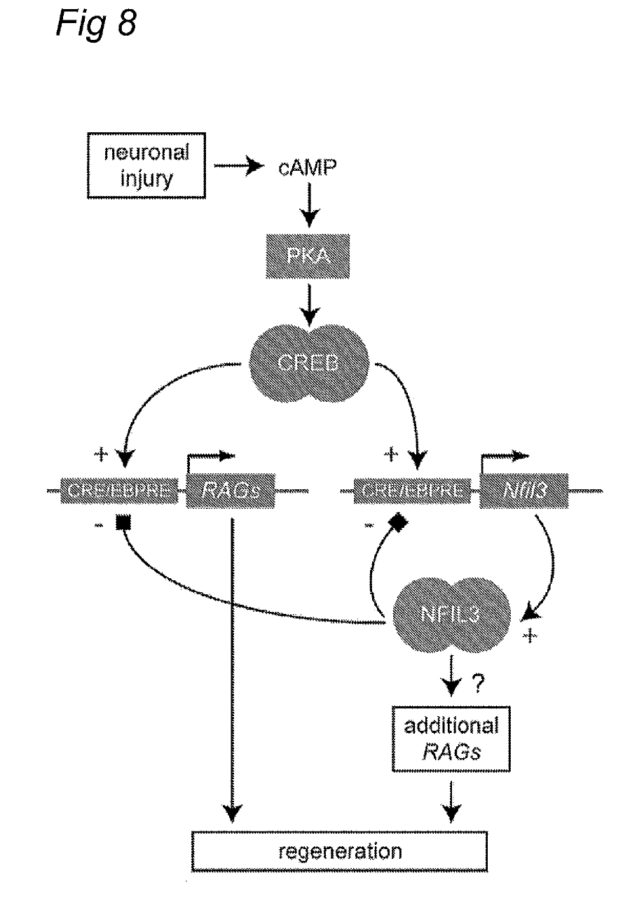

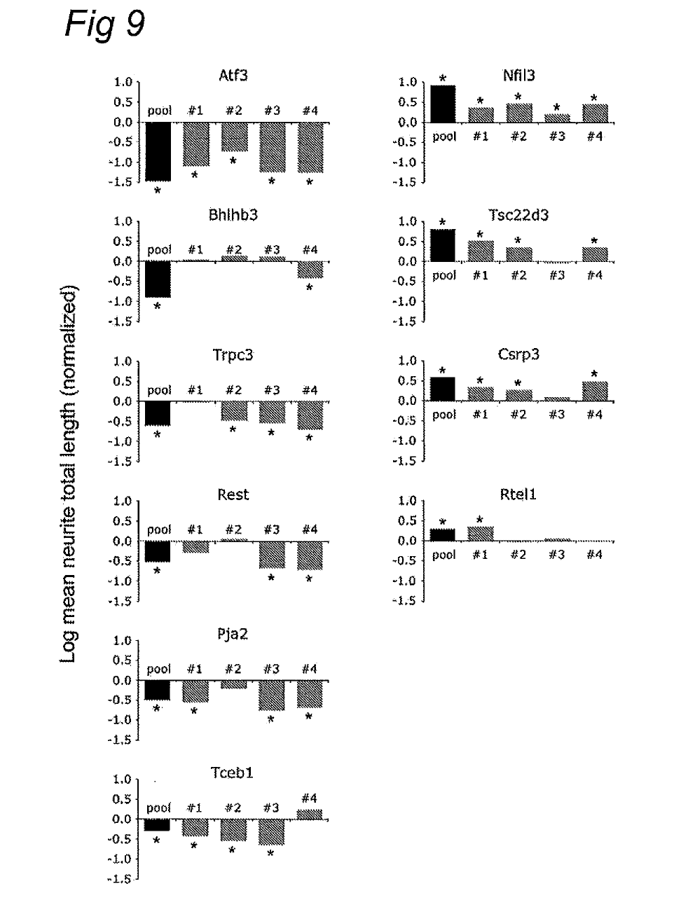

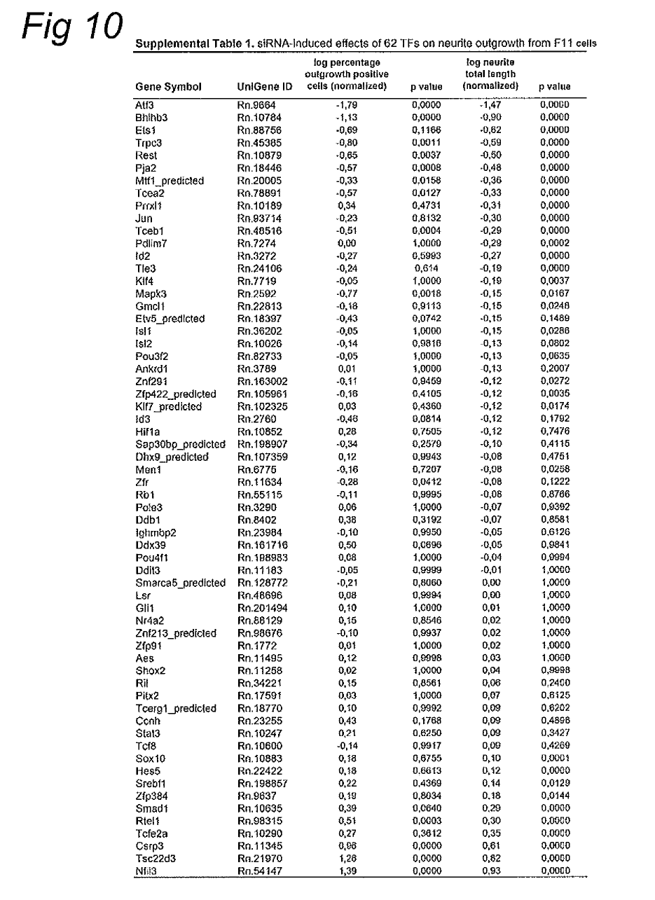

Our data indicate that NFIL3 might be part of an evolutionary conserved gene regulatory motif. NFIL3 is induced in injured DRG neurons by cAMP and PKA (FIG. 7B). Thus, the same signalling pathway that causes activation of CREB, which is essential for the induction of regenerative neurite outgrowth (Gao et al., 2004; Qiu et al., 2002), also induces NFIL3 as a repressor of outgrowth. Our data suggest that NFIL3 expression is induced by CREB; NFIL3 expression follows phospho-CREB induction in forskolin-stimulated F11 cells (FIG. 3B) and the Nfil3 gene contains two functional EBPRE sites, which according to our data can also be bound by CREB (FIG. 5C). Nevertheless NFIL3 expression in regenerating cultured DRG neurons is a consequence of the activation of CREB by cAMP and PKA, and an important finding in our study is that NFIL3 competes with CREB for EBPRE and CRE binding sites in gene regulatory regions of regeneration-associated genes. These binding sites appear to be functionally equivalent: CREB enhances gene expression via both sites, whereas NFIL3 represses gene expression via both sites. These results are consistent with a model in which, as part of the cAMP regulated gene program, NFIL3 is up-regulated to control the CREB-mediated transcriptional response, acting as a negative feedback regulator (FIG. 8). The fact that NFIL3 expression follows CREB activation might suggest that CREB initially triggers a vigorous outgrowth response, and that NFIL3 serves to attenuate growth later on, perhaps to allow regeneration to proceed at a physiologically optimal speed. Also, we cannot exclude that NFIL3 regulates the expression of other regeneration-associated genes independent of CREB.

The role for NFIL3 as a negative feedback repressor in neuronal outgrowth is similar to its function in modulating the circadian clock. In the central circadian clock residing in the suprachiasmatic nucleus, but also in peripheral clock mechanisms, NFIL3 expression cycles with the diurnal rhythm opposite to the transcriptional activators DBP, TEF and HLF (Doi et al., 2001; Mitsui et al., 2001). Here, it also competes with activators for promoter elements in clock-controlled genes and represses transcription, resulting in an oscillating expression pattern. In other systems NFIL3 is involved in the regulation of apoptosis; the C. Elegans ortholog CES-2 (cell death selector-2) acts as a pro-apoptotic factor, whereas in mammalian lymphoid tissues NFIL3 has strong anti-apoptotic effects (Ikushima et al., 1997; Kuribara et al., 1999; Metzstein et al., 1996). Here, we focused on the regulation of neurite outgrowth but we cannot exclude the possibility that NFIL3 also affects neuronal survival following nerve injury. In contrast to our findings, work by Junghans et al. (2004) showed that in developing chick spinal cord motor neurons NFIL3 has a positive effect on neuronal survival and axonal outgrowth. This might be explained by differences in cellular context and/or developmental state. For example, in motor neuron development, NFIL3 was shown to act downstream of PI3K, whereas in our studies cAMP levels were raised to induce neurite outgrowth. Different signalling pathways may result in the expression of different transcriptional (co-)activators, which in turn may determine the observed differences in NFIL3 effects on neuronal outgrowth.

In conclusion, our work demonstrates that CREB and NFIL3 form a regeneration-associated gene regulatory network motif in which CREB induces gene expression and NFIL3 provides negative feedback.

### Experimental Procedures

### Cell Culture and Transfection

F11 and HEK293T cells were maintained in Dulbeco's modified Eagle's medium (DMEM; Invitrogen, San Diego, Calif.) supplemented with 10% fetal calf serum (FCS), 100 U/ml penicillin and 100 U/ml streptomycin at 37° C. and 5% CO₂. F11 cells were transfected with Dharmacon siGENOME siRNA SMARTpools using the DharmaFECT 3 transfection reagent according to the manufacturer's instructions (Dharmacon, Lafayette, Colo.). For transfection with DNA plasmids Lipofectamine 2000 Reagent (Invitrogen) was used. HEK293T cells were co-transfected with NFIL3 expression plasmid and siRNA SMARTpools using the Dharmafect Duo transfection reagent (Dharmacon).

### High-Content Screening

F11 cells were cultured in 96 well plates (2,000 cells per well) and transfected with Dharmacon siGENOME siRNA SMARTpools. Per plate 12 siRNAs were tested (5 wells for each siRNA; outer wells were not used), including three negative controls (siCONTROL non-targeting pool; siGLO RISC-free siRNA; transfection without siRNAs) as well as one positive control (siATF3). Four hours after transfection outgrowth was induced by replacing the medium with DMEM containing 0.5% FCS and 10 μM forskolin. After 2 days cells were fixed and stained with either anti-neurofilament (N4142; Sigma-Aldrich, Basel, Switzerland) or anti-βIII-tubulin (Sigma-Aldrich) and with Hoechst 33258 (Molecular Probes, Eugene, Oreg.) (see below). Neurite outgrowth was quantified using a Cellomics KineticScan HCS Reader and the Neuronal Profiling Bioapplication (Cellomics Inc., Pittsburgh, Pa., USA). Per well 500-1,000 cells were analyzed and neurite total length per cell (cell-based analysis) and the percentage of cells per well having a neurite average length of >25 μm (population-based analysis) were calculated.

Statistics and Target Selection

Statistical significance was determined per plate by One-Way ANOVA and Kruskal Wallis test for cell-based features and by One-Way ANOVA only for well-based features. A Dunnett's post-hoc test was used for ANOVA analyses. Post-hoc multiple comparison's tests for Kruskal Wallis analyses were performed as described by Siegel and Castellan (1988). siRNA effects were compared to one of the controls (usually siGLO) and deemed significant when p<0.01. In addition to the statistical significance criterion, hits were only selected when the size effect of the siRNA was larger than 1 standard deviation of all combined negative controls throughout the screen. All positive hit effects were replicated 2-3 times using the siRNA pools, and a selection of 10 positive hits was replicated using the four individual siRNAs that comprise each siRNA pool.

### Surgical Procedures

Adult male Wistar rats were subjected to either sciatic nerve or dorsal root crush and were sacrificed at various post-injury time-points as described earlier (Stam et al., 2007). L5 and L6 DRGs were dissected and stored at −80° C. until use.

### Expression Constructs

Full-length rat NFIL3 cDNA was PCR amplified from rat whole-brain cDNA and inserted into the pcDNA3.1 expression vector (Invitrogen). The pCMV-MYC-CREB plasmid was kindly provided by Dr. A. Riccio (Johns Hopkins University School of Medicine, Baltimore, Md.) (Riccio et al., 2006). For generation of the NFIL3 dominant-negative inhibitor the C-terminal portion of NFIL3 encoding the leucine zipper was inserted into pcDNA3.1, which was modified to contain an N-terminal Flag epitope followed by a Φ10 sequence and an acidic extension as described by Ahn et al (1998).

### RNA Isolation and Quantitative Real-Time PCR

Total RNA was isolated using Trizol (Invitrogen). cDNA was synthesized from total RNA using MMLV reverse transcriptase (Invitrogen). Quantitative RT-PCR was performed on the ABI 7900HT detection system (Applied Biosystems, Foster City, Calif.) with the 2×SYBR green ready reaction mix (Applied Biosystems). GAPDH and NSE transcripts were measured for normalization.

### Western Blot Analysis

Cells were lysed in 1× Laemmli sample buffer (2% SDS, 0.2 mg/ml bromophenol blue, 0.1 M DTT, 10% glycerol in 50 mM Tris-HCl). Samples were electrophoresed on a 10% SDS-PAGE gel. Proteins were blotted onto PVDF membrane (Biorad, Hercules, Calif.), blocked with 5% low-fat milk, 1% Tween-20 in PBS. Membranes were incubated with phospho-CREB (Ser133), anti-CREB (both from Cell Signaling Technology, Danvers, Mass.) or anti-NFIL3 antibody (V19, Santa Cruz Biotechnology, Santa Cruz, Calif.), washed three times with PBS-T (PBS with 1% Tween-20) and incubated with alkaline phosphatase-conjugated secondary antibodies (1:5,000; DAKO, Glostrup, Denmark). Immunoreactivity was analyzed using the ECF detection system (Amersham Biosciences, Piscataway, N.J.).

### Luciferase Assays

The pTK-EBPRE vector (Ozkurt and Tetradis, 2003) was a kind gift of Dr. S. Tetradis (UCLA School of Dentistry, Los Angeles, Calif.). The Sst-luciferase vector (Montminy et al., 1986) was kindly provided by Dr. M. R. Montminy (Salk Institute for Biological Studies, La Jolla, Calif.). The peripheral myelin PO promoter-luciferase construct (Brown and Lemke, 1997) was a kind gift of Dr. G. Lemke (Salk Institute for Biological Studies, La Jolla, Calif.). Nfil3-, Gap43- and Arg1-luciferase constructs were created by inserting a ˜1 kb fragment encompassing the predicted EBPREs into the pGL2-BASIC-luciferase plasmid (Invitrogen). F11 or HEK293 cells were transfected with indicated constructs and medium was replaced with DMEM containing 0.5% FCS and antibiotics with or without 10 μM forskolin the next day. After 2 days, cells were lysed with Steady-Glo luciferase lysis buffer (Promega, Madison, Wis.) and luciferase activity was analyzed with a luminometer (Wallac Victor 1420; Perkin Elmer, Waltham, Mass.). The luminescent signal was corrected for transfection efficiency using LacZ measurement. Experiments were carried out in triplicate.

### Chromatin Immunoprecipitation (ChIP) Analysis

F11 cells (10⁷) were grown in 15 cm culture plates and transiently transfected with an NFIL3 expression plasmid. Chromatin complexes were cross-linked with 1% formaldehyde for 10 min. Cross-linking was stopped by addition of 125 mM Glycine for 5 min. Cells were washed with cold PBS, nuclei were extracted with cell lysis buffer (10 mM EDTA, 10 mM HEPES, 0.25% Triton X-100 supplemented with protease inhibitor cocktail), washed once in HEPES buffer (1 mM EDTA, 10 mM HEPES, 200 mM NaCl supplemented with protease inhibitor cocktail) and lysed with SDS lysis buffer (1% SDS, 10 mM EDTA in 20 mM Tris-HCl supplemented with protease inhibitor cocktail). Cross-linked chromatin was sheared by sonication (4 pulses of 15 sec on ice with 30 sec intervals). This consistently yielded DNA of 200-1,000 by in length. Cell lysates were diluted 10 times with dilution buffer (1% Triton X-100, 2 mM EDTA, 150 mM NaCl in 20 mM Tris-HCl). Immunoprecipitation was performed with goat anti-NFIL3 (C18 and V19, Santa Cruz Biotechnology) overnight with gentle rotation at 4° C. Immunoprecipitated complexes were captured with protein A/G beads (Santa Cruz Biotechnology) and pre-incubated with sonicated salmon sperm DNA by rotation at 4° C. for 2 hours. Complexes were washed subsequently with low-salt buffer (0.1% SDS, 1% Trition X-100, 2 mM EDTA, 150 mM NaCl in 20 mM Tris-HCl), high-salt buffer (0.1% SDS, 1% Triton X-100, 2 mM EDTA, 500 mM NaCl in 20 mM Tris HCl), LiCl buffer (1 mM EDTA, 250 mM LiCl, 1% deoxycholate, 1% NP-40 in 20 mM Tris-HCl) and three times with TE buffer (10 mM Tris-HCl, 1 mM EDTA). The immunoprecipitated chromatin complexes were eluted three times with 150 μl elution buffer (1% SDS, 100 mM NaHCO₃), each with shaking for 10 minutes at room temperature. The eluates were combined and proteinase K was added (215 μg/ml) and incubated at 65° C. for overnight to reverse cross-link protein-DNA complexes. DNA was purified by phenol/chloroform extraction and subsequent ethanol precipitation. Immunoprecipitated and input fractions were analyzed by PCR using gene-specific primers.

### Primary Adult DRG Neuron Culture

Adult male Wistar rats were anesthetized and decapitated. DRGs were dissected and transferred to DMEM/F12. DRGs were trimmed, desheated and enzymatically digested with collagenase type I in Hanks balanced salt solution (HBSS) and subsequently with collagenase type I and trypsin in HBS. Digestion was stopped by addition of DMEM containing 10% FCS. DRGs were mechanically dissociated with a fire-polished Pasteur pipette. Dissociated DRG neurons were transfected with the Nucleofector 96-well system (Amaxa Biosystems, Cologne, Germany) according to the manufacturer's protocol. Neurons were then plated in 24-well plates on poly-L-lysine coated coverslips in Neurobasal medium containing 2% B27 supplement (Invitrogen), 2 mM glutamine and 50 μM gentamycin, and cultured for 48 hours. Neurons were fixed and immunostained. The longest neurites of 100-200 neurons were measured.

### Immunostaining

Cells were fixed in 4% paraformaldehyde for 30 min. Cells were then washed three times with PBS and blocked with 5% goat serum, 0.5% Triton in PBS, pH 7.4 for one hour. Fixed cells were incubated with primary antibodies rabbit anti-NFIL3 (H-300, Santa Cruz Biotechnology; 1:50) and mouse anti-βIII-tubulin (Sigma; 1:500) for 2 hours, followed by three wash steps with PBS. Secondary goat anti-rabbit-Cy3 and goat anti-mouse-Cy5 were added for two hours. Coverslips were washed three times with water and mounted.
Ahn, S., Olive, M., Aggarwal, S., Krylov, D., Ginty, D. D., and Vinson, C. 1998. A dominant-negative inhibitor of CREB reveals that it is a general mediator of stimulus-dependent transcription of c-fos. Mol Cell Biol 18, 967-977.
Aigner, L., Arber, S., Kapfhammer, J. P., Laux, T., Schneider, C., Botteri, F., Brenner, H. R., and Caroni, P. (1995). Overexpression of the neural growth-associated protein GAP-43 induces nerve sprouting in the adult nervous system of transgenic mice. Cell 83, 269-278.
Alon, U. (2007). Network motifs: theory and experimental approaches. Nat Rev Genet 8, 450-461.
Blais, A., and Dynlacht, B. D. (2005). Constructing transcriptional regulatory networks. Genes Dev 19, 1499-1511.
Boland, L. M., and Dingledine, R. (1990). Expression of sensory neuron antigens by a dorsal root ganglion cell line, F-11. Brain Res Dev Brain Res 51, 259-266.
Bomze, H. M., Bulsara, K. R., Iskandar, B. J., Caroni, P., and Skene, J. H. (2001). Spinal axon regeneration evoked by replacing two growth cone proteins in adult neurons. Nat Neurosci 4, 38-43.
Broude, E., McAtee, M., Kelley, M. S., and Bregman, B. S. (1997). c-Jun expression in adult rat dorsal root ganglion neurons: differential response after central or peripheral axotomy. Exp Neurol 148, 367-377.
Brown, A. M., and Lemke, G. (1997). Multiple regulatory elements control transcription of the peripheral myelin protein zero gene. J Biol Chem 272, 28939-28947.
Cai, D., Deng, K., Mellado, W., Lee, J., Ratan, R. R., and Filbin, M. T. (2002). Arginase I and polyamines act downstream from cyclic AMP in overcoming inhibition of axonal growth MAG and myelin in vitro. Neuron 35, 711-719.
Caroni, P. (1997). Intrinsic neuronal determinants that promote axonal sprouting and elongation. Bioessays 19, 767-775.
Chong, M. S., Reynolds, M. L., Irwin, N., Coggeshall, R. E., Emson, P. C., Benowitz, L. I., and Woolf, C. J. (1994). GAP-43 expression in primary sensory neurons following central axotomy. J Neurosci 14, 4375-4384.
Chong, M. S., Woolf, C. J., Haque, N. S., and Anderson, P. N. (1999). Axonal regeneration from injured dorsal roots into the spinal cord of adult rats. J Comp Neurol 410, 42-54.
Doi, M., Nakajima, Y., Okano, T., and Fukada, Y. (2001). Light-induced phase-delay of the chicken pineal circadian clock is associated with the induction of cE4 bp4, a potential transcriptional repressor of cPer2 gene. Proc Natl Acad Sci USA 98, 8089-8094.
Francel, P. C., Harris, K., Smith, M., Fishman, M. C., Dawson, G., and Miller, R. J. (1987). Neurochemical characteristics of a novel dorsal root ganglion X neuroblastoma hybrid cell line, F-11. J Neurochem 48, 1624-1631.
Frey, D., Laux, T., Xu, L., Schneider, C., and Caroni, P. (2000). Shared and unique roles of CAP23 and GAP43 in actin regulation, neurite outgrowth, and anatomical plasticity. J Cell Biol 149, 1443-1454.
Gao, Y., Deng, K., Hou, J., Bryson, J. B., Barco, A., Nikulina, E., Spencer, T., Mellado, W., Kandel, E. R., and Filbin, M. T. (2004). Activated CREB is sufficient to overcome inhibitors in myelin and promote spinal axon regeneration in vivo. Neuron 44, 609-621.
Ghil, S. H., Kim, B. J., Lee, Y. D., and Suh-Kim, H. (2000). Neurite outgrowth induced by cyclic AMP can be modulated by the alpha subunit of Go. J Neurochem 74, 151-158.
Goutsias, J., and Lee, N. H. (2007). Computational and experimental approaches for modeling gene regulatory networks. Curr Pharm Des 13, 1415-1436.
Hanz, S., Perlson, E., Willis, D., Zheng, J. Q., Massarwa, R., Huerta, J. J., Koltzenburg, M., Kohler, M., van-Minnen, J., Twiss, J. L., and Fainzilber, M. (2003). Axoplasmic importins enable retrograde injury signaling in lesioned nerve. Neuron 40, 1095-1104.
Ikushima, S., Inukai, T., Inaba, T., Nimer, S. D., Cleveland, J. L., and Look, A. T. (1997). Pivotal role for the NFIL3/E4BP4 transcription factor in interleukin 3-mediated survival of pro-B lymphocytes. Proc Natl Acad Sci USA 94, 2609-2614.
Impey, S., McCorkle, S. R., Cha-Molstad, H., Dwyer, J. M., Yochum, G. S., Boss, J. M., McWeeney, S., Dunn, J. J., Mandel, G., and Goodman, R. H. (2004). Defining the CREB regulon: a genome-wide analysis of transcription factor regulatory regions. Cell 119, 1041-1054.
Jackson, A. L., Bartz, S. R., Schelter, J., Kobayashi, S. V., Burchard, J., Mao, M., Li, B., Cavet, G., and Linsley, P. S. (2003). Expression profiling reveals off-target gene regulation by RNAi. Nat Biotechnol 21, 635-637.
Jankowski, M. P., Cornuet, P. K., McIlwrath, S., Koerber, H. R., and Albers, K. M. (2006). SRY-box containing gene 11 (Sox11) transcription factor is required for neuron survival and neurite growth. Neuroscience 143, 501-514.
Junghans, D., Chauvet, S., Buhler, E., Dudley, K., Sykes, T., and Henderson, C. E. (2004). The CES-2-related transcription factor E4BP4 is an intrinsic regulator of motoneuron growth and survival. Development 131, 4425-4434.
Kuribara, R., Kinoshita, T., Miyajima, A., Shinjyo, T., Yoshihara, T., Inukai, T., Ozawa, K., Look, A. T., and Inaba, T. (1999). Two distinct interleukin-3-mediated signal pathways, Ras-NFIL3 (E4BP4) and Bcl-xL, regulate the survival of murine pro-B lymphocytes. Mol Cell Biol 19, 2754-2762.
Lee, T. I., Rinaldi, N. J., Robert, F., Odom, D. T., Bar-Joseph, Z., Gerber, G. K., Hannett, N. M., Harbison, C. T., Thompson, C. M., Simon, I., et al. (2002). Transcriptional regulatory networks in Saccharomyces cerevisiae. Science 298, 799-804.
Metzstein, M. M., Hengartner, M. O., Tsung, N., Ellis, R. E., and Horvitz, H. R. (1996). Transcriptional regulator of programmed cell death encoded by Caenorhabditis elegans gene ces-2. Nature 382, 545-547.
Mitsui, S., Yamaguchi, S., Matsuo, T., Ishida, Y., and Okamura, H. (2001). Antagonistic role of E4BP4 and PAR proteins in the circadian oscillatory mechanism. Genes Dev 15, 995-1006.
Montminy, M. R., Sevarino, K. A., Wagner, J. A., Mandel, G., and Goodman, R. H. (1986). Identification of a cyclic-AMP-responsive element within the rat somatostatin gene. Proc Natl Acad Sci USA 83, 6682-6686.
Neumann, S., and Woolf, C. J. (1999). Regeneration of dorsal column fibers into and beyond the lesion site following adult spinal cord injury. Neuron 23, 83-91.
Ozkurt, I. C., and Tetradis, S. (2003). Parathyroid hormone-induced E4BP4/NFIL3 down-regulates transcription in osteoblasts. J Biol Chem 278, 26803-26809.
Platika, D., Boulos, M. H., Balzer, L., and Fishman, M. C. (1985). Neuronal traits of clonal cell lines derived by fusion of dorsal root ganglia neurons with neuroblastoma cells. Proc Natl Acad Sci USA 82, 3499-3503.
Qiu, J., Cafferty, W. B., McMahon, S. B., and Thompson, S. W. (2005). Conditioning injury-induced spinal axon regeneration requires signal transducer and activator of transcription 3 activation. J Neurosci 25, 1645-1653.
Qiu, J., Cai, D., Dai, H., McAtee, M., Hoffman, P. N., Bregman, B. S., and Filbin, M. T. (2002). Spinal axon regeneration induced by elevation of cyclic AMP. Neuron 34, 895-903.
Raivich, G., Bohatschek, M., Da Costa, C., Iwata, O., Galiano, M., Hristova, M., Nateri, A. S., Makwana, M., Riera-Sans, L., Wolfer, D. P., et al. (2004). The AP-1 transcription factor c-Jun is required for efficient axonal regeneration. Neuron 43, 57-67.
Raivich, G., and Makwana, M. (2007). The making of successful axonal regeneration: genes, molecules and signal transduction pathways. Brain Res Rev 53, 287-311.
Riccio, A., Alvania, R. S., Lonze, B. E., Ramanan, N., Kim, T., Huang, Y., Dawson, T. M., Snyder, S. H., and Ginty, D. D. (2006). A nitric oxide signaling pathway controls CREB-mediated gene expression in neurons. Mol Cell 21, 283-294.
Seijffers, R., Allchorne, A. J., and Woolf, C. J. (2006). The transcription factor ATF-3 promotes neurite outgrowth. Mol Cell Neurosci 32, 143-154.
Seijffers, R., Mills, C. D., and Woolf, C. J. (2007). ATF3 increases the intrinsic growth state of DRG neurons to enhance peripheral nerve regeneration. J Neurosci 27, 7911-7920.
Semizarov, D., Frost, L., Sarthy, A., Kroeger, P., Halbert, D. N., and Fesik, S. W. (2003). Specificity of short interfering RNA determined through gene expression signatures. Proc Natl Acad Sci USA 100, 6347-6352.
Siegel, S., and Castellan, N. J. (1988). Nonparametric statistics for the behavioural sciences (2nd edition) (New York, McGraw-Hill).
Skene, J. H. (1989). Axonal growth-associated proteins. Annu Rev Neurosci 12, 127-156.
Skene, J. H., Jacobson, R. D., Snipes, G. J., McGuire, C. B., Norden, J. J., and Freeman, J. A. (1986). A protein induced during nerve growth (GAP-43) is a major component of growth-cone membranes. Science 233, 783-786.
Smith, D. S., and Skene, J. H. (1997). A transcription-dependent switch controls competence of adult neurons for distinct modes of axon growth. J Neurosci 17, 646-658.
Stam, F. J., MacGillavry, H. D., Armstrong, N. J., de Gunst, M. C., Zhang, Y., van Kesteren, R. E., Smit, A. B., and Verhaagen, J. (2007). Identification of candidate transcriptional modulators involved in successful regeneration after nerve injury. Eur J Neurosci 25, 3629-3637.
Tegner, J., and Bjorkegren, J. (2007). Perturbations to uncover gene networks. Trends Genet 23, 34-41.
Teng, F. Y., and Tang, B. L. (2006). Axonal regeneration in adult CNS neurons—signaling molecules and pathways. J Neurochem 96, 1501-1508.
Verge, V. M., Tetzlaff, W., Richardson, P. M., and Bisby, M. A. (1990). Correlation between GAP43 and nerve growth factor receptors in rat sensory neurons. J Neurosci 10, 926-934.
Woolf, C. J., Reynolds, M. L., Molander, C., O'Brien, C., Lindsay, R. M., and Benowitz, L. I. (1990). The growth-associated protein GAP-43 appears in dorsal root ganglion cells and in the dorsal horn of the rat spinal cord following peripheral nerve injury. Neuroscience 34, 465-478.
Yiu, G., and He, Z. (2006). Glial inhibition of CNS axon regeneration. Nat Rev Neurosci 7, 617-627.
Zhang, X., Odom, D. T., Koo, S. H., Conkright, M. D., Canettieri, G., Best, J., Chen, H., Jenner, R., Herbolsheimer, E., Jacobsen, E., et al. (2005). Genome-wide analysis of cAMP-response element binding protein occupancy, phosphorylation, and target gene activation in human tissues. Proc Natl Acad Sci USA 102, 4459-4464.

TABLE 1

| | SEQ ID No. | Accession # | |
|---|---|---|---|
| SEQ ID No. Rat | Human | Human | |
| sequence | orthologue | orthologue | Annotation human orthologue |
| 1 | 22 | NM_006941 | SRY (sex determining region Y)-box 10 SOX10 |
| 2 | 23 | NM_198723 | Transcription elongation factor A (SII), 2 TCEA2 |
| 3 | 24 | NM_003476 | Cysteine and glycine-rich protein 3 (cardiac LIM |
| | | | protein) CSRP3 |
| 4 | 25 | NM_014391 | Ankyrin repeat domain 1 (cardiac muscle) |
| | | | ANKRD1 |
| 5 | 26 | NM_032957 | Homo sapiens regulator of telomere elongation |
| | | | helicase 1 (RTEL1), |
| | | | transcript variant 2, mRNA |
| 6 | 27 | NM_030762 | Basic helix-loop-helix domain containing, class |
| | | | B, 3 BHLHB3 |
| 7 | 28 | NM_001005291 | Smith-Magenis syndrome chromosome region, |
| | | | candidate 6 SREBF1 |
| 8 | 29 | NM_203353 | PDZ and LIM domain 7 (enigma) PDLIM7 |
| 9 | 30 | NM_002166 | Inhibitor of DNA binding 2, dominant negative |
| | | | helix-loop-helix protein ID2 |
| 10 | 31 | NM_001003688 | SMAD, mothers against DPP homolog 1 |
| | | | (Drosophila) SMAD1 |
| 11 | 32 | NM_005238 | V-ets erythroblastosis virus E26 oncogene |
| | | | homolog 1 (avian) ETS1 |
| 12 | 33 | NM_005612 | RE1-silencing transcription factor REST |
| 13 | 34 | NM_005648 | Transcription elongation factor B (SIII), |
| | | | polypeptide 1 (15 kDa, elongin C) TCEB1 |
| 14 | 35 | NM_001010926 | Hairy and enhancer of split 5 (Drosophila) HES5 |
| 16 | 37 | NM_014819 | Praja 2, RING-H2 motif containing PJA2 |
| 17 | 38 | NM_003305 | Transient receptor potential cation channel, |
| | | | subfamily C, member 3 TRPC3 |
| 18 | 39 | NM_002746 | Mitogen-activated protein kinase 3 MAPK3 |
| 19 | 40 | NM_004089 | TSC22 domain family 3 DSIPI |
| 20 | 41 | NM_005955 | Metal-regulatory transcription factor 1 MTF1 |
| 21 | 42 | NM_005384 | Nuclear factor, interleukin 3 regulated NFIL3 |
| 43 | 55 | NM_005078 | Transducin-like enhancer of split 3, homolog of |
| | | | Drosophila: Tle3 |
| 44 | 58 | BG664819 | Paired-like homeodomain transcription factor |
| | | | Drg11: Prrxl1 |
| 45 | 54 | NM_003200 | Transcription factor E2a: Tcfe2a |
| 15 | 36 | NM_001674 | Activating transcription factor 3 ATF3 |
| 50 | 56 | NM_021835 | V-jun sarcoma virus 17 oncogene homolog |
| | | | (avian) |
| 51 | 52 | NM_139276 | Signal Transducer and Activator of Transcription 3 |
| | | | (acute phase response factor), STAT3 |
| 61 | 53 | NM_1334442 | CREB (corresponding protein sequence is given |
| | | | as SEQ ID NO: 57 |
| 62 | 59 | NM_003152 | Signal Transducer and Activator of Transcription |
| | | | 5a STAT5a |
| 63 | 60 | NM_001964 | Early Growth Response 1 Egr1 |

APPENDIX A
NM_006941
Hs.376984
SRY (sex determining region Y)-box 10
SOX10
||SOX10||SRY-BOX 10||SRY-RELATED HMG-BOX GENE 10||
DOMINANT MEGACOLON, MOUSE, HOMOLOG OF||SRY (sex determining region Y)-box 10||
This gene encodes a member of the SOX (SRY-related HMG-box) family of
transcription factors involved in the regulation of embryonic development and
in the determination of the cell fate. The encoded protein may act as a
transcriptional activator after forming a protein complex with other
proteins. This protein acts as a nucleocytoplasmic shuttle protein and is
important for neural crest and peripheral nervous system development..
Mutations in this gene are associated with Waardenburg-Shah and Waardenburg-
Hirschsprung disease.
DNA binding|RNA polymerase II transcription factor
activity|morphogenesis|nucleus|perception of sound|regulation of
transcription from RNA polymerase II promoter|transcription|transcription
coactivator activity
NM_002228
Hs.525704
V-jun sarcoma virus 17 oncogene homolog (avian)
JUN
||JUN||ENHANCER-BINDING PROTEIN AP1||ONCOGENE
JUN ACTIVATOR PROTEIN 1||V-JUN
AVIAN SARCOMA VIRUS 17 ONCOGENE HOMOLOG||V-jun sarcoma virus 17 oncogene
homolog (avian) ||
This gene is the putative transforming gene of avian sarcoma virus 17. It
encodes a protein which is highly similar to the viral protein, and which
interacts directly with specific target DNA sequences to regulate gene
expression. This gene is intronless and is mapped to 1p32-p31, a chromosomal
region involved in both translocations and deletions in human malignancies.
RNA polymerase II transcription factor activity|nuclear chromosome|regulation
of transcription, DNA-dependent|transcription|transcription factor
activity|transcription factor binding
NM_139276
Hs.463059
Signal transducer and activator of transcription 3 (acute-phase response factor)
STAT3
||APRF||STAT3||Signal transducer and activator of transcription
3 (acute-phase response factor) ||
The protein encoded by this gene is a member of the STAT protein family. In
response to cytokines and growth factors, STAT family members are
phosphorylated by the receptor associated kinases, and then form homo- or
heterodimers that translocate to the cell nucleus where they act as
transcription activators. This protein is activated through phosphorylation
in response to various cytokines and growth factors including IFNs, EGF, IL5,
IL6, HGF, LIF and BMP2. This protein mediates the expression of a variety of
genes in response to cell stimuli, and thus plays a key role in many cellular
processes such as cell growth and apoptosis. The small GTPase Rac1 has been
shown to bind and regulate the activity of this protein. PIAS3 protein is a
specific inhibitor of this protein. Three alternatively spliced transcript
variants encoding distinct isoforms have been described.
JAK-STAT cascade|acute-phase response|calcium ion binding|cell
motility|cytoplasm|hematopoietin/interferon-class (D200-domain) cytokine
receptor signal transducer activity|intracellular signaling cascade|negative
regulation of transcription from RNA polymerase II
promoter|neurogenesis|nucleus|nucleus|regulation of transcription, DNA-
dependent|signal transducer activity|transcription|transcription factor
activity|transcription factor activity
NM_198723
Hs.505004
Transcription elongation factor A (SII), 2
TCEA2
||TCEA2||TRANSCRIPTION ELONGATION FACTOR A, 2||transcription elongation
factor A (SII), 2||
The protein encoded by this gene is found in the nucleus, where it functions
as an SII class transcription elongation factor. Elongation factors in this
class are responsible for releasing RNA polymerase II ternary complexes from
transcriptional arrest at template-encoded arresting sites. The encoded
protein has been shown to interact with general transcription factor IIB, a
basal transcription factor. Two transcript variants encoding different
isoforms have been found for this gene.
RNA elongation|RNA elongation|defense response|nucleus|regulation of
transcription, DNA-dependent|transcription|transcription elongation factor
complex|transcription factor activity|transcriptional elongation regulator
activity
NM_003476
Hs.83577
Cysteine and glycine-rich protein 3 (cardiac LIM protein)
CSRP3
||MLP||CRP3||CSRP3||CYSTEINE-RICH PROTEIN 3||LIM DOMAIN PROTEIN,
CARDIAC||CYSTEINE- AND GLYCINE-RICH PROTEIN 3||CLP LIM DOMAIN PROTEIN,
MUSCLE||cysteine and glycine-rich protein 3 (cardiac LIM protein) ||
This gene encodes a member of the CSRP family of LIM domain proteins, which
may be involved in regulatory processes important for development and
cellular differentiation. The LIM/double zinc-finger motif found in this
protein is found in a group of proteins with critical functions in gene
regulation, cell growth, and somatic differentiation. Mutations in this gene
are thought to cause heritable forms of hypertrophic cardiomyopathy (HCM) and
dilated cardiomyopathy (DCM) in humans.
cell differentiation|myogenesis|nucleus|zinc ion binding
NM_014391
Hs.448589
Ankyrin repeat domain 1 (cardiac muscle)
ANKRD1
||ANKRD1||Ankyrin repeat domain 1 (cardiac muscle) ||
The protein encoded by this gene is localized to the nucleus of endothelial
cells and is induced by IL-1 and TNF-alpha stimulation. Studies in rat
cardiomyocytes suggest that this gene functions as a transcription factor.
DNA binding|defense response|nucleus|signal transduction
NM_032957
Hs.434878
Homo sapiens regulator of telomere elongation helicase 1
RTEL1
||RTEL1||Homo sapiens regulator of telomere elongation helicase 1||
NM_030762
Hs.177841
Basic helix-loop-helix domain containing, class B, 3
BHLHB3
||DEC2||BHLHB3||SHARP1, RAT, HOMOLOG OF||basic helix-loop-helix domain
containing, class B, 3||BASIC HELIX-LOOP-HELIX DOMAIN-CONTAINING PROTEIN,
CLASS B, 3||
cell differentiation|cell proliferation|nucleus|organogenesis|regulation of
transcription, DNA-dependent|transcription|transcription factor activity
NM_001005291
Hs.190284
Smith-Magenis syndrome chromosome region, candidate 6
SREBF1
||||SREBF1||Smith-Magenis syndrome chromosome region, candidate 6||
This gene encodes a transcription factor that binds to the sterol regulatory
element-1 (SRE1), which is a decamer flanking the low density lipoprotein
receptor gene and some genes involved in sterol biosynthesis. The protein is
synthesized as a precursor that is attached to the nuclear membrane and
endoplasmic reticulum. Following cleavage, the mature protein translocates to
the nucleus and activates transcription by binding to the SRE1. Sterols
inhibit the cleavage of the precursor, and the mature nuclear form is rapidly
catabolized, thereby reducing transcription. The protein is a member of the
basic helix-loop-helix-leucine zipper (bHLH-Zip) transcription factor family.
This gene is located within the Smith-Magenis syndrome region on chromosome
17. Two transcript variants encoding different isoforms have been found for
this gene.
Golgi apparatus|RNA polymerase II transcription factor activity|cholesterol
metabolism|endoplasmic reticulum membrane|integral to membrane|lipid
metabolism|nuclear membrane|regulation of transcription from RNA polymerase
II promoter|steroid metabolism|transcription|transcription factor activity
NM_203353
Hs.533040
PDZ and LIM domain 7 (enigma)
PDLIM7
||LMP1||PDLIM7||LIM DOMAIN PROTEIN ENIGMA||LIM MINERALIZATION PROTEIN 1||PDZ
and LIM domain 7 (enigma) ||
The protein encoded by this gene is representative of a family of proteins
composed of conserved PDZ and LIM domains. LIM domains are proposed to
function in protein-protein recognition in a variety of contexts including
gene transcription and development and in cytoskeletal interaction. The LIM
domains of this protein bind to protein kinases, whereas the PDZ domain binds
to actin filaments. The gene product is involved in the assembly of an actin
filament-associated complex essential for transmission of ret/ptc2 mitogenic
signaling. The biological function is likely to be that of an adapter, with
the PDZ domain localizing the LIM-binding proteins to actin filaments of both
skeletal muscle and nonmuscle tissues. Alternative splicing of this gene
results in multiple transcript variants.
protein binding|receptor mediated endocytosis|zinc ion binding
NM_002166
Hs.180919
Inhibitor of DNA binding 2, dominant negative helix-loop-helix protein
ID2
||ID2||INHIBITOR OF DIFFERENTIATION 2||inhibitor of DNA binding 2,
dominant negative helix-loop-helix protein||
The protein encoded by this gene belongs to the inhibitor of DNA binding (ID)
family, members of which are transcriptional regulators that contain a helix-
loop-helix (HLH) domain but not a basic domain. Members of the ID family
inhibit the functions of basic helix-loop-helix transcription factors in a
dominant-negative manner by suppressing their heterodimerization partners
through the HLH domains. This protein may play a role in negatively
regulating cell differentiation. A pseudogene has been identified for this
gene.
development|nucleus
NM_001003688
Hs.549050
SMAD, mothers against DPP homolog 1 (Drosophila)
SMAD1
||MADH1||SMAD1||MADR1||BSP1||TGF-BETA
SIGNALING PROTEIN 1||MAD, DROSOPHILA, HOMOLOG OF||SMA- AND MAD-RELATED
PROTEIN 1||MOTHERS AGAINST DECAPENTAPLEGIC, DROSOPHILA, HOMOLOG OF,
1||SMAD, mothers against DPP homolog 1 (Drosophila)||
The protein encoded by this gene belongs to the SMAD, a family of proteins
similar to the gene products of the Drosophila gene ‘mothers against
decapentaplegic’ (Mad) and the C. elegans gene Sma. SMAD proteins are signal
transducers and transcriptional modulators that mediate multiple signaling
pathways. This protein mediates the signals of the bone morphogenetic
proteins (BMPs), which are involved in a range of biological activities
including cell growth, apoptosis, morphogenesis, development and immune
responses. In response to BMP ligands, this protein can be phosphorylated and
activated by the BMP receptor kinase. The phosphorylated form of this protein
forms a complex with SMAD4, which is important for its function in the
transcription regulation. This protein is a target for SMAD-specific E3
ubiquitin ligases, such as SMURF1 and SMURF2, and undergoes ubiquitination
and proteasome-mediated degradation. Alternatively spliced transcript
variants encoding the same protein have been observed.
BMP signaling pathway|embryonic pattern specification|integral to
membrane|integral to membrane|nucleus|nucleus|receptor signaling protein
activity|receptor signaling protein activity|regulation of transcription,
DNA-dependent|signal transduction|signal
transduction|transcription|transcription factor activity|transcriptional
activator activity|transcriptional activator activity|transforming growth
factor beta receptor signaling pathway|transforming growth factor beta
receptor signaling pathway
NM_005238
Hs.369438
V-ets erythroblastosis virus E26 oncogene homolog 1 (avian)
ETS1
||ETS1||EWSR2||ETS-1||ONCOGENE ETS1||ETS1 ONCOGENE||ets
protein||Avian erythroblastosis virus E26 (v-ets) oncogene homolog-1||v-ets
avian erythroblastosis virus E2 oncogene homolog 1||v-ets avian erythroblastosis
virus E26 oncogene homolog 1||v-ets erythroblastosis virus E26 oncogene homolog 1
(avian) ||v-ets erythroblastosis virus E26 oncogene homolog 1 (avian)||
RNA polymerase II transcription factor activity|immune response|negative
regulation of cell proliferation|nucleus|regulation of transcription,
DNA-dependent|transcription|transcription factor activity|transcription
from RNA polymerase II promoter
NM_005612
Hs.401145
RE1-silencing transcription factor
REST
||NRSF||REST||NEURON-RESTRICTIVE SILENCER FACTOR||RE1-silencing
transcription factor||
This gene encodes a transcriptional represser which represses neuronal genes
in non-neuronal tissues. It is a member of the Kruppel-type zinc finger
transcription factor family. It represses transcription by binding a DNA
sequence element called the neuron-restrictive silencer element. The protein
is also found in undifferentiated neuronal progenitor cells, and it is
thought that this represser may act as a master negative regular of
neurogenesis. Alternatively spliced transcript variants have been described;
however, their full length nature has not been determined.
nucleic acid binding|nucleus|regulation of transcription, DNA-
dependent|transcriptional represser activity|zinc ion binding
NM_005648
Hs.546305
Transcription elongation factor B (SIII), polypeptide 1 (15 kDa, elongin C)
TCEB1
||TCEB1||ELONGIN, 15-KD SUBUNIT||TRANSCRIPTION ELONGATION FACTOR B,
1||transcription elongation factor B (SIII), polypeptide 1 (15 kDa, elogin C) ||
This gene encodes the protein elongin C, which is a subunit of the
transcription factor B (SIII) complex. The SIII complex is composed of
elongins A/A2, B and C. It activates elongation by RNA polymerase II by
suppressing transient pausing of the polymerase at many sites within
transcription units. Elongin A functions as the transcriptionally active
component of the SIII complex, whereas elongins B and C are regulatory
subunits. Elongin A2 is specifically expressed in the testis, and capable of
forming a stable complex with elongins B and C. The von Hippel-Lindau tumor
suppressor protein binds to elongins B and C, and thereby inhibits
transcription elongation.
nucleus|protein binding|regulation of transcription from RNA polymerase II
promoter|transcription|transcriptional elongation regulator activity|ubiquitin cycle
NM_001010926
Hs.57971
Hairy and enhancer of split 5 (Drosophila)
HES5
||HES5||HAIRY/ENHANCER OF SPLIT, DROSOPHILA, HOMOLOG OF, 5||hairy and
enhancer of split 5 (Drosophila) ||
DNA binding|regulation of transcription, DNA-dependent
NM_001674
Hs.460
Activating transcription factor 3
ATF3
||ATF3||ATF3deltaZip3||ATF3deltaZip2c||Activating transcription
factor 3||activating transcription factor 3 long isoform||activating
transcription factor 3 delta Zip isoform||
Activating transcription factor 3 (ATF3) is a member of the mammalian
activation transcription factor/cAMP responsive element-binding (CREB)
protein family of transcription factors. It encodes a protein with a
calculated molecular mass of 22 kD. ATF3 represses rather than activates
transcription from promoters with ATF binding elements. An alternatively
spliced form of ATF3 (ATF3 delta Zip) encodes a truncated form ATF3 protein
lacking the leucine zipper protein-dimerization motif and does not bind to
DNA. In contrast to ATF3, ATF3 delta Zip stimulates transcription presumably
by sequestering inhibitory co-factors away from the promoter. It is possible
that alternative splicing of the ATF3 gene may be physiologically important
in the regulation of target genes.
DNA binding|nucleus|regulation of transcription, DNA-
dependent|transcription|transcription corepressor activity|transcription
factor activity
NM_005078
Hs.287362
Transducin-like enhancer of split 3 (E(sp1) homolog, Drosophila)
TLE3
||ESG3||TLE3||ENHANCER OF SPLIT GROUCHO 3||transducin-like
enhancer of split 3 (E(sp1) homolog, Drosophila)||frizzled
signaling pathway|nucleus|organogenesis|regulation of transcription,
DNA-dependent|signal transduction
NM_014819
Hs.483036
Praja 2, RING-H2 motif containing
PJA2
||PJA2||praja 2, RING-H2 motif containing||
protein ubiquitination|ubiquitin ligase complex|ubiquitin-protein ligase
activity|zinc ion binding
NM_003305
Hs.150981
Transient receptor potential cation channel, subfamily C, member 3
TRPC3
||TRPC3||TRP3||TRANSIENT RECEPTOR POTENTIAL CHANNEL 3||TRANSIENT
RECEPTOR POTENTIAL, DROSOPHILA, HOMOLOG OF, 3||transient receptor
potential cation channel, subfamily C, member 3||calcium ion transport|cation
transport|integral to plasma membrane|membrane|phototransduction|store-operated
calcium channel activity
NM_002746
Hs.861
Mitogen-activated protein kinase 3
MAPK3
||MAPK3||p44ERK1||PRKM3||p44MAPK||EXTRACELLULAR SIGNAL-REGULATED
KINASE 1||Mitogen-activated protein kinase 3 ||PROTEIN KINASE, MITOGEN-ACTIVATED,
3|| ATP binding|ATP binding |MAP kinase activity|MAP kinase
activity|cellular_component unknown| protein amino acid phosphorylation|protein
amino acid phosphorylation|protein serine/threonine kinase activity|regulation of
cell cycle|transferase activity
NM_004089
Hs.522074
TSC22 domain family 3
DSIPI
||GILZ||TSC-22R||DKFZp313A1123||hDIP||DSIPI||TSC22D3||TSC-22
related protein|glucocorticoid-induced leucine zipper protein||TSC22 domain family
3||DELTA SLEEP-INDUCING PEPTIDE, IMMUNOREACTOR||DSIP-immunoreactive leucine zipper
protein||delta sleep inducing peptide, immunoreactor||TSC22 domain family 3
isoform 1||TSC22 domain family 3 isoform 3||TSC22 domain family 3 isoform 2||
The protein encoded by this gene shares significant sequence identity with
the murine TSC-22 and Drosophila shs, both of which are leucine zipper
proteins, that function as transcriptional regulators. The expression of this
gene is stimulated by glucocorticoids and interleukin 10, and it appears to
play a key role in the anti-inflammatory and immunosuppressive effects of
this steroid and chemokine. Transcript variants encoding different isoforms
have been identified for this gene.
regulation of transcription, DNA-dependent|transcription factor activity
NM_005955
Hs.471991
Metal-regulatory transcription factor 1
MTF1
||MTF1||Metal-regulatory transcription factor 1||
nucleus|regulation of transcription from RNA polymerase II promote|response
to metal ion|transcription coactivator activity|transcription factor
activity|zinc ion binding
NM_005384
Hs.79334
Nuclear factor, interleukin 3 regulated
NFIL3
||E4BP4||NFIL3A||NFIL3||NUCLEAR FACTOR,
INTERLEUKIN 3-REGULATED||Nuclear factor, interleukin 3 regulated||
immune response|nucleus|regulation of transcription, DNA-
dependent|transcription corepressor activity|transcription factor
activity|transcription from RNA polymerase II promoter
DRGX
NM_001080520
Hs.534530
Dorsal root ganglia homeobox
||||DRGX||Dorsal root ganglia homeobox||is also named Prrxl1
TCF3
N_003200
Hs.371282
Transcription factor 3 (E2A immunoglobulin enhancer binding factors E12/E47)
is also named Tcfe2a
||TCF3||E2A/TFPT FUSION GENE||E2A/PBX1 FUSION GENE||
IMMUNOGLOBULIN ENHANCER-BINDING FACTORS E12/E47||ITF1 E2A/HLF FUSION GENE||
IMMUNOGLOBULIN TRANSCRIPTION FACTOR 1||transcription factor 3 (E2A immunoglobulin enhancer
binding factors E12/E47)||
nucleus|regulation of transcription, DNA-dependent|transcription|transcription
factor activity
CREB1
NM_134442
Hs. 584750
CAMP responsive element binding protein 1
||CREB1||MGC9284||transactivator protein||cAMP-response
element-binding protein-1||active transcription factor CREB||cAMP RESPONSE
ELEMENT-BINDING PROTEIN 1||cAMP responsive element binding protein 1||cAMP
responsive element binding protein 1 isoform B||cAMP responsive element binding protein 1
isoform A||
This gene encodes a transcription factor that is a member of the leucine
zipper family of DNA binding proteins. This protein binds as a homodimer to
the cAMP-responsive element, an octameric palindrome. The protein is
phosphorylated by several protein kinases, and induces transcription of genes
in response to hormonal stimulation of the cAMP pathway. Alternate splicing of
this gene results in two transcript variants encoding different isoforms.
DNA binding|nucleus|nucleus|protein binding|regulation of transcription,
DNA-dependent|signal transduction|transcription cofactor activity|
transcription factor activity

1. A method for promoting generation or regeneration of a neuronal cell, the method comprising the step of altering the activity or the steady state level of a polypeptide in the neuronal cell, wherein the polypeptide is selected from a NFIL3, BHLHB3, ETS1, TRPC3, REST, PJA2, MTF1, TCEA2, PRRXL1, TCEB1, PDLIM7, ID2, TLE3, MAPK3, ANKRD1, SOX10, HES5, SREBF1, SMAD1, RTEL1, TCFE2A, CSRP3, STAT5a, Egr1 and a TSC22D3.

2. A method according to claim 1, wherein regeneration of the neuronal cell is promoted by:
increasing the activity or the steady-state level of a polypeptide selected from: a BHLHB3, ETS1, TRPC3, REST, PJA2, MTF1, TCEA2, PRRXL1, TCEB1, PDLIM7, ID2, TLE3, MAPK3 and a ANKRD1 and/or
decreasing the activity or the steady-state level of a polypeptide selected from: a SOX10, HES5, SREBF1, SMAD1, RTEL1, TCFE2A, CSRP3, TSC22D3, STAT5a, Egr1 and a NFIL3.

3. A method according to claim 2, wherein regeneration of the neuronal cell is promoted by at least decreasing the activity or the steady-state level of a NFIL3.

4. A method according to claim 2, wherein the activity or the steady-state level of the polypeptide is increased by introducing an nucleic acid construct into the neuronal cell, wherein the nucleic acid construct comprises a nucleotide sequence encoding the polypeptide, and wherein the nucleotide sequence is under control of a promoter capable of driving expression of the nucleotide sequence in the neuronal cell.

5. A method according to claim 2, wherein the activity or the steady-state level of the polypeptide is decreased by introducing:
a nucleic acid construct into the neuronal cell, wherein the nucleic acid construct comprises an antisense nucleotide sequence that is capable of inhibiting the expression of the nucleotide sequence encoding the polypeptide, and wherein, optionally, the antisense nucleotide sequence is under control of a promoter capable of driving expression of the antisense nucleotide sequence in the neuronal cell and/or
a nucleic acid construct into the neuronal cell, wherein the nucleic acid construct comprises a dominant negative nucleotide sequence that is capable of inhibiting the activity of the polypeptide, and wherein, optionally, the dominant negative nucleotide sequence is under the control of a promoter capable of driving expression of the dominant negative nucleotide sequence in the neuronal cell.

6. A method according to claim 5, wherein the dominant negative nucleotide sequence is a dominant negative nucleotide sequence encoding a dominant negative NFIL3, preferably an A-NFIL3 or a RD-NFIL3.

7. A method according to claim 4, wherein the promoter is a neuronal cell specific promoter.

8. A method for treating a neurotraumatic injury or a neurodegenerative disease in a subject, the method comprising pharmacologically altering the activity or the steady-state level of a polypeptide as defined in claim 1, in an injured neuron in the subject, the alteration being sufficient to of inducing generation or regeneration of the injured or degenerated neuron, preferably axonal generation or regeneration of the injured or degenerated neuron.

9. A method according to claim 8, wherein the method comprises the step of administering to the subject a therapeutically effective amount of a pharmaceutical composition comprising a nucleic acid construct as defined in claims 4 and/or 5 and wherein preferably the pharmaceutical composition is administered at a site of neuronal injury or degeneration.

10. A nucleic acid construct comprising a nucleotide sequence encoding a polypeptide that comprises an amino acid sequence that is encoded by a nucleotide sequence selected from:
(a) a nucleotide sequence that has at least 80% identity with a sequence selected from SEQ ID NO.'s 1-45, 46, 48, 50-56, 58-63; and,
(b) a nucleotide sequence that encodes an amino acid sequence that has at least 80% amino acid identity with an amino acid sequence encoded by a nucleotide sequence selected from SEQ ID NO.'s 1-45, 46, 48, 50-56, 58-63
wherein the nucleotide sequence is operably linked to a promoter that is capable of driving expression of the nucleotide sequence in the neuronal cell.

11. A nucleic acid construct comprising a nucleotide sequence encoding an RNAi agent that is capable of inhibiting the expression of a polypeptide that comprises an amino acid sequence that is encoded by a nucleotide sequence selected from:
(a) a nucleotide sequence that has at least 80% identity with a sequence selected from SEQ ID NO.'s 1-45, 54-55, 58-63; and,
(b) a nucleotide sequence that encodes an amino acid sequence that has at least 80% amino acid identity with an amino acid sequence encoded by a nucleotide sequence selected from SEQ ID NO.'s 1-45, 54-55, 58-63,
wherein optionally the nucleotide sequence encoding the RNAi agent is operably linked to a promoter that is capable of driving expression of the nucleotide sequence in the neuronal cell.

12. A method for diagnosing the status of generation or regeneration of a neuron in a subject, the method comprising the steps of:
(a) determining the expression level of a nucleotide sequence encoding a polypeptide as identified in claim 1 in the subject's generating or regenerating neuron; and,
(b) comparing the expression level of the nucleotide sequence with a reference value for expression level of the nucleotide sequence, the reference value preferably being the average value for the expression level in a neuron of healthy individuals.

13. A nucleotide sequence as defined in claim 10.

14. A nucleotide sequence as defined in claim 11.


SEQUENCE LISTING (63 sequences)

SEQ ID NO: 1 - DNA, 3030 bp, rattus norvegicus
cggacactagactgtagaggtgagaccaaggataggagccctcgggcaacgggaggaagaacatagaatagccttgaagggcagaaaagggggagggggtcttttctcagtggggaagagtgactgagggacttcactacatccagggctgagaccctttcccaggagaggaagagctcagcatggaagtgagacccctccccccaacccaatgtcctcctcacccttcttgaagagtacccactcccctccccctccttccttccccagccgtctgcagcggctcagtcgctcagtcagtctcgggctgtccagccagggtgtttggtggtgaggattcaggctccgtccagacaaggcagtggcctgaggctcagggccccccagcccctccctcccagtccatcagcgtcactccccagccccgagctggaccgcacaccttgggacacggttttccacttcctcaggacgagccccagactggaggagaggtcggaggaggtgggcgttgggctcttcacgaggaccccggcggcgggcccgggggaggcggccgaagcggcggcggccgggagcgacatggccgaggagcaagacctatcagaggtggagctgagccccgtgggctcggaggaacctcgctgcctgtccccaagcagcgcgccgtcgctgggacccgacggcggcggcggtggctcaggcctgcgagccagcccagggcccggcgaattgggcaaggtcaagaaggaacagcaggacggcgaggcagacgatgacaagttcccggtgtgcatccgcgaggcggtcagccaggtgctcagcggctacgactggacgctggtgcccatgcccgtgcgggtcaacggtgccagcaagagcaagccgcacgtcaaacggcccatgaacgcctttatggtgtgggcacaggcggcacgcagaaagttagccgaccagtacccgcacctccacaatgctgagctcagcaagacgctgggcaagctctggaggttgctgaacgagagtgacaagcgccccttcattgaggaggccgagcggctccggatgcagcacaaaaaagaccatccggactacaagtaccaacctcggcggcggaagaatgggaaggcagcccagggggaggcagagtgcccaggcggggagaccgaccaaggaggggctgctgctatccaggctcactacaagagtgcccacctggaccaccggcacccagaagaaggctcccccatgtcagatgggaacccagagcacccctcaggccagagccatggccctcctacccctccaaccaccccaaagacagagctgcagtccggcaaggcagatcccaaaagggatgggcgctccttgggggagggcgggaagccccacatcgacttcggcaatgtggacatcggggagatcagccatgaggtaatgtccaacatggagacctttgatgtgactgagctggaccaatacctgccgcccaatgggcacccaggccacgtgggcagctactcggcagctggctacgggctgagcagtgccctggctgtggccagtggacactctgcctggatctccaagccaccaggtgtggctctgcccacggtctcgccccctgctgtggatgccaaagcccaggtgaagacagagaccacagggccccagggacccccacactacaccgaccagccgtccacttcccagatcgcctacacctccctcagtctgccccactacggctccgccttcccctccatctcacgcccccagtttgactactctgaccatcagccctcaggaccctattatggccacgcaggccaggcctctggcctctactcagctttttcctacatgggcccctctcagcggcccctctacacggccatctctgaccccagcccctcagggccccagtcccacagtcccacacactgggaacagccagtatatacgactctatcccgaccttagaggggccctgtcaccattggtgcccacgtggcccagggccccttcccccagcctgtgtgccctgctcctcatcagcctcatgcccagcagtggcagtggaggaggctgcagaggctgacagcagcaggcaggcttctgccagctcctgccctgatgacctccacccaccctggctcgggaggccaagccctgactgagctggcaaaggaggagtttggtggccatcccagactgagggtgaggggccacccatccccagaaataaccctctatcccaggacctgagaagtcgttctcaagtctttcagggaggcagaaaagggtatggtgggaaacctcactgctcctgttctctcctgggagagcaaggggctcaagggcctgtgtgccactgtgtcactgtgcctaaaagctaggctgcctagggacctgcttcaaagcctggagctggctctgtcctgtcactaaggactcactgaggacagctttgaacagctttgtgaagagcagggtgggacactcaatggacactctgatcctttcacccccctcctcacctgaatgggaactggccgaggtccccagatccagttctgcattagtaacctcatccctcacctaactgaggagagtcctcatgttcttccccacgacctccactcctcaaggcccaccccaggtgaggaggcaggggctccaggaaagaatcagaggtgtccagccttcctctcgccttctgtcagcctcagttagcactgcacgggccctagccccagagcccagtacctgggttgctggcagcaggctggacactaaaccccttccatgtacagtctacctttgcctcgcaccctttgctccagcgatatcttaataaagtacgtagctctgtctccccatctcctgtctgcagcccagtccacacgtaacacatcgctgcccctttatctccacagtcaccatgttcccatccctctagctactccagccccaattaacttgcattaagcactccaagtaataaaatcaaagcttctggttgcctgc

SEQ ID NO: 2 - DNA, 310 bp, rattus norvegicus
  misc_feature (282)..(282): n is a, c, g, or t
ttttttttttttttttttgaaggcaaaaaagaaacatttaatgatgaggggcaaagggcaggcaggtgcacaccaggcaggctggaacggggcatgccacctgcagggtatctccagagaagctgtgtccaccaagagcgttgcaggaggttcagggttagccatggccagggctacatcagtacaaggactcagcagaacttccatcgattcccacactcattgcagacaacataggtggtcatgggctcatcagagctacgggtctgcacctgcgtgtangtgcagttcttcttcctgcacttgttgc

SEQ ID NO: 3 - DNA, 853 bp, rattus norvegicus
tcatctcggcagattgactttggcccctgagtcttcaccatgccgaactggggtggaggtgcaaaatgtggagcctgcgacaagacggtttaccatgcagaagaaatccagtgcaatgggaggagcttccataagacctgtttccactgcatggcctgcaggaaggctctggacagcaccacagtggcagctcatgagtcagagatctactgtaaggtctgctatgggcgcaagtatggccccaaggggatcgggtttggacaaggcgctggctgcctcagcacagacaccggcgagcaccttggcctgcagttccaacaatccccaaagccagcacgtgcagccaccacgagcaacccttccaagttctctgcgaagtttggagaatcagagaagtgcccacgatgcggaaagtcggtatatgctgctgagaaggtcatgggaggtggcaagccctggcataagacctgctttccgtgcgccatctgtgggaagagcctggagtccacaaatgtcactgacaaggatggggagctttattgcaaagtttgctatgccaaaaattttggccccacaggcattgggtttggagggcttacacaccaagtggaaaagaaggagtgaaacggcgtgctttctcacagccctgctagtcaacagcagggcttagtgaccccatagatgtaactttggccccgcagcctcctttgtagctggactgcatttctcttcagaagcagtcataggctgcactgaccgtaaaaagagaccaccggaaatgaatctgcaaaagatgcttcattatttactatttttggggtgggagagacaataaacgttttaatggtaaaaa

SEQ ID NO: 4 - DNA, 1749 bp, rattus norvegicus
cccgggcaggtccaggggttcagccacaagagggaaaaacatagactaacggctgccaacatgatggtttttcgagtagaggagctggtaacgggcaaaaagaacagcaatgggtcctcaggggagttccttcctggcgagttcagaaatggagagtatgaagctgctgttgccttggagaaacaagaggacttgaagacacttccagccaacagtgtgaacctgggggaagaacaacggaaaagtgagaaagttcgagaggcagagctcaaaaagaaaaaactagaacaaagatcaaagcttgaaaacttagaagaccttgaaataattgttcaactgaagaaaagaaaaaaatacaagaaaactaaagttccagttgtgaaggaaccggagcctgaaattattactgaacctgtggatgtgccaaggtttctgaaagctgcactggagaacaaactgccagttgtagagaaattcctgtcagacaagaacagccccgacgtctgcgatgagtataaacggaccgctctccatagagcatgcttagaaggacacttggcgatcgtggagaagttaatggaggctggagcccagattgaattccgagatatgctggaatccacagccatccactgggcatgtcgtggaggaaacctggatgtcctgaaactgttgctaaacaaaggtgccaaaatcagtgcccgagacaagctgctcagcacagcgctgcacgtggcggtgaggaccggtcactacgagtgtgctgagcacctcatcgcctgcgaggcggatctcaatgccaaggacagagaaggagacacccctctgcatgatgcggtgaggttgaatcgctacaagatgatccggctcttgatgaccttcggtgcggacctcaatgtcaagaactgtgctgggaagatccctatggatctggtgttgcactggcagaatggaaccaaagcgatattcgacagcctcaaggagaatgcctacaaaaactcgcgcatagctacgttctgaggaaagagactcgacaggagctgttctcagccgttttcaaaacatttcccagcagcggagcaaccagctataacacccagcttttgtttgccagccaaatagggagataagctttaaagacgcttcatggaaggaccctgggttagagatgctaacatgtcctctagcgatgctcaccatttttatctgctcctatttatttatttatttatttcatagcactggtagtcaggttttcatgagcatcattttgggggagcaagacttaatctgtgtgtaacattcggagtcttcccgtggctctaaaaccgcgtgggtgaatggtgtctaatctcccttttgtgtcctcacttgcattaaagctactcaaggtatttttgaaagccaagggttggtcggtcgctgcagcccttctctggattctcactgctgatatccgaatgaagcagcagaaggtgtacaggaatgcagaaggggatgggagcagttctgcaggacctaagccgtggctcgccaggcacccacaggtgtggtttgccggatcacacagtgccgaggtgaaggccacgtcaggagcgtgtgtggagggggatggagggagtaggcaatggggcctcactaacctgaggtcgatgagaggaaaaagtccaaaaggacatgccctgtaaatgtgtaaatggtattgtgttgtatgcatattttatattcctaataaatgcaaactaattc

SEQ ID NO: 5 - DNA, 623 bp, rattus norvegicus
ttttttttttttttttttaatgttggtaattcctactaagccttgaaaattgaaaacagaccccacttccgggtgaagcgctcagacaaaaccccacgaggacactatttgtctccaacgacagacagcacttgggatcatgaggttagcacctatccctcaccttccaggtttggtgactcctctgtcatccccaggcagctgaacagcatcaatagcctcctccaggtgcagcaggatcatcttcaactttgcgatgtcttccagctccatgttgagacctgagctggacgtgtctataatgaactcctgttggagttcaccatgctgagccacccgggcttgctcctccaaaacctggttgataacctccagtcctgaagccacatctcggggagtcaagtcaaaggaggctgactcctcacatatcttttccacattgtgagcttcatcaaagattacaaccgttcccttcaggtcaatgttgtgtgccttacgactctgtaagtgtgcagagagaaatcgtgacatccgcctccatcttttctaggctgctcttcccctcttgctccggtctcctctcctctctaaaacttttctcctgcccatccttccttctcgtt

SEQ ID NO: 6 - DNA, 3101 bp, rattus norvegicus
tgtctacaccataattcctttgtctttgagccagctcacaaatgtcactgtggttctgagtgtgggggtcttggtgcagtccctcccctcccagtcccttccgtcgaggagcatggtgctagtgctgccacagcctggagacgcacacaaccccccaaaatctctccagacgaccgtcccacgatcacaggacagaaccctccaaatcgaaacggaggaaacggacagccattgaacatggacgaaggaatccctcatttgcaagagagacagttactggaacatagggattttataggactggactattcctctttgtatatgtgtaaacccaaaaggagcttgaagcgagacgacaccaaggatacctacaaattaccgcacagattaatagaaaagaagagacgagaccgaattaatgaatgtattgctcagctgaaagacttactgcctgaacatctgaaattgacaacactggggcatctggagaaagcagtagtcttggaattaactttgaagcacttaaaagctttaacagccttaacggagcagcagcatcagaagataattgctttacagaatggggagcgctctctgaaatcgccggtccaggccgacttggatgcgttccactcggggtttcaaacctgcgccaaagaagtcttgcaatacctcgcgcgctttgagagctggacgcccagggaaccgcgctgcgcacagctcgtcagccacctgcacgccgtggctacccagcttctgacgccacaggtgaccccaggcaggggccctgggcgcgcgccctgcagcgctggggctgcagccgcctccggttccgagcgcgtcgcccgctgcgtgccggtcatccagcggactcagcccggcacggagcccgagcacgacacggacaccgacagcggctatggaggcgaggcggagcagggccgcgccgccgtcaagcaggagccacccggggacccgtcgctgcgcccaagaggctgaagctggaggcgcgcggcgcgctcctgggcccggagcccgcgctgctcggctctctcgtggcgttgggcgggggtgcgcccttcgcgcagcccgccgccgcgcccttctgcctgcccttctacctgctgtcgccgtccgccgccgcctacgtacagccctggctagacaagagtggcctggacaagtatctgtaccccgcggcggccgcgcccttcccgctgctgtatcccggcatccccgcagcagccgccgctgccgccgccgccgctttcccttgcttgtcgtccgtgctatcgccacccccggagaaggcaggttcggccgctggtgccccattcctggcgcacgaggtggcgcccccggggtcgctgcgcccccagcacgcgcatagccgcacccacctgccgcacgccgtgaacccagagagctctcaggaagatgccacgcagccggccaaggacgccccctgaacccagcattccttccagaacagggcagggggctcctgaggagtcgccaggtttccaagttcaaacatcccctaaggcgtaccagggaggaagagtaagagatgctctgctcgacaggcttaggacaaaaacaggtgttttgtgtatgtttggagttcctgttttgcccctttctcacccttctgccaccccaccctctaccctttgacactcccttccccatccctgctgtcacagagcctccctgagaaatactggttatcttaaattaccctcccttacatttagttcacgtcctctgtttccaaacatagaccctggttcaggagtctgttgggtgggagagccacacggaaccagttagagtgcctggtatcagggctccttgacccaggcctggaacagtagctgtgtcccctgtctgtccccttaggaggtgacccataactgagggtctctgaaagttacattgacgtgtcagtattttgtattcttcagctttttggaaggtacctctttttcaaagaagtgaggatgccattgccctgttgtgaggtggctggagtggtgtctttataccttgcacctgttgggagaaactgagagttggggccatcttcaggcactgtgtcagtgtgggagctggaagagggagtttggagcccgtggcgcctttctcgcactttattgacaaattgacctcaacccctttgtcccatgtctcaactcacagatatatgtcataggttatatatttgtgtttctgatccctcgttattttatccatcatggtcccaaatttttgtaatgttactggggtttggggtggggtggggtgttaaagtgctctgggctggaaaaagacaagcccaaacctattgattgtcgaattcttagatgacagaagtggagagaggggcttgtggtcccttgtgatgggaagtgctgtgaacatgtagaaggccctgccagcctcgctctctcaagtctgtatgtatttttcgggagaccaaaccagacaccagataatcaggaagaaagctttttaaaataaggcaaaaaccgagaccttgtctagatatttttagtttgttgccaaggtagcactgagaaatctcacttgaatgttacataaggagtgattcacaatagtctagagtgaagaaagttatctgggtctgtgagtgttcgggtccgtttgctgctgctgttgctactgtttgcctcaaacgctgtgtttaaacaacgttaaacttcttagcctaccaaggcggccgtatgtacatagctgttaatacccccaactaatgtctgacatgctatttttgtagggagaagatacctgctagtgatattttgagttaaaatatcttttggggcggacttggtgaaatgtttgcactttggtcacaatgcttctactgcttggtgcaacgttacgctgtcttaaattattaaacaaataaaaaatactatctgcaagaaaaaccagctggtttagacaagtttagtatgtaaagataagctagaaactatctttatattctagtattttcagcactccatattacctaaatattgccacactattttgtgatttaaaagttcttactaaggaataaaatctttata

SEQ ID NO: 7 - DNA, 2972 bp, rattus norvegicus
ggagccatggattgcacatttgaagacatgcttcagctcatcaacaaccaagacagtgacttccctggcctatttgatgccccctatgctgggggtgagacaggagacacaggccccagcagccctggtgccagctctcctgagagcttctcttctcctgcttctctgggctcctctctggaagccttcctgggaggacccaaggtgacacctgcacccttgtcccctccaccatcggcacccactgctgtaaagatgtacccgtccgtgccccccttctcccctgggcctggaatcaaagaggagccagtgccactcaccatcctgcagcccccagcaccacagccatcgccagggaccctgttgcctccgagcttccctcctccacctgtgcagctcagccctgctcctgtgctggggtactcaagcctgccttccggcttctcaggaacccttcctgggaacacccagcagacgccatctagcctgccactgggctccacgccaggaatctcgcccacccccttacacacccaggtccagagctcggccgcccagcagccgccgccagcctcagcagcccctagaatgagcactgtggcctcacagatccagcaggtccccgttgtactgcagccacacttcatcaaggcagactcgctgctgctgacagctgtaaagacagacacaggagccacaatgaagaccgcaggcatcaacaccctggctcctggcacagccgtgcaggcaggccccttgcagaccctggtgagtggagggaccatcctggccacagtaccactggttgtggacacagacaaactgcccatccaccgactagcagctggtggcaaggccctgggctcagctcagagccgtggtgagaagcgcacagcccacaatgccattgagaagcgctaccgttcctctatcaatgacaagattgtggagctcaaggacctggtggtgggcactgaggcaaagctgaataaatctgctgtcttgcgcaaggccatcgactacatccgcttcttacagcacagcaaccagaaactcaagcaggagaacctgaccctgcgaagtgctcacaaaagcaaatcactgaaagacctggtgtcagcttgtggcagtggaggaggcacagatgtgtctatggagggcatgaaacctgaagtggtagaaacgctgacccctccaccctcagacgccggctcaccctcccagagtagccccttgtccttgggcagcagaggcagcagcagtggtggcagtgactctgagcccgacagcccagcctttgaggataaccaggtgaaagcccagcggctgccttcacatagccgaggcatgctggaccgctcccgcctggccctgtgtgtactggtcttcctgtgtctgacctgcaacccattggcctcactgtttggctggggcatcctcactccctctgatgcttcgggtgtgcaccgtagttctgggcgcagcatgctggaggccgagagcagagatggctctaattggacccagtggttgctgccacccctagtctggctggccaatggactactagtgttggcctgcttggctcttctctttgtctacggggaacctgtgaccaggccacactccggcccggctgtacacttctggagacatcgcaaacaagctgacctggatttggcccggggagattttgcccaggccgctcaacagctgtggctggccttgcaagccctgggccggcccctgcccacctcaaacctggatctggcctgcagcctgctttggaacctcgtccgccacctgctgcagcgtctttgggtgggccgctggctggcaggccaggctgggggcctgcagagggactacaggctgagaaaggatgctcgtgccagtgcccgagatgcggctgtcgtctaccataagctgcaccagctgcatgccatgggcaagtacacaggaggccatcttgttgcttctaacctggcactgagtgcccttaacctggctgagtgtgcaggagatgctatatccatggcaacactggcagagatctacgtggcagctgccctaagggtcaaaaccagcctccccagagccttgcacttcttgacacgtttcttcctgagtagtgcccgccaggcctgcctggcacagagtggtgcagtgcctcttgccatgcagtggctctgccaccctgtaggtcaccgtttcttcgtggatggggactgggctgtacacggtgccccccaggagagtctgtacagcatggctgggaacccagtggatccactggcccaggtgacccgactattctgtgaacatctcctggagcgagcattgaactgtatcgctcagcccagcccaggggcagctgatggagacagggagttctcagatgctcttggatatctacagttgctaaatagctgttctgacgctgtcggagctcctgcgtgcagcttctctgtcagttccagcatggctaccaccactggcacagacccagtggccaagtggtgggcctcactgacagccgtggtgatccactggctgaggcgggatgaggaggcagctgaacgcttatacccactggtagagcacattccccaagtgctgcaggaaactgagagaccccttcccagggcagctctgtactccttcaaggctgcccgggctctgctggaccacagaaaggtggaatccagcccagccagcctggccatctgtgagaaggccagtgggtacctgcgggacagcttagcctctacatcaactgccagttccattgacaaggccatgcagctgctcctgtgtgatctacttcttgtggcccgcaccagcctatggcggcgccaacagtcagcagcttcagcccagggagctcacggtaccagcaatggaccccaggcctctgctctggagctgcgtggtttccaacatgacctgagcagcctgaggcgcttggcaca

SEQ ID NO: 8 - DNA, 1696 bp, rattus norvegicus
gcacgaggatcccagcgcggctcctggaggccgccaggcagccgcccagccgggcattcaggagcaggtaccatggattccttcaaggtagtgctggagggacctgccccttggggcttccgtctgcaagggggcaaggacttcaacgtgcccctctccatctctcggctcactcctggaggcaaggccgcacaggccggtgtggccgtgggagactgggtactgagtatcgacggtgagaacgccggaagcctcacacacattgaagcccagaacaagatccgtgcctgtggggagcgcctcagcctgggtcttagcagagcccagcctgctcagagcaaaccacagaaggccctgacccctcccgccgaccccccgaggtacacttttgcaccaagcgcctccctcaacaagacggcccggcccttcggggcacccccacctactgacagcgccctgtcgcagaatggacagctgctcagacagctggtccctgatgccagcaagcagcggctgatggagaatactgaagactggcgcccgcggccagggacaggccagtcccgttccttccgcatccttgctcacctcacgggcacagagttcatgcaagacccggatgaggaattcatgaagaagtcaagccaggtgcccaggacagaagccccagccccagcctcaaccataccccaggaatcctggcctggccccaccacccccagccccaccagccgcccaccctgggccgtagatcctgcatttgctgagcgctatgccccagacaaaaccagcacagtgctgacccgacacagccagccagccacacctacgcctctgcagaaccgcacctccatagttcaggctgcagctggagggggcacaggaggaggcagcaacaatggcaagacgcctgtatgccaccagtgccacaagatcatccgcggccgatacctggtagcactgggccacgcgtaccatcctgaggaatttgtgtgcagccagtgtgggaaggtcctggaagagggtggcttcttcgaggagaagggagctatcttttgcccctcctgctatgatgtgcgctatgcacccagctgtgccaaatgcaagaagaagatcactggagagatcatgcatgcgctgaagatgacctggcatgttccctgcttcacctgtgcagcctgcaaaacccctatccgcaacagggctttctacatggaggagggggctccctactgcgagcgagattacgagaagatgtttggcacaaagtgtcgcggctgtgacttcaagatcgatgccggggaccgtttcctggaagccctgggtttcagctggcatgatacgtgttttgtttgcgcaatatgtcaaatcaacttggaaggaaagaccttctactccaagaaggacaagcccctgtgcaagagccatgccttttcccacgtatgagcacctcctcacactactgccaccctactctgccagaagggtgataaaatgagagagctctctctccctcgacctttctgggtggggctggcagccattgtcctagccttggctcctggccagatcctggggctccctcctcacagtcccctttcccacacttcctccaccaccaccaccgtcactcacaggtgctagcctcctagccccagttcactctggtgtcacaataaacctgtatgtagctgtg

SEQ ID NO: 9 - DNA, 1290 bp, rattus norvegicus
cgcagccacctcagccccctgcggcggctccctcccggcctttcctcctacgagcagcatgaaagccttcagtccggtgaggtccgttaggaaaaacagcctgtcggaccacagcttgggcatctcccggagcaaaaccccggtggacgacccgatgagtctgctctacaacatgaacgactgctactccaagctcaaggaactggtgcccagcatcccccagaacaagaaggtgaccaagatggaaatcctgcagcacgtcatcgattatatcttggacctgcagatcgccctggactcgcaccccactatcgtcagcctgcaccaccagagacctggacagaaccaaacgtccaggacgccgctgaccaccctgaacacggacatcagcatcctgtccttgcaggcgtctgaattcccttctgagcttatgtcgaatgacagcaaagtactctgtggctaaataaatggcatttggggactttttcctttttttttttttttttttatttttactttctctttttcttttgcacaacaagaagtctacaggatcttttaagactgttgttttgttctgctttgttatcaaccatttcaccaggagaacaagttgaatggacctttttaaaaagaaaacggaaggaaaactaagagtgatcatctcccccaggtgttctccggcctggactgtgataaccgttatttatgagagactttcaatgccctttctacagtcggaaggttttctttatatacttattcccaccatggggagcgaaaatgttaaaaaaaaaaaaaaatcacaaggaattgcccaatctaagcagactttgccttttttcaaaggtggagcgtgaataccagaaggacccagtattcggttacttaaatgaagtctttggtcagaaatggcctttttgacacgagcctactgaatgctgtgtatatatttatatataaatatatatatatatattgagtgaaaccttgtggactctttaattagagttttcttgtacagtggcagagataactttctgcattgaaatgtaatgacgtacttatgctaaactttttataaaagtttggttgtaaacttaacccttttatacaaaataaatcaagtgtgtttgtcgattgctcgctttatttcagacaaccagtgctttgatttttttttatgctatgttataactgaacccaaataaataccagttcaaatttatgtagactgtattaagattataataaaatgtgtctgacatcaaaaaaaaaaaaaaaaaaaaa

SEQ ID NO: 10 - DNA, 3010 bp, rattus norvegicus
gagcgagcgggcgcaccaaggcgaggccagggccgaggcgcggggacggcggccgagagctaagcgaagcgctgggacggtgactgggaacggatcggagcgcgggacccggcgccgggtctcgtgcgtccccgccgatgggcgcgccgccgagccggtgctaactgggtcttccctgaagcagaaggaacagtatttcctacctttccgaaccgaagaccaagaagctagagaatctatgtaaataaactgaaatctctgttggctttgcacccccagcccggagccgtcacttttcctggtctgaggagcgtgtagacctcggccagccgctatgaatgtgaccagcttgttttcattcacaagcccagctgtgaagagactccttgggtggaaacagggcgacgaagaagagaaatgggcggagaaggctgtggacgctttggtgaagaaactgaagaagaagaaaggggccatggaagagctggagaaggccctgagctgccccgggcagcccagtaactgtgtcaccattcctcgctccctggatggcaggttgcaggtgtcccaccggaagggactgcctcatgtcatttattgccgcgtgtggcgctggcccgacctccagagccatcatgaactaaagcctctggaatgctgcgagttcccatttggttccaagcagaaggaagtctgcatcaatccctaccactataagcgagtggagagccccgttctcccaccggtgctggttccgaggcacagcgagtacaaccctcagcacagccttctggctcagttccgaaacctgggacaaaatgagccccacatgccactgaatgccacgttccccgactccttccagcagccacacagccacccgtttccccagtcccccaacagcagctaccccaactctcctggcagcagcagcagcacctaccctcactccccgaccagctcagacccgggcagcccttttcagatgccagccgacacacccccgcctgcttacctgcctcctgaagaccccatggcccaggatggctctcagcctatggacacgaacatgacgaacatgacggcaccgacactgcccgcggagatcaatagaggagatgttcaagcagttgcttacgaggaaccaaaacactggtgctctattgtgtactatgagctcaacaaccgtgtgggcgaagcgttccatgcctcctccaccagcgtgttggtggatggtttcaccgatccttccaacaataagaaccgattctgccttgggctgctctccaatgttaaccggaactccactattgaaaataccaggcgacacatcgggaaaggagtccacctttattacgtcggaggagaggtgtatgcggagtgcctcagtgacagcagcatcttcgtgcagagtcggaactgcaactaccaccatggctttcatcccaccacggtctgcaagatccccagcgggtgcagcttgaaaatcttcaacaaccaagagttcgctcagctactggcgcagtctgtgaaccatgggttcgagacagtgtatgaactcaccaaaatgtgcactattcgaatgagcttcgtgaagggctggggagcggagtaccaccggcaggatgtcactagcaccccctgctggattgagatacacccgcatggccctctccagtggctggataaagtccttacccagatgggttcgccccacaaccctatttcgtcggtgtcttaaagggccctcggcttctgtctcttgcaaactattgggccttgcatgtacttgaaggacggagaagtcagacaggatggggagctgtaaaggagccgtgatacttgacctctgtgaccaaccgttggatggagaagctgacaggccttggtaacaagctgttgatatcaagaacccgcttagtttacattgtgacattctgttgtaaaatcaactaaaatgctgacttttagcaggactttgtgaatagttagaaaagagatggccaagccaggggcaaattatctattaggaaaaaagcaaaaaaaaaggccctaagcaatccttttgtgtggggtgttggcagaaggttggcgccgatcatctttatggagtgggctctcatcaggctcggagcccacgctgatcatcctctcatgggttttcttaatattttaaaactatttgtttagaaatgaatgggtttttttgtttgttttaaagtaaaggttaattgttatgccatgcgtagtaacctttctgaagctgtatgctgactgtattgctgtcaggatgacgtcaggcatatgccctttgctaaatatgtatatacagaatacttggaggttatgaatagtccaaatggctagtgggtctacacagtatctgaggggcggggtcggggaggaaaaacgacagctgcaaatgtagacattgccgtaaagcccagcctgttgccttaaacggacaagctggtgtcggactttgctgtgtttcaggttttttagagttttacctggcttttcttcctttcttttctgctgtctcatccgctccacaatgcagttaagcagctggttaattcctctaactgtaagggcaaatgagtaattccttctgttcgcaaacggactggctctgcgtcccagttcccggaacatgaaaagcgtggatttaacgagcagttcttatggcgtttacaatacagacataggctttgatttttcaaataaattgtttgccaaacctggtaactctgttcattattcgcaggtttgaagaagatctctatcagaatccatttcaaagttttttttttttaattttgtttcatttttttttgtactatttggttttcttctgttgaagttttctattctccttttctcttatacagcgagttcttttattccaacactagcagggtttttctctactggaaatttttaaataaaacctgtcattattgcttaaaaaaaaaaaaaaaaaaaaaaaaaaaaaaaaaaaaaaaaa

SEQ ID NO: 11 - DNA, 4991 bp, rattus norvegicus
gaattcggcacgaggcgccggccagccgagagagtgagcgagcgcccgggacagaggagcgagcgggcgaggcgcgggcgaggctgggacccaagctcgctctctgctccgccaagtgccaacttcgcgccgactggctgggcgcgcacctttctgccgggcgatcagcgggaatttgagattttagggctgcgctctgtccccttcccctgagttcaaaatccctattaaaaagcaaaacaacagttacagcaaacttgctctcatcccgccggccccctcaactccggcaccatgaaggcggccgtcgatctcaagccgactctcaccatcatcaagacagaaaaagtggatttggagcttttcccatccccggatatggaatgcgcagatgtcccactgttaactccgagcagcaaagaaatgatgtcccaggcactgaaagctaccttcagtggtttcacaaaagagcagcagcgactgggaatccccaaagacccccggcagtggacagaaacccatgtccgggattgggtgatgtgggctgtgaatgagttcagcctgaagggtgtggacttccagaagttctgtatgaatggagcagcactgtgcgccctgggtaaagaatgcttcctcgagctggctccagactttgtgggggatatcctgtgggagcatctagagatcctgcagaaagaggatgtgaaaccgtatcaggttaatggagtcaaccctacctacccagaatcccgttacacctcggattacttcattagctatggtatcgagcatgctcagtgtgtccctccctcagagttctcagagcccagcttcatcacagagtcctatcagacgctgcatcccatcagctcggaagaactcctgtccctcaagtatgagaacgactacccttccgtcattctccgggaccctctccagacagacaccttgcagacagactactttgccatcaagcaagaagtgttaactccagacaacatgtgcatggggagagccagtcgtggtaaactcgggggccaggactcttttgagagcatagagagctacgatagttgtgaccgtctcacccagtcctggagcagccagtcatctttcaacagcctgcagcgtgtcccctcctatgacagcttcgactccgaggactatcccgctgccctgcccaaccacaagcccaagggcaccttcaaggactatgtgcgtgaccgtgctgacctcaacaaggacaagcctgtcattcctgctgctgccctggctggctacacaggaagtgggccgatccagctgtggcagtttcttctggaattactcactgataagtcttgtcagtcttttatcagctggacaggagacggctgggaattcaagctttccgacccagatgaggtggccaggagatggggaaagaggaaaaacaagcctaagatgaattatgagaaactgagccgcggccttcgctactattacgacaaaaatatcatccacaagacggcgggcaagcgctacgtgtaccgctttgtctgcgacctacagagcctgctgggatacaccccagaggagctgcatgccatgctggatgtgaagccggatgctgatgagtaacggacacggaaggggctgggggggaccgctgagacctttcaaagaacaacccgtgttggttggactcttaatttttaattgttattctatgttttattttccagaactcatttttcacattcaggggtgggagctaagggagttgcagccacattccattggccattggcaaggccggaaagggaaagtcaggacctgtggggtgggtggggcaagaagttcctgaggagatttttcaggagcgagagggtcttctcagaagccgagcgacttgacttacagaggaaaaggtttatgtgtccagtattctgtttgcaatcaaaggaaagagttcatcttgagttgtggatcgactagcaggaggaattgtcacgttaagatgaaagtcagtcagcgttggtgggtgggaggaaaacagtttcttcaggggatgactaaacgcaagaatttattaacctttctactttttttgaaacaaagatggactttaatggaagggatccaaaactgtttttattgttgaagtttattttattaaattctgtgccagtattttttttcttaaaaaaaatcgtcttaagctctaaggtggtctcagtattgcagtattgtgagttcgttgtaatttgctggctgaggagtctcccacaatgaaaggcagctgtttatatagaccccatggaaaaatccaagtctgtactgagaacagagacccccgaactcttacagctaaaggacgtgaatgctgatttggggaccagggaactgtgtgaattccacctgcggtttgtaaaacaacatttccagtccttttacttactgtcaggctgcgggcttagtgttagttaaggggcattaagtctttgcactgaatgtatttttgcagctctgctttggaactgttgtcagcataggtgcactgtttaagtcttggaaaagaaatcgaaggaggaagttgaacctggtttttggttaaatcgctacccgaaacatagaagactcaaactaaatgctgtttgcatgggcattacccctcggatcttaaggcctaaatgcatgccgttccaaactaacattgtacaatttctcctttatggcttatttccccagttcaccacattgctgcccctccccaccttactaatcagtagagtggaatgtaatgtttcctgataggtgaatgaaagtgtaatttcagacatcacagcacaaatcaaagagtacagactttctagggttcagagagcatggaaacgaggaggctatttcaggagcatgggacatggcctcctttgggaagaggctttcctttgaagaattggagcttgtcaggtaggatttcaggtgaggctgtttgcagatgaagtggtagagagcactgggacttcgagcttgacctgttgctacctcggcagagttaagcgaaagtcagctgcggatgagaggaaaacttgatggagggaatacaacggcaggagctaagaacggagaataaggtggcccgtgacagtgaggtatagttttactttcctcaaagtttaaattcaaagacttaatcgaggcagccatcttcctgccagttgagagaggagactggaaagtaaggcattgtgggtaaaaacaaagtaaggaaacatgcctggtatcattagaatccctcccactgtttaccacgcctagttgagacagcgactgtggacttccatgagagaaaagaggcagttggttgaggtggacctctccaggctcacccttcggggattgtaactacctggtatcatcttcatgagagttgatggctgactcccagattcccttgaagactctgaattcctgcgaacataacttgtccatgggtgttctgtactgaccgattggagcatccccaccggaagcaaaggcaaaacatcccagctctatgttttgatcttaacaaatgcaggtgccttaatgaagctctcaaagtatttaggagctgctcagggagtgttaggtgggatcatttggattatgttgtttttctcttatattatgtgatctttgttgggcactggcagtgtgtgtgtgtgtgtgtgggtgtgtgtgtgtacatttgtataagcctgcagctgaaatagtactgacttttctacttaagtctgtccacatttccataacggcgaaagagtacatacagccagggctgatttcatttctcgctctttgcaacagggcttaaataaaaaaaaaatcacttccccaagtcgtcgtccttgcccctattcagttcatgctgaaaacgcccctctcgagagcctaccgcctgcagccagtagcagataagacgtaagaaagtgcctcccacacggctcagcttgctgacttgcttctccccaggtccaccactttcaggatttgtagataggatattagtcagcttggtggtccatctggccagacgctctttcccatgctgtccaaaggccagagaccatcccaggaagagtggcgggtggtttatacactggaaatgtagcggcattgctgcattgatgctctacaaaaacacgttcacttcagaggaaggatgagcacatctgatccagctgggtggctccattgttttcccagagagatgctttaacctgtgcggttggcttttggctcagagctcaggggaataaggatgctccctgcatagagctctgaccgttgcgccatggaaaccagtggaagcactctagatcagagacactcttcctcactttggagatcaacactctgggttttaaagcattaaccttcatggtgaaatcacaccttctctcttctagccatgctgtgcatgccgctttctctgtcggggggtctctataaatttgttgaactcttacgtacattccaaagaagtttcaaggaaccacaagtatatgtatacaaatacatatatgaagtatatatgttaaaatgaaattatctctatcaggaatactgcctcagttaattgaattttttttaaggatacttttttttaagcggagaattattggggtgtaaaagatgttatattgtgtttgactattttccagcttgtattttcacataatttatattttttaaatgctgaaaatttaaaagcgagatttaaaaaggaaaagcaggtgctttttttttaaaaaaaagaatcagaactgaggtagcttagagatgtagccatgtaagtgtcttaaatgttttgtttttaaaaaaaaaacaaatgcaaaaaaattcttatgggggagttttttgtttgtttcttttagtagctgatgctggcacattttgctggaaagttttttatatactgtagcctgatttcatattgtattttaaactgtgctcgtgccgaattc

SEQ ID NO: 12 - DNA, 3251 bp, rattus norvegicus
atggccacccaggtgatggggcagtcttctggaggaggaagtctctttaacaacagtggcaacatgggcatggccttacccaacgacatgtatgacttgcacgacctctcgaaagctgaactggcggcacctcagctcattatgttagccaacgtggccctgactggggaagtgaatggcagctgctgtgattacctggttggtgaagagagacagatggccgagttgatgcctgttggagacaaccacttttcagatagcgaaggagaaggccttgaggagtcggctgaactaaaaggtgaccccagtgggctggacaacatggaactgagaagtttggagctaagcgttgtagagccccagcccgtatttgaagcatcagctgccccagaagtgtacagctcgaataaagatcccgcccctgaagcacccgtggcggaggacaaatgcaagaatttgaaggccaaacccttccgttgtaagccatgccagtatgaagcggagtctgaagaacagttcgtacatcacatccgggttcacagtgctaagaagttttttgtggaagagagtgcagagaagcaagccaaagccagggaatctggggcttccccgtctgaggagggcgagttctccaagggtcccatccgctgtgatcgctgtggctacaataccaaccggtatgatcactacacggcacacctgaagcaccacctgagagccggggataacgagcgtgtctacaagtgtatcatttgcacgtacacgacagtcagcgaataccactggcggaaacacctgaggaaccattttcccaggaaagtctacacgtgtagcaagtgcaactatttttcgacagaaaaaaataattatgttcaacacgttcgaactcacacaggagaacgcccttataaatgtgaactgtgtccttactcaagttctcagaagactcatctaactcgacacatgcgtactcactcaggtgagaagccatttaaatgtgatcagtgcaattatgtggcctctaatcagcacgaagtgacccgacacgcaagacaggttcacaacgggcctaaacctcttaattgccctcactgtgactacaaaacagccgataggagcaacttcaagaagcacgtcgagctgcacgtcaaccctcggcagttcaactgccccgtgtgtgactacgcggcctccaagaagtgtaacctgcagtaccatttcaagtccaagcaccccacctgccccagcaagacgatggacgtgtccaaagtgaagctgaagaaaaccaagaggagggaggctgacctgcaccgtgacgccgccgccgccgccactgagcagacggacacagagcaagcgaaaaccaagggggtggacgcgtctgcgaggagaagtgagaggcctgtaaaaggcgttggaaaagatgttccaaaagagaagaagccctgtagcaatgcctctgtggtgcaggtaactacccgaactcggaaatcagcggtggagactaaagcagcggagggaaaacacacagatggacagacaggaaacaacgcagaaaagtcctctaaagctaagaagagcaaaaggaagatggacgccgaggcccatccctcggtcgagcctgtgactgagggacccgtgacaaagaagaaaaagacggagagcaaacccaagaccagcggcgaagtgccgaagggcagcagagtggaggacaggaaggcggacaaacagcaaagtgcttccattaagaaaggcgggaagaagacggctctcaagactaagacagctaaaaaaggcagcaaacttgctccgaagtgggtggggcacacagaaccttcctcggagatggctcaaggaggggagtctccagttcctgctctcactcaggcggtggtcaccccatcaggatctactcagacagagctctcttctcccatggatattgctcagacagagcctgcccagatggacgtttcccagacagggccgcctcaggtgcagcggcctcttcctgtggagcctgctcaattggagccgtctcctcctcaggagcctccccaggtagagccacctgcctgtgtggagcctccccctcccgtggagcctccatgtcccatggagcctgctgagatggaaccgtcccctcccatggagccttcccaggtggagccacctcctcatttggagcctccgcttcccatggagctgcctcaggtggagctgcctcctgtggaggattgtcagaaggagctgcctcctgtggagcatgctcagactaaggttgctcagacaggtcctactcaggtgggagctgttcaggaggagccccttttctgtctccgagccacctcaagtcaagctaaccagaaggtcatctccccgaaagaccgtgccaaggagaagttgagcgtgctgagtgagatggcgaggcaggagcaggttcttattgaggttggcttagtgcctgtcagagatagccagcttctgaaggccagcaagagcgcaccggacctcccagccccaccgtcaccactgccaaagggacacttgagaagagaagagacacccaaggaccaagaaatgttctctgacggggaaggaaataaagtatcccctctcgagaaaggaggaacagaggaagctggtgagagtcgagctgagctggctgctcccatggaatctaccagtgctttatcctctgaacaaagctcaaatgcaccagatggtgaaacattacacagcgagtgtcaggctgactccactgcggtttgtgaaatggaagtggacactgagcagaagacagaccgtgtccctctgaaagactcagcagtagaaccagtgtcacctcttaacccaagagtggaccctgaagcagcggcaccagctgtggtggcctcccctcctatcactttggccgagtctcaggaaattgatgaggatgaaggcattcacagccatgatggaagtgacctgagcgacaacatgtctgaggggagtgatgactcaggactgcatggggctcggccagcaccacaggaagctacgtcaaaaagtggaaaggaagggttggctgtcaaagtaactgagggagagtttgtttgtattttttgtgatcgttcttttagaaaggaaaaagactatagcaaacacctcaatcgccatttggttaatgtgtacttccttgaagaagcagctgaggagcaggagtagagtagctgatcctcgaggagaagcgcaatgcgactttgtaa

SEQ ID NO: 13 - DNA, 996 bp, rattus norvegicus
gggggcacttccttcggacgcaagttattagcttactgcttctcggcctcgccaggaaactagtaacttcctctgggatcaaatagaattttataagaacacaatggatggagaggagaaaacctatggtggctgtgaaggccctgatgccatgtatgtgaaattaatatcttctgatggtcatgaatttattgtaaaaagagaacatgcattaacatcaggaacaataaaggccatgttgagtggtccaggtcagtttgcggagaatgaaaccaatgaggtcaactttagagagatcccttcacatgtgctatcgaaagtgtgcatgtattttacctacaaggtccgctatactaacagctccactgaaattcctgaattcccaattgcacctgaaattgcactggaactgctgatggccgcgaacttcctagattgttaaataaaataaattataataaactgttaatttttttcagtatttaatacctgtagttcagttagtaatttttcacatgtagcatgttgcctgtatgaagctgaactctaaagtgaatcgctgagcagatttcttctgtcatttgcatagcagagttgaaatttgtttgctacatcaacaaattaaggaagtttcacaagccaaaaaaataaataaataatgccagtttataggtggtttgctttctcctttggatgtgacctttaaagttatacctgatatagcaaatcctggaatgtaagcgtaaatgaatatattctacaggtaaatacaggggctaaggatttttatgtttttagtgtttttttaagaaccagttctgatcttaagccactgagcattattagaattatgcaaataattacatgaaaaacatgtttataacttagccgaaccttgatttttgtaactcaaagaataagagttgaaatttcttatgtgttttttgataaactgcagtattgtctaaaaaaaaaaaaaaaaaaaaaaaaaaaaaa

SEQ ID NO: 14 - DNA, 592 bp, rattus norvegicus
cctggctctgctcgctccgctccgctcgccaatcgcctccagagctccaggcatggccccaagtaccgtggcggtggagatgctcagtcccaaggagaaaaatcgactgcggaagccggtggtggagaagatgcgtcgggaccgcatcaacagcagcattgagcagctgaaactgctgctggagcaggagttcgcgcggcaccagcccaactccaaactggagaaggccgacatcctggagatggccgtcagctacctgaagcacagcaaagccttcgccgcggccgccggccccaagagcctgcaccaggactacagcgagggttactcgtggtgcctgcaggaggcggtgcagttcctgaccctgcatgcagccagcgacacgcagatgaagctgctttaccacttccagcggcccccagctcccgccgcgcctgtcaaggagaccccgacgcccggcgctgcgccccagccggcgcgctcctccactaaggccgccgcctccgtctccacctcgcgccaatctgcctgcggcctctggcggccctggtgacccagcggccgacgggtgcctggagctgaccagaggacc

SEQ ID NO: 15 - DNA, 1893 bp, rattus norvegicus
tgtctcactcagcgagacgcgcggtgcacggtgcttccccggcggacgaccgaccaacccgcgctccggcagagtccttggcgctcgcctgccggcgggacagacagcccgcctctagccgctctctggaccctggccgccccgagcgaagactggagcaaaatgatgcttcaacatccaggccaggtctctgcctcagaagtcagcgcgaccgccatcgtcccctgcctctcacctcctgggtcactggtgtttgaggattttgctaacctgacaccttttgtcaaggaagagctgagattcgccatccagaacaagcacctttgccatcggatgtcctctgcgctggagtcagtcaccatcaacaacagacctctggagatgtcagtcaccaagtctgaggtggcccctgaagaagatgagagaaaaaggaggcggcgggaaagaaacaaaattgctgctgccaagtgtcgaaacaagaaaaaagagaagacagagtgcctgcagaaggagtcagagaaactggagagtgtgaatgccgaactgaaggcccagatcgaggagctgaagaatgagaagcagcatctgatttacatgctcaacctgcaccggcccacgtgtatcgtccgggctcagaacgggcggacgccggaagacgagaggaacctttttatccaacagataaaagaaggaacattgcagagctaagcagaggtggcatgggggcaattggggagtccttactgaatcctccttttccaccccaaaccctgaagccattggaaaactggcttcctgtgcacttctagaatctcagcagccacgagctgttgggtcaggagggcctgcggtgactactgcgttgtcccactctgtccccgagtgaaccgtggagcaggcaggagcatcctttgtctcaccggctccaggatttaggccttaccatcccggccattctcagatgacctagctggccccaggctggggtcccatgcaaagcaggatcgcactaatgggatgcaggcagaagtgtctaccttgacaggtggggtggaccacgtcctccactgcggctgacaacatccctcctagggaagatggagtgagaacattcatcattgaagttgtccaatggccagggtatgctttctagaaactatgctgttctgtcctagactgactgtgcatagggcattcatttctgagcctggtgttgtgctatttagatgtttgtcttgcacaacattggcgtgatttttttccgggagtttcatcagacctgatttccgagagtttgggggtctgccactgtggacaatatcccccaaaagtgtttgggtggccatgtaaactggctgatgaccagctgtgctactctgtgctgaccgaggactgatgcctccttcccctgtacccactgctgaggaagaacccgggcacagcagctgtccttggctacaaactgttacaatgtcacagaacgaaggcacaaagtcccgctttcaaagggcgtaggactccacactcagtgacagggcaggaagagccaaggattctccgttttcccttccttcccaccaaaaaccacagcccgtggagactggtatttgaagccaggagtggggcaaggaaggtgtctgcactgtgggatgttaactgcgcttttgtcttgaagctattttgagatgcggtccagagtatttcagctgggaggtccctcccactggccaccagggctctggctactgttaaaattctgatgtttctgtgaaatcctcagtgttcaatccagactcagtagtatattacagttttctgtaagagagaacgttacttatttatcccagtattcctagcctgtcaacgtaataaaatatcagaatgagacctggt

SEQ ID NO: 16 - DNA, 4758 bp, rattus norvegicus
tggcgggtggaggatcgagcgctgttctcgctctggagccgctgcacatctcggaatcatttgcagcttattcactgtgtaaggaaaaaccaaattacatccataattctattgaactggaagcatagacgctgccatatatgtcacagtatacagaaaaagaaccatcagtaatggatcaagactccagcaaggctgcgtggcctagagcagcaggaggataccagacgattacaggcaggaggtacggaaggagacatgcgtatgtcagttttaagccatgtatgaccaggcatgaacggagcttgggtcgggctggcgatgactatgaagttctggaactggatgacgttgccaaggaaaacaccgcaggttccagttcattggatcaagtccatccttctttacccagtgaaactacagttgaaaaaagtgaaacagaaattcctacttgtggtccagcactgaatcaaagcacggagagcaacccatccgttgccacagtgtgtcatagtgaggaagtcagggagaccttagacagcagtacgaatcttcagaatcacgctgagagagagtgtacgccagcagtttgtaatgcctcaagtgtccagaatggaattgtgttggttcatactgactcttatgatccagacagcaagcatgatgagaatgactctcttcaactttgtgcccaagctgtggaaggtggtagacgtcagaaggtattaggcaatgcagtctttgagctggaaaatggagaggtagagagatatgctgatctgtgtccctcagttccctctctcagtggtgaaataagggaggagtctgaagagctaggttcagcactcttagagaaaaattctgctggcgatgcggaggctgtccatcaggatgggcaggaatttcagaggtcttctgaagacggcattgttagaaagaggcgacaagatgataccgatcagggaagacagacagaaaattcaactgaagatgcagactgtgttccaggccatgttgaacaaaatactagtgagagagccaatcaccatggaagttctcctgaacaggtagtgaggcccaaagttagaaaagtgatcagttcaagccaggtggaccaagagagcggttttaataggcacgaggctaagcaaagaagtgttcagaggtggagagaggctctggaagttgaggaatgcagttcagatgaccctataatcaagtgtgacgattatgatggagaccatgactgcatgttcctaaccccatcctactcaagagttacgccaagggaagcagaacgtcaccgtgcgacagcagaaaatggagccacagcttcaggaaggcaagaggctcgggaaaatgccttttggaatgcctgtggagagtattaccagctctttgacaaagacgaagacagttcagagtgcagtgatggggaatggtctgcttctctgcctcaccgattttctggcacagaaaaagaccagtcctcaagcgatgaaagctgggaaactctgccaggaaaagatgagaatgaacctgagctacagagtgatagtagtggccctgaggaagaaaaccaagaattgtctcttcaggagggagaacagacgtccttggaggagggggagattccctggttacagtacaatgaggtcaatgagagcagcagcgatgaagggaacgagcctgccaatgagtttgcacaaccagaagctttcatgttggatgggaacaacaacctggaggacgactcgagcgtgagtgaagacctggatgtggactggagcctatttgatggttttgcggatggacttggtgttgctgaagctatttcctacgtggatcctcagttcctcacctacatggcactagaagaacgcttagcccaggctatggagactgcactggcacacttagagtctcttgctgtagacgttgaggtggctaacccacctgccagtaaggaaagcatcgacggacttccagaaacccttgtcctagaagatcacaccgctattggtcaggagcagtgctgtcccatctgctgcagtgagtacattaaggatgacatcgcaacagagctgccctgccatcacttctttcataagccttgtgtctccatttggctacagaagtccggaacgtgccctgtgtgccgccgccacttcccacctgcagtgattgacgcatctgcagctgcttcctctgaaccagaccttgatgcctcgcctgcaaacgacaatgctgaggaagccccgtaagccgtcacggggaaatgagatcgaaattctatcacagtaaatctgcaaattccttctaaatctgatgtgcaaataattatatataaatatatttaaaatgctctatatagtatatgccgtagtttagaaagagaatattaacctttctaaactgaatttaggtttgcagaagatactaaacattttcaagctgaatgttgaagcagtgcatccattttctttagttgaacatggtgtccattgtaacgtcagtagtcatctctgtggcatacgtttctgttttgtctgaagtgctgccactaagagatcagaattaagctctcctttccacatcagatgtgaaaggagagcttaattctgatctctgtggcatacgtttctgttttgtctgaagtgctgccactaagagatcagaattaagctctcctttccacatcagatgtgaaaactgtgatcacaacagtagtacagtttggtttcattgaaaataaactgaattctaaagcatgctttttcactggtccctttgcttttgctacttcgagacctcttggtttatataacactgaggttaagattaaagcttttcagaatgccaggcaaagactagagttttgacccgaacacacacacaaaataaaaaaataaaaaataaattacatcttagatgtaaacttcctgaaactgtcatgtaataaactattcagtgaacgttggatgtttaaaggctagtaaagcatgtgaattatctgatgtgcttcatgctcggcacagtctcatgagcttggcctatgaaacactgtctaagccctcatgcagtctgcttctacttgttaaacactgaaggtccagaccagtcttggcatctgattaagaaattggatgtagttagctaaggtagtttgggttctactttaggagatctgaagatttgcacatggccatgtctgttacagcatcaccccagattagaaaggcacagcttttaggtggtcacacgagtaggcaagtctgtgcagacgctggtgtgataggtagcttacaggtgagccctgacatcagcacagggacaacggctcagcaggcccagcactggagcccatccgcgtgcacatctacctgccctgctcatccagttcgggaatatttctatagcttcccaaatcagcttcatctgatttgtgtaatatatctgagtatttatagtctttcttttttctccagtcttaaaaataaatgaattttcactgttggcacatttgaggcttaaatataagaaacctaacacttgcattctgatttttgcatatattgtaaatgtggctggtatttacagcgaatcctgtgtgtccttttatgggtaaaacaaaagtgaacattgcatgcaagtaatgtggtgaatttgtaatttaggggttttctggggcttctgtgactgtggatgcttaaattcaggccttaatgttgctctagctagcatgtttccttctaatgtacatagtcagcctgtatccactaatctctgcttgtcttctcctccgtggtcttcacaccgtatccactgatggtcagttagtgtcagaggtcacccaggttagcatcagttcccatgtgtgccgtgcggattgtgagccgtgtggttgcttggttggttttcttttatgtactttgattagctgtggcacttactgagtaaacttacgttgctgcagactgctgtgtaacaagccacgttttgtgtttagtcagtattacacacgggaccttgttttattcttctagatagcagctgtcccaaagaaaatatttcttctttgcctattaagatttagctattatctgccagttgttaaggggtttgattccaaactcaaccagagttgaacgtaagaaagctgctgtactcttgctgtccatctgttacgtcctccgttgttggctcctgactacgtacccacggctgctgaaggagacttgttttaatttgttttaaaaaggaaaatgaagttttaggactttgatggtggggaagctgacaagttcagaccccagtctgtcatttgctcttagcttggatccttttgaaaagttaagaatatatgaaggtaaattagaaaaagtaaaaatattgataaaatgtcatttatcttatgtctattttatgttgttatttggcctattaattttaattttttgaacattttcttatttcttgtaatatgaatgccgatatttaaagtttgactcacttgggtttcttttgtgtttcttagttgttatctctaagcctaactaacttttaagattatttgtgatatggatttatataagctgttaaatatatatgagctgttaaaatggaatgcaaggttttccaaagaccaggtctaactgtaatgattggtttattgttctacaatcccagcccggcattttccgtgtaaatcataaacaataaacaggatatactcagtgttcatttctaaaaaaaaaa

SEQ ID NO: 17 - DNA, 2855 bp, rattus norvegicus
gacaaaggggcaaagacagatgtcagaagggagccccttcctctgtcaggcgggcagtccacatgatgctttaactagatgcttatcactaaaggcgtggctgtcctgtttcggtgtctctctatgaccatctgccattctcttgtcctaaccctaaagtcaacccgggcaggtgacgacttctatgcttacgacgaggatggcacacgcttctcacctgacatcacgcccatcatcctggctgcgcattgccataaatacgaggtggtgcacctgctgctactcaagggtgcacgcatcgagcggccacacgactacttctgtcgctgcgccgactgtgctgagaagcaaaggcttgatgccttcagccactcaaggtctaggatcaatgcctacaagggactggccagcccagcatacttgtcgctgtccagcgaggaccctgtgctcacagcgctggaactcagcaatgagctggccaagctggccaacatagagaaggagttcaagaatgactacaggaagctctccatgcagtgcaaagacttcgtagtaggtgtgctggacctgtgccgggactcagaagaggtggaagccattctgaatggagatctggaatcggtggaacccctggagagacacgggcacaaggcgtcgctgagtcgggtcaaacttgccattaaatatgaagtcaaaaagtttgtggctcaccccaactgccaacagcagcttttgaccatctggtacgagaacctctcgggccttcgggagcagaccatctctatcaagtgtctggtcgtgttggtcgtggccttgggccttccattcctcgccatcggctactggattgcaccttgtaccaggctggggaaaattcttcgaagccccttcatgaagttcgtggctcacgctgcctccttcatcatcttcctgggtctgcttgtgttcaacgcctcacaccggtttgaaggcatcaccacgctgcccaacatcaccgttattgactaccccaagcaaatcttcagggtgaagaccacccagttcacatggacagaaatgctaattatggtctgggttctcgggatgatgtggtctgagtgcaaggagctgtggctggaggggccccgggagtacatcgtgcagctgtggaacgtgcttgacttcgggatgctctccatcttcattgctgccttcaccgccaggttcctagcatttctgcaagccaccaaagcgcaccagtatgtggacagccacgtgcaggagagcgacctgagcgaagtcacactcccaccgaaggttcagtatttcacctatgctagagataaatggcttccttctgaccctcagatcatatcggaaggcctctatgccatagctgtggtgctcagcttctcccggatcgcgtacattctccctgcaaacgagagctttgggcccttgcagatctctctggggaggactgtcgaagacatattccagttcatggttctcttcatcatggtgttcctggctttcatgattggcatgttcatactttactcctactaccttggggccaaagtaaaccctgcttttaccacggttgaagaaagtttcaagactttgttttggtccatatttggactctctgaagtgacttctgttgtgctcaaatatgaccacaaattcatagagaacattggctatgtcctttatggaatatacaatgtaactatggtggtcgttctgctcaacatgctgattgctatgattaacagctcataccaagaaatcgaggatgacagtgatgtagagtggaagtttgctcgttccaaactctggctatcctacttcgatgatggaaaaacattacctccacccttcagtctggtccctagtccaaaatcgtttgtttatttcatcatgaggatcactaacttttccaaatgcaggaggagaagacttcagaaggatctggaactgggcatgggtaactcaaagtccaggttaaacctcttcactcagtctaactcgagagtttttgaatcacacagttttaacagcattctcaatcagccaacacgatatcagcagataatgaaaagactcataaaacggtatgttttgaaagcacaagtagacaaagaaaatgatgaggtgaacgaaggtgaactgaaagaaatcaagcaggatatctccagccttcgttacgaacttttggaagataagagccaagcgacggaggaactggccatcttgattcataaactcagtgagaaactgaaccccagtgcgctgaggtgtgaatgagggagccacatgcagtgagactttgactctggcacaaatgtgggcaataatattcctaagtatgaaatacttgaaaaaccgttgtgtaaaaactctagtgttaataccttcaccatgcggagctttaaaattaacctagtgggccacaattcccattacgtgaaaatgatcccaaggttggcctcagcactccaagcagtgacatgctgttgtacttagaagcccagtattaatgtcactgatttttttttttttttgtcatttttgtctttatgtcacctgttggcctccaagagttccttgggggacagggaccacatcaatctcatctttatgtctccaacatttagtgcagtgcctgatacacagtaggtgcttggtaaatgttgaacccagctggcctggacagccagtagcatccaagaacacgaaagaccattgctatgtagcctaccttctcatccgaacgcttgaagcgatttttttaaaaatagaaactgaaggtgttttacgaccagccatgacgtgtggagatctttctcagacctttagacatgcactgacag

SEQ ID NO: 18 - DNA, 1238 bp, rattus norvegicus
cggggggaggcggcagcagtggagatggcggcggcggcggcggctccggggggcgggggcggggagcccaggggaactgctggggtcgtcccggtggtccccggggaggtggaggtggtgaaggggcagccattcgacgtgggcccacgctacacgcagctgcagtacatcggcgagggcgcgtacggcatggtcagctcagcatatgaccacgtgcgcaagaccagagtggctatcaagaagatcagccccttcgagcatcaaacctactgtcagcgcacgctgagagaaatccagatcttgctcggattccgccatgagaatgtcataggcatccgagacatcctcagagcacccaccctggaagccatgagagatgtttacattgttcaggacctcatggagacggacctgtacaagctgctaaagagccagcagctgagcaatgaccacatctgctacttcctctaccagatcctccggggcctcaagtacatacactcggccaatgtgctgcaccgggacctgaagccctccaatctgcttatcaacaccacctgcgaccttaagatctgtgattttggccttgcccggattgctgaccctgagcacgaccacactggctttctgaccgagtatgtggccacacgctggtaccgagccccagagatcatgcttaactccaagggctacaccaaatccattgacatctggtctgtgggctgcattctggctgagatgctctccaaccggcctatcttccccggcaagcactacctggaccagctcaaccacattctaggtatactgggttccccatcccaagaggacctaaattgtatcattaacatgaaggcccgaaactacctacagtctctgccctctaaaaccaaggtggcttgggccaagctttttcccaaatctgactccaaagctcttgacctgctggaccggatgttaacctttaacccaaacaagcgcatcacagtagaggaagcactggctcacccttacctggaacagtactatgatccgacagatgaaccagtggctgaggagccattcacctttgacatggagctggatgatctccccaaggagcggctgaaggagctgatcttccaagagacagcccgcttccagccaggggcaccagaggccccctaacaagaacagacacccctgtccttttggacctggtctgctctacctgctccttctctgcagattgttagaaa

SEQ ID NO: 19 - DNA, 1969 bp, rattus norvegicus
ctggcggctgtggacttgtgacatactaggtgacacccctggagccacttcctttcaactccagcttgaaagtgcctgcctggctcagggtctgcactgcagcctactcctagctccagggcttgactgcggcgtcagagcctatcctgtagcagcgcgccagcagccactcaaaccaaccacagctccccggcaacccgaatcatgaacaccgaaatgtatcagacgcccatggaggtggcggtctatcaactgcacaatttctccatctccttcttctcttctctgcttggaggggatgtggtttccgttaaactggataacagtgcctctggagccagcgtggtggccctagacaacaagattgagcaggccatggatctagtgaagaatcatttgatgtatgctgtgagagaggaggtggaggtcctaaaggagcagattcgtgagctggttgagaagaactcgcagctggagcgtgagaataccctcctgaagacgctggcgagcccagagcagctggagaagttccagtcccggctgagccccgaagagccagctcctgaagccccagaaaccccagaagcccctggcggttctgcggtgtaagtggctctgtccttagggtgggcagagccacaacttgttctacctagttctttccagtttgttttttggctccccaagcgtcatctcatgtggagaactttacacctaacatagctggtgccaagagatgtcccaaggacatgcccatctgggtccactccagtgacagacccctgacaaagagcaggtttggagactgagttgcacggggcctagtaaccccaagccagtgagcttgtcgtgccaccgggccctgggggctcccagggtctgggcaacttagttccagctggccaaggagaaagtagttttgagatggaatgccaatatgctccagaaagtataaggggtctgttttcatttccatggacatcttccacagcttcacctgacaacgactgttcctatgaagaagccacttgtgttttaagcagaagcaacctctctcttctcctctgtcttccagacggggacagagatgggagagattgagccaaatcagccttctgttggttaatattgtataatgcatggctttgtgcacagcccagtgtgggattacagctttgggatgactgcttataaagttctgtttggttagtattggcatagtttttctatatagccataaatgcgtatatatatatacccatagggctagaactatatattagtgtagtgatgtatacatatacacatacacatacatgttgaagggcctaaccagctttgggagtactgactggtctcttatctcttaaagctaagtttttgactgtgctaatttaccaaattgatccagtttgtcctttagattaaataagactcgagatataagggagggagagggagaccagcctcaccatgcggccacagatgccttgctgctgcagccctccccaatctgtccactgaagacatgaagtccttttgaatgccaaacccaccattcattggtgctgactacatagaatggggttgagagaagaccagtttggacttcacatttttgtttaagctttaggtcggttttttttttttttttgttttgttttcgtttgtttttgtttgttttttcttttcttttttttttaagttcttgtgggaaactttgggattagtcaaaggatggagtcctgtggtagacaagaagagtaaatagttttgatcccttcggggcctggaaaatgcacccaggaagcttgtgaaagaaggttctgtgacagtgaacactttccactttctgacaccttatcctgctgtatgtctccaggatttggattttgatttttcagatgtagcttgaaatttcaataaactttgctctttttttcaaaaaatcaaaaaaaaaaaaaaaaaa

SEQ ID NO: 20 - DNA, 479 bp, rattus norvegicus
  misc_feature (444)..(444): n is a, c, g, or t
ttttttttttttttttttggtgaaatttctactttatttatgatttggagaggaacatctgagatattacaatgcagtattcctcaattacaatgtggccatacaatcaaaagctctagaaagcagtcacaacaaaacaggaagcggatgtgctgttgagcggcatgactacagaaggctcttagcccaggagctcctttaacccccagttaaaactgccagcgcgctgcagaaggagctgccccaaagccacaccgggccctcatttgcggctttacttacttctttccgtttcgtttcaggacattccgactgcagagtaagagtcgcgccttcaatgtttcttggcatgggcgtggaaccagggtttatagtcaaatgaatctgatctggtgagataatgtgttgtacgtaaccctgggacattgcttcctgatctattangaagccctcttcaccttccaccagaaaaggcaagt

SEQ ID NO: 21 - DNA, 1990 bp, rattus norvegicus
cacgaatgtggtggaggctcagtgtggactgccccccgggaatgaagacaggcatcagatttcaaaattcagtcgaaagaacagcttcgtgggtcacagccatccgttcgttcctccgctggcaaccttggataacccataaaaggctcctgacaggttcaccctctgccggacagcagaaggaatattgatggattcgagaagctgaagcccttacaacctgaaagaagactggggaagtgattcgtctcctcctccatcgggtctgtaggacggggaggtcctttctgatgcagctgagaaaaatgcaggccatcaaaaaggagcctgcatccctggatcctaccggcagctcagacaagatgttgctgctgaactctgccttagctgaggtggctgaggacctagcctcaggggaagatctgctcctgaatgaagggagcatggggaaaaacaagtcctctgcctgtcggagaaaacgggaattcatcccggatgagaagaaggacgccatgtattgggagaaacggcggaaaaacaacgaggctgccaaaaggtctcgggagaagcgtcgcctcaatgacctggttctggagaacaagctgatcgccctgggagaagaaaatgccactttaaaagctgagctgctctccctgaaattaaagtttggcttaattagctccacggtgtacgcccaagaaatccagaaactcagtaattccacagctgtgtattttcaggactaccagacatccaaggctgccgtgagctcttacgtggacgagcatgagcctgcgatggtagccggaagttgcatctctgtcatcaagcactctcctcagagctcgctctcggatgtgtctgaggtgtcctctgtggagcacacacaggaaagccccgcacagggaggctgccggagccctgagaacaagttccctgtgatcaagcaggagcccgtggagttggagagctttgccagggagtccagagaggagcggggtgcctattccgcctccatctaccagagctacatgggtagctctttctccacctactcccactcccctcccctcctgcaggtccacggctccaccagcaactccccgagaacctcggaggccgacgagggtgtggtgggcaagtcttctgatggggaagacgagcagcaggtccccaaaggccccatccattctccagtggagctgcagcgggtgcacgccacggtggtgaaggttccggaagtgaacccttccgccttaccgcacaagctccggattaaagccaaggccatgcaggtcaaagtggaagctttggacagtgagttcgaaggcatgcagaaactctcttcaccggcagatgcgctagccaaaagacattttgacctggagaagcatggcgcttcgggtacggcccattcctccctccctcctttctcggtgcaggtgacgaacattcaagattggtccctcagatcggaacactggcatcacaaagaactgggcggcaaaactcagggtaccttcaaaacgggtgtggtggaagtcaaagacagtggctataaggtttccgaggctgagaatttgtatttgaagcagggggtggcaaacttatccgcagaggtggtctcgctcaagagattcatagccacacagcctatctcggcttcggactccaggtaaatggctactgactgagctatgcgtggaagaggatgctcttggtaccatactgaatttcccactggacctctaaagtcatttcactgtagtgtgcacaacggtgtctgtctggtgtccttgtgtacacaacgctgaagacttgatgccctcactctgcctggcgtgtagtcagatagcccttccctccccaccccccagaggctgtacataccgaacgttatttttgctctatgataaagtgtgtacgttgccagtttcaataaaggattggtggcaaacacaaaaaaaaaaaaaaaaaaaaaaaaaaaaaa

SEQ ID NO: 22 - DNA, 2882 bp, homo sapiens
gtccggccagggtggttggtggtaaggattcaggctccgtcctaacgaggccgtggcctgaggctcagggccccccgcccctccctcccagcccaccagcgtcacctcccagccccgagctggaccgcacaccttgggacacggttttccacttcctaaggacgagccccagactggaggagaggtccgaggaggtgggcgttggactctttgcgaggaccccggcggctggcccgggggaggcggccgaggcggcggcggcggcggccgggggcgacatggcggaggagcaggacctatcggaggtggagctgagccccgtgggctcggaggagccccgctgcctgtccccggggagcgcgccctcgctagggcccgacggcggcggcggcggatcgggcctgcgagccagcccggggccaggcgagctgggcaaggtcaagaaggagcagcaggacggcgaggcggacgatgacaagttccccgtgtgcatccgcgaggccgtcagccaggtgctcagcggctacgactggacgctggtgcccatgcccgtgcgcgtcaacggcgccagcaaaagcaagccgcacgtcaagcggcccatgaacgccttcatggtgtgggctcaggcagcgcgcaggaagctcgcggaccagtacccgcacctgcacaacgctgagctcagcaagacgctgggcaagctctggaggctgctgaacgaaagtgacaagcgccccttcatcgaggaggctgagcggctccgtatgcagcacaagaaagaccacccggactacaagtaccagcccaggcggcggaagaacgggaaggccgcccagggcgaggcggagtgccccggtggggaggccgagcaaggtgggaccgccgccatccaggcccactacaagagcgcccacttggaccaccggcacccaggagagggctcccccatgtcagatgggaaccccgagcacccctcaggccagagccatggcccacccacccctccaaccaccccgaagacagagctgcagtcgggcaaggcagacccgaagcgggacgggcgctccatgggggagggcgggaagcctcacatcgacttcggcaacgtggacattggtgagatcagccacgaggtaatgtccaacatggagacctttgatgtggctgagttggaccagtacctgccgcccaatgggcacccaggccatgtgagcagctactcagcagccggctatgggctgggcagtgccctggccgtggccagtggacactccgcctggatctccaagccaccaggcgtggctctgcccacggtctcaccacctggtgtggatgccaaagcccaggtgaagacagagaccgcggggccccaggggcccccacactacaccgaccagccatccacctcacagatcgcctacacctccctcagcctgccccactatggctcagccttcccctccatctcccgcccccagtttgactactctgaccatcagccctcaggaccctattatggccactcgggccaggcctctggcctctactcggccttctcctatatggggccctcgcagcggcccctctacacggccatctctgaccccagcccctcagggccccagtcccacagccccacacactgggagcagccagtatatacgacactgtcccggccctaaagggggccctgtcgccaccaccccccgcccagcccctgcccccagcctgtgtgccctgttccttgcccacctcaggcctggtggtggcagtggaggaggctgaggaggctgaagaggctgacaggtcggggggctttctgtctggctcactgccctgatgacccacccgccccatccaggctccagcagcaaagccccaggagaacaggctggacagaggagaaggaggttgactgttgcacccacactgaaagatgaggggctgcaccttcccccaggaatgaccctctatcccaggacctgagaagggcctgctcaccctcctcggggaggggaagcaccagggttggtggcatcggaggccttaccactcctatgactcctgttttctctctcacagatagtgagggtctgacatgcccatgccacctatgccacagtgcctaagggctaggccacccagagactgtgcccggagctggccgtgtctcccactcaggggctgagagtagctttgaggagcctcattggggagtggggggttcgagggacttagtggagttctcatcccttcaatgccccctccctttctgaaggcaggaaggagttggcacagaggccccctgatccaattctgtgccaataacctcattctttgtctgagaaacagcccccagtcctcctccactacaacctccatgaccttgagacgcatcccaggaggtgacgaggcaggggctccaggaaaggaatcagagacaattcacagagcctccctccctgggctccttgccagctccctcttcccttactaggctctatggcccctgctcagtcagccccactccctgggcttcccagagagtgacagctgctcaggccctaacccttggctccaggagacacagggcccagcacccaggttgctgtcggcaggctgaagacactagaatcctgacctgtacattctgcccttgcctcttaccccttgcctcccagtggtatttgaataaagtatgtagctatatctgcccctattttcctgttctgcagccccccaaatccacatgtaactcattactgtctcctgttatttatctcagtagtcccctctcctagccactctagcccctattaactctgcattaagcattccacataataaaattaaaggttccggttaaaaaaaaaaaaaaaaaaaaa

SEQ ID NO: 23 - DNA, 1661 bp, homo sapiens
cggccgctgggtccgtgaaggggagtgaggcactcctcaccctgtggcagctcctagtcaggaggcctctcccctccacctgcacgtcatgtggctgtggctcccaggaccagcgcctggagcgggtgagtgaacgtccctctcctccgtgggcactgcctccgctgtgagccacccttcaggctacgtgcatttccgcctgttctcagggtttaaccgaaaccgaacattaaccgtagccctaacgcggctgagggatccaggccttgcctctgggccgggtgagagacagacactcgccaaccagtccgcagtggctccccgtgtgtgcgaggctcccgcctgcgtgtgtgcgtgtgcatgtgtgcctgcgcacaggggagggaaagttcttcagctgccgagggcgggagtaggggctggagcagcaccctgcggcgaggccccacaggagaatcgatggttctgatgctgtcagcctctgggtgcaaattctgagggcccgggagcgtctggcggccctgagcagcttgctggggaagctccgtggaggaggaagtccggaggcatgtgatgtgaatcagctgacatcatcttgacgggagccctgtgaggcggacacttaactccagtttcacaggagggagccatggatttgctgcgggagctgaaggccatgcctatcacgctgcacctgctccagtccacccgagtcgggatgtctgtcaacgcccttcggaagcagagctcggatgaggaggtcattgcactggccaagtctctcatcaagtcctggaagaagctcctggatgcttccgatgccaaagccagggagcgggggaggggcatgcctctgcccacgtcctcgagggatgcctcagaggccccggatcccagccgcaagaggccggagctgcccagggcaccgtcgactccgaggatcaccacatttcctccggtgcctgtcacctgtgatgccgtgcgcaacaagtgccgcgagatgctgaccgctgccctgcagacggaccatgaccacgtggccatcggtgcggactgcgagcgcctgtcggctcagatcgaggaatgcatcttccgggacgttggaaacacagacatgaagtataagaaccgtgtacggagtcgtatctccaacctgaaggatgccaagaaccctgacctgcggcggaatgtgctgtgtggggccataacaccccagcagatcgctgtgatgacctcagaggagatggccagtgatgagctgaaggagatccgtaaggccatgaccaaggaggccatccgagagcaccagatggcccgcactggcggcacgcagacagacctgttcacctgcggcaagtgcaggaaaaagaactgcacctacacacaggtgcagacccgcagctctgatgagcccatgaccacctttgttgtctgcaacgagtgtggaaaccgctggaagttctgctgacccctcgtgtagatgtgctgcagccttgggccctccccggcccacgtcctccgttgacacagcttctctggagaccctagaaggcggcatgtcctgccctcaacctgcctgcctggattgcacctttctgccctttccccctcattattaaatgtttctttttgccaaaaaaaacaaaaaaaaaaaaaaaaaa

SEQ ID NO: 24 - DNA, 1353 bp, homo sapiens
cccttcccccagcaggccaaggctggtgacagccttcatatatttaaagaggacaagagcccctcagactcagttgagctgaacggagtccacacaggcagacttgaccttgaccagatagtcttcaagatgccaaactggggcggaggcgcaaaatgtggagcctgtgaaaagaccgtctaccatgcagaagaaatccagtgcaatggaaggagtttccacaagacgtgtttccactgcatggcctgcaggaaggctcttgacagcacgacagtcgcggctcatgagtcggagatctactgcaaggtgtgctatgggcgcagatatggccccaaagggatcgggtatggacaaggcgctggctgtctcagcacagacacgggcgagcatctcggcctgcagttccaacagtccccaaagccggcacgctcagttaccaccagcaacccttccaaattcactgcgaagtttggagagtccgagaagtgccctcgatgtggcaagtcagtctatgctgctgagaaggttatgggaggtggcaagccttggcacaagacctgtttccgctgtgccatctgtgggaagagtctggagtccacaaatgtcactgacaaagatggggaactttattgcaaagtttgctatgccaaaaattttggccccacgggtattgggtttggaggccttacacaacaagtggaaaagaaagaatgaagaggtgcgccgtttctcagattttttgcgagcctaaaacacttgccaagtaatcctgcacagatcgatacctttccccaaatagcctctcctttgtagtcgtacattatgtgtttctcctcagaagtgatcaggtctttactgaatgttagaagaggcctttggaagaaaattatgtaaagtttaatctataacaaatgctttattatttataatgcttggaatgggagaggcaataaataaatgttttagtgctatcttgtatggctctagatcttttctttgagatagaaattttcaaaaacataaagctagttcaaaaaacgagttgcagagcatataataaatttggatgtcaactgagaaaggagtgagaaggaagaaacaatgcgcaaaggaaagcagtctttcagaatctgtcagccaagtgtctttctagttactgctaatggagaagaaaacagggggtctgggagaaaatagagaacatgatagcaaaatctaaaaggaaaatcaaaactaataaaattgctgaagagttgatccctttgtcctatcgtggggctttgtaatgttacacatctcgtgaaaactcagaaatgacaataaagcgtggcatttgcctctgtattataaatg

SEQ ID NO: 25 - DNA, 1994 bp, homo sapiens
catattcagcagggttagcttgtcctcccctccctcttcagcttcccagacactgagtctggaatgaaaattcacctgcctctgagttggctcctaatgggggtgggagtgttacttcggttcccaggttggaagattatctcacccggccccagctatataagctgaccggtgtggaggggcccagcagggccaactccagggattccttccacgacagaaaaacatacaagactccttcagccaacatgatggtactgaaagtagaggaactggtcactggaaagaagaatggcaatggggaggcaggggaattccttcctgaggatttcagagatggagagtatgaagctgctgttactttagagaagcaggaggatctgaagacacttctagcccaccctgtgaccctgggggagcaacagtggaaaagcgagaaacaacgagaggcagagctcaaaaagaaaaaactagaacaaagatcaaagcttgaaaatttagaagaccttgaaataatcattcaactgaagaaaaggaaaaaatacaggaaaactaaagttccagttgtaaaggaaccagaacctgaaatcattacggaacctgtggatgtgcctacgtttctgaaggctgctctggagaataaactgccagtagtagaaaaattcttgtcagacaagaacaatccagatgtttgtgatgagtataaacggacagctcttcatagagcatgcttggaaggacatttggcaattgtggagaagttaatggaagctggagcccagatcgaattccgtgatatgcttgaatccacagccatccactgggcaagccgtggaggaaacctggatgttttaaaattgttgctgaataaaggagcaaaaattagcgcccgagataagttgctcagcacagcgctgcatgtggcggtgaggactggccactatgagtgcgcggagcatcttatcgcctgtgaggcagacctcaacgccaaagacagagaaggagataccccgttgcatgatgcggtgagactgaaccgctataagatgatccgactcctgattatgtatggcgcggatctcaacatcaagaactgtgctgggaagacgccgatggatctggtgctacactggcagaatggaaccaaagcaatattcgacagcctcagagagaactcctacaagacctctcgcatagctacattctgaggcaaacgacagactcttaatcagtaaatgttcactggcattttgaaggcatggcccaagagaagagacactagccataaaatctagtttctatttatcaacgtgttgtgaagatgtacctaatgaagttttgagaaagcacagggttataggtgtttaaatttcctttagtgaaactcttatttatttttatgtattcctgtttatttatttactgccacgctactgatattcagaccttcatgatcatccatctggtgagcagagcttcatttgtatataacactttcagagccttcccacccataggtagttcttaaaccaggtgaaagagcaaagttcaagtgcctacttatgtgtcattcgctcatgtaagagtttttaagagagggctgattatcacagccctcttttctcctgaatttttaatgcagaagtttgaatgaagcaagggaaggcatgtagggacaggaaaggaaacaatggaaggaaagtgattctgtgaaaaggacagtgaagccagctattttacccccaggctggattttttttttttttttttttttttttttttaccgagtacacagagtacccaagtgaagagaacgtcatgagtgtaagtgcaaatcagtggaaggagcggcaaactgggacatgcagaattgaatttgctcaaaaaagatgaaaggaaatgcaaactgtaaatgtataaatgtatattgtattgtatgtacattttatattcataataaaggcaatcaaactcaaaaaaaaaaaaaaaaaaaa

SEQ ID NO: 26 - DNA, 5288 bp, homo sapiens
ggaccggaagtggggggcggaagtgcagtgggctcagcgccgactgcgcgcctctgcccgcgaaaactctgagctggctgacagctggggacgggtggcggccctcgactggagtcggttgagttcctgagggaccccggttctggaaggttcgccgcggagacaagtgagcagtctgtgccatagggattctcgaagagaacagcgttgtgtcccagtgcacatgctcgcatcgcttaccaggagtgcccgagaccctaagatgttcggagtggttttttcgcacagacccgaatagcctgcccctcagccacgctctgtgcccttctgagaacaggctgatatgcccaagatagtcctgaatggtgtgaccgtagacttccctttccagccctacaaatgccaacaggagtacatgaccaaggtcctggaatgtctgcagcagaaggtgaatggcatcctggagagccctacgggtacagggaagacgctgtgcctgctgtgcaccacgctggcctggcgagaacacctccgagacggcatctctgcccgcaagattgccgagagggcgcaaggagagcttttcccggatcgggccttgtcatcctggggcaacgctgctgctgctgctggagaccccatagcttgctacacggacatcccaaagattatttacgcctccaggacccactcgcaactcacacaggtcatcaacgagcttcggaacacctcctaccggcctaaggtgtgtgtgctgggctcccgggagcagctgtgcatccatcctgaggtgaagaaacaagagagtaaccatctacagatccacttgtgccgtaagaaggtggcaagtcgctcctgtcatttctacaacaacgtagaagaaaaaagcctggagcaggagctggccagccccatcctggacattgaggacttggtcaagagcggaagcaagcacagggtgtgcccttactacctgtcccggaacctgaagcagcaagccgacatcatattcatgccgtacaattacttgttggatgccaagagccgcagagcacacaacattgacctgaaggggacagtcgtgatctttgacgaagctcacaacgtggagaagatgtgtgaagaatcggcatcctttgacctgactccccatgacctggcttcaggactggacgtcatagaccaggtgctggaggagcagaccaaggcagcgcagcagggtgagccccacccggagttcagcgcggactcccccagcccagggctgaacatggagctggaagacattgcaaagctgaagatgatcctgctgcgcctggagggggccatcgatgctgttgagctgcctggagacgacagcggtgtcaccaagccagggagctacatctttgagctgtttgctgaagcccagatcacgtttcagaccaagggctgcatcctggactcgctggaccagatcatccagcacctggcaggacgtgctggagtgttcaccaacacggccggactgcagaagctggcggacattatccagattgtgttcagtgtggacccctccgagggcagccctggttccccagcagggctgggggccttacagtcctataaggtgcacatccatcctgatgctggtcaccggaggacggctcagcggtctgatgcctggagcaccactgcagccagaaagcgagggaaggtgctgagctactggtgcttcagtcccggccacagcatgcacgagctggtccgccagggcgtccgctccctcatccttaccagcggcacgctggccccggtgtcctcctttgctctggagatgcagatccctttcccagtctgcctggagaacccacacatcatcgacaagcaccagatctgggtgggggtcgtccccagaggccccgatggagcccagttgagctccgcgtttgacagacggttttccgaggagtgcttatcctccctggggaaggctctgggcaacatcgcccgcgtggtgccctatgggctcctgatcttcttcccttcctatcctgtcatggagaagagcctggagttctggcgggcccgcgacttggccaggaagatggaggcgctgaagccgctgtttgtggagcccaggagcaaaggcagcttctccgagaccatcagtgcttactatgcaagggttgccgcccctgggtccaccggcgccaccttcctggcggtctgccggggcaaggccagcgaggggctggacttctcagacacgaatggccgtggtgtgattgtcacgggcctcccgtaccccccacgcatggacccccgggttgtcctcaagatgcagttcctggatgagatgaagggccagggtggggctgggggccagttcctctctgggcaggagtggtaccggcagcaggcgtccagggctgtgaaccaggccatcgggcgagtgatccggcaccgccaggactacggagctgtcttcctctgtgaccacaggttcgcctttgccgacgcaagagcccaactgccctcctgggtgcgtccccacgtcagggtgtatgacaactttggccatgtcatccgagacgtggcccagttcttccgtgttgccgagcgaactatgccagcgccggccccccgggctacagcacccagtgtgcgtggagaagatgctgtcagcgaggccaagtcgcctggccccttcttctccaccaggaaagctaagagtctggacctgcatgtccccagcctgaagcagaggtcctcagggtcaccagctgccggggaccccgagagtagcctgtgtgtggagtatgagcaggagccagttcctgcccggcagaggcccagggggctgctggccgccctggagcacagcgaacagcgggcggggagccctggcgaggagcaggcccacagctgctccaccctgtccctcctgtctgagaagaggccggcagaagaaccgcgaggagggaggaagaagatccggctggtcagccacccggaggagcccgtggctggtgcacagacggacagggccaagctcttcatggtggccgtgaagcaggagttgagccaagccaactttgccaccttcacccaggccctgcaggactacaagggttccgatgacttcgccgccctggccgcctgtctcggccccctctttgctgaggaccccaagaagcacaacctgctccaaggcttctaccagtttgtgcggccccaccataagcagcagtttgaggaggtctgtatccagctgacaggacgaggctgtggctatcggcctgagcacagcattccccgaaggcagcgggcacagccggtcctggaccccactggaagaacggcgccggatcccaagctgaccgtgtccacggctgcagcccagcagctggacccccaagagcacctgaaccagggcaggccccacctgtcgcccaggccacccccaacaggagaccctggcagccaaccacagtgggggtctggagtgcccagagcagggaagcagggccagcacgccgtgagcgcctacctggctgatgcccgcagggccctggggtccgcgggctgtagccaactcttggcagcgctgacagcctataagcaagacgacgacctcgacaaggtgctggctgtgttggccgccctgaccactgcaaagccagaggacttccccctgctgcacaggttcagcatgtttgtgcgtccacaccacaagcagcgcttctcacagacgtgcacagacctgaccggccggccctacccgggcatggagccaccgggaccccaggaggagaggcttgccgtgcctcctgtgcttacccacagggctccccaaccaggcccctcacggtccgagaagaccgggaagacccagagcaagatctcgtccttccttagacagaggccagcagggactgtgggggcgggcggtgaggatgcaggtcccagccagtcctcaggacctccccacgggcctgcagcatctgagtggggtgagcctcatgggagagacatcgctgggcagcaggccacgggagctccgggcgggcccctctcagcaggctgtgtgtgccagggctgtggggcagaggacgtggtgcccttccagtgccctgcctgtgacttccagcgctgccaagcctgctggcaacggcaccttcaggcctctaggatgtgcccagcctgccacaccgcctccaggaagcagagcgtcatgcaggtcttctggccagagccccacaaggaccatgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgtgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactgatcctggccccctcttatttattctacatccttggcaccccacttgcactgaaagaggcttttttttaaatagaagaaatgaggtttcttaaagcttatttttataaagctttttcataaaactggttgtagttgc

SEQ ID NO: 27 - DNA, 3641 bp, homo sapiens
ctgcactgaagagggagagcgagagagagactggagacgcacagatccccccaaggtctcccaagcctaccgtcccacagattattgtacagagccccaaaaatcgaaacagaggaaacgaacagcagttgaacatggacgaaggaattcctcatttgcaagagagacagttactggaacatagagattttataggactggactattcctctttgtatatgtgtaaacccaaaaggagcatgaaacgagacgacaccaaggatacctacaaattaccgcacagattaatagaaaagaaaagaagagaccgaattaatgaatgcattgctcagctgaaagatttactgcctgaacatctgaaattgacaactctgggacatctggagaaagctgtagtcttggaattaactttgaaacacttaaaagctttaaccgccttaaccgagcaacagcatcagaagataattgctttacagaatggggagcgatctctgaaatcgcccattcagtccgacttggatgcgttccactcgggatttcaaacatgcgccaaagaagtcttgcaatacctctcccggtttgagagctggacacccagggagccgcggtgtgtccagctgatcaaccacttgcacgccgtggccacccagttcttgcccaccccgcagctgttgactcaacaggtccctctgagcaaaggcaccggcgctccctcggccgccgggtccgcggccgccccctgcctggagcgcgcggggcagaagctggagcccctcgcctactgcgtgcccgtcatccagcggactcagcccagcgccgagctcgccgccgagaacgacacggacaccgacagcggctacggcggcgaagccgaggcccggccggaccgcgagaaaggcaaaggcgcgggggcgagccgcgtcaccatcaagcaggagcctcccggggaggactcgccggcgcccaagaggatgaagctggattcccgcggcggcggcagcggcggcggcccggggggcggcgcggcggcggcggcagccgcgcttctggggcccgaccctgccgccgcggccgcgctgctgagacccgacgccgccctgctcagctcgctggtggcgttcggcggaggcggaggcgcgcccttcccgcagcccgcggccgccgcggcccccttctgcctgcccttctgcttcctctcgccttctgcagctgccgcctacgtgcagcccttcctggacaagagcggcctggagaagtatctgtacccggcggcggctgccgccccgttcccgctgctataccccggcatccccgccccggcggcagccgcggcagccgccgccgccgctgccgccgccgccgccgcgttcccctgcctgtcctcggtgttgtcgccccctcccgagaaggcgggcgccgccgccgcgaccctcctgccgcacgaggtggcgccccttggggcgccgcacccccagcacccgcacggccgcacccacctgcccttcgccgggccccgcgagccggggaacccggagagctctgctcaggaagatccctcgcagccaggaaaggaagctccctgaatccttgcgtcccgaaggacggaggttcaagcagagtgagaagttaaaatacccttaaggaggttcaagcagagtgagaagttaaaatacccttaaggtctttaagggaggaagtgtaatagatgcacgacaggcataaacaagaacaacaaaacaggtgttatgtgtacattcggagttcctgttttgctcatcccgcaccaccccaccctccacacactaacatccctttcttccccccaccagctgtaaaagatcctatgcgaaagacactggctcttttttttaatcccccaaataaattttgcccccttttaggccatgttccattatctcttaaaattggaacctaattcgagaggaagtaagaagggtctgttctgtggctgagctaggtgaaccccggggtaggggaaagatgttaacacctttgacgtctttggagttgacatggaacagcaggtagttgttatgtagagctagttctcaaagctgccctgcctgttttaggaggcgttccacaaacagattgaggctctttttagaattgaatttactcttcagtattttctaatgttcagctttctaaaaggcatatatttttcaaagaagtgaggatgcagtttctcacgttgcaacctattctgaagtggtttaaatggtatctcttagtaacttgcactcgttaaagaaacacggagctgggccatcgtcagaactaagtcagggaaggagatggatgagaaggccagaatcattcctagtacatttgctaacactttattgagaaattgaccatgaattaatggactcatcttaatttcttctaagtccatatatagatagatatctatctgtacagatttctatttatccatagataggtatctatacatacacatctcaagtgcatctattcccactctcattaatccatcatgttcctaaatttttgtaatcttactgtaaaaaaaagtgcactgaacttcaaaacaaaacaaaaaacaacaacaacaaaaaacaagtccaaactgatatatcctatattctgttaaaattcaaaagtgaacgaaagcatttaactggccagttttgattgcaaatgctgtaaagatatagaatgaagtcctgtgaggccttcctatctccaagtctatgtattttctggagaccaaaccagataccagataatcacaaagaaagcttttttaataaggcttaaaccaagaccttgtctagatatttttagtttgttgccaaggtagcactgtgagaaatctcacttggatgttatgtaaggggtgagacacaacagtctgactatgagtgaggaaaatatctgggtcttttcgtcagtttggtgcatttgctgctgctgttgctactgtttgcctcaaacgctgtgtttaaacaacgttaaactcttagcctacaaggtggctcttatgtacatagttgttaatacatccaattaatgatgtctgacatgctatttttgtagggagaaaatatgtgctaatgatattttgagttaaaatatcttttggggaggatttgctgaaaagttgcacttttgttacaatgcttatgcttggtacaagcttatgctgtcttaaattattttaaaaaaattaaatactgtctgtgagaaaccagctggtttagaaaagtttagtatgtgacgataaactagaaattacctttatattctagtattttcagcactccataaattctattacctaaatattgccacactattttgtgatttaaaaattcttactaaggaataaaaactttaatatacgatatgatattgtctaataattaaaaaagacataatggatgctcaattagttttaagatatctataactatagggatacaaatcactacagttctcagatttacaccttttttttgtcattggcttgatgtcacacatttccaatctcttgcaagcctccaggctctggctttgtctacctgctcgttcccaatgtatcttaatgaaaagtgcaaaagaaaaacctaccaattaaaaaaaaaaaaaaaaaa

SEQ ID NO: 28 - DNA, 4284 bp, homo sapiens
agcagagctgcggccgggggaacccagtttccgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaaggtgaagtcggcgcggggaggggtagggccaacggcctggacgccccaagggcgggcgcagatcgcggagccatggattgcactttcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttataatctggaaaaaa

SEQ ID NO: 29 - DNA, 1726 bp, homo sapiens
agaacactggcggccgatcccaacgaggctccctggagcccgacgcagagcagcgccctggccgggccaagcaggagccggcatcatggattccttcaaagtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcagcagaatgggtgcagacccctgacaaacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgctgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctggggctccctgggccctgccccacccaccttatcctcccaccccactccctccaccaccacagcacaccggtgctggccacaccagccccctttcacctccagtgccacaataaacctgtacccagctgtg

SEQ ID NO: 30 - DNA, 1402 bp, homo sapiens
ggggacgaagggaagctccagcgtgtggccccggcgagtgcggataaaagccgccccgccgggctcgggcttcattctgagccgagcccggtgccaagcgcagctagctcagcaggcggcagcggcggcctgagcttcagggcagccagctccctcccggtctcgccttccctcgcggtcagcatgaaagccttcagtcccgtgaggtccgttaggaaaaacagcctgtcggaccacagcctgggcatctcccggagcaaaacccctgtggacgacccgatgagcctgctatacaacatgaacgactgctactccaagctcaaggagctggtgcccagcatcccccagaacaagaaggtgagcaagatggaaatcctgcagcacgtcatcgactacatcttggacctgcagatcgccctggactcgcatcccactattgtcagcctgcatcaccagagacccgggcagaaccaggcgtccaggacgccgctgaccaccctcaacacggatatcagcatcctgtccttgcaggcttctgaattcccttctgagttaatgtcaaatgacagcaaagcactgtgtggctgaataagcggtgttcatgatttcttttattctttgcacaacaacaacaacaacaaattcacggaatcttttaagtgctgaacttatttttcaaccatttcacaaggaggacaagttgaatggacctttttaaaaagaaaaaaaaaatggaaggaaaactaagaatgatcatcttcccagggtgttctcttacttggactgtgatattcgttatttatgaaaaagacttttaaatgccctttctgcagttggaaggttttctttatatactattcccaccatggggagcgaaaacgttaaaatcacaaggaattgcccaatctaagcagactttgccttttttcaaaggtggagcgtgaataccagaaggatccagtattcagtcacttaaatgaagtcttttggtcagaaattacctttttgacacaagcctactgaatgctgtgtatatatttatatataaatatatctatttgagtgaaaccttgtgaactctttaattagagttttcttgtatagtggcagagatgtctatttctgcattcaaaagtgtaatgatgtacttattcatgctaaactttttataaaagtttagttgtaaacttaacccttttatacaaaataaatcaagtgtgtttattgaatggtgattgcctgctttatttcagaggaccagtgctttgatttttattatgctatgttataactgaacccaaataaatacaagttcaaatttatgtagactgtataagattataataaaacatgtctgaagtcaaaaaaaaaaaaaaaaaaaaaaaaaaaaaaaaaaaaaaaa

SEQ ID NO: 31 - DNA, 2880 bp, homo sapiens
cactgcatgtgtattcgtgagttcgcggttgaacaactgttcctttactctgctccctgtctttgtgctgactgggttacttttttaaacactaggaatggtaatttctactcttctggacttcaaactaagaagttaaagagacttctctgtaaataaacaaatctcttctgctgtccttttgcatttggagacagctttatttcaccatatccaaggagtataactagtgctgtcattatgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatcttaaatggccccaggcatctgcctctggaaaactattgagccttgcatgtacttgaaggatggatgagtcagacacgattgagaactgacaaaggagccttgataatacttgacctctgtgaccaactgttggattcagaaatttaaacaaaaaaaaaaaaaaacacacacaccttggtaacatactgttgatatcaagaacctgtttagtttacattgtaacattctattgtaaaatcaactaaaattcagacttttagcaggactttgtgtacagttaaaggagagatggccaagccagggacaaattgtctattagaaaacggtcctaagagattctttggtgtttggcactttaaggtcatcgttgggcagaagtttagcattaatagttgttctgaaacgtgttttatcaggtttagagcccatgttgagtcttcttttcatgggttttcataatattttaaaactatttgtttagcgatggttttgttcgtttaagtaaaggttaatcttgatgatatacataataatctttctaaaattgtatgctgaccatacttgctgtcagaataatgctaggcatatgctttttgctaaatatgtatgtacagagtatttggaagttaagaattgattagactagtgaatttaggagtatttgaggtgggtggggggaagagggaaatgacaactgcaaatgtagactatactgtaaaaattcagtttgttgctttaaagaaacaaactgatacctgaattttgctgtgtttccattttttagagatttttatcatttttttctctctcggcattcttttttctcatactcttcaaaaagcagttctgcagctggttaattcatgtaactgtgagagcaaatgaataattcctgctattctgaaattgcctacatgtttcaataccagttatatggagtgcttgaatttaataagcagtttttacggagtttacagtacagaaataggctttaattttcaagtgaattttttgccaaacttagtaactctgttaaatatttggaggatttaaagaacatcccagtttgaattcatttcaaactttttaaatttttttgtactatgtttggttttattttccttctgttaatcttttgtattcacttatgctctcgtacattgagtacttttattccaaaactagtgggttttctctactggaaattttcaataaacctgtcattattgcttactttgattaaaaa

SEQ ID NO: 32 - DNA, 5228 bp, homo sapiens
cgggcgagggccgggcaggaggagcgggcgcggcgcgggcgaggctgggacccgagcgcgctcacttcgccgcaaagtgccaacttcccctggagtgccgggcgcgcaccgtccgggcgcgggggaaagaaaggcagcgggaatttgagatttttgggaagaaagtcggatttcccccgtccccttccccctgttactaatcctcattaaaaagaaaaacaacagtaactgcaaacttgctaccatcccgtacgtcccccactcctggcaccatgaaggcggccgtcgatctcaagccgactctcaccatcatcaagacggaaaaagtcgatctggagcttttcccctccccggatatggaatgtgcagatgtcccactattaactccaagcagcaaagaaatgatgtctcaagcattaaaagctactttcagtggtttcactaaagaacagcaacgactggggatcccaaaagacccccggcagtggacagaaacccatgttcgggactgggtgatgtgggctgtgaatgaattcagcctgaaaggtgtagacttccagaagttctgtatgaatggagcagccctctgcgccctgggtaaagactgctttctcgagctggccccagactttgttggggacatcttatgggaacatctagagatcctgcagaaagaggatgtgaaaccatatcaagttaatggagtcaacccagcctatccagaatcccgctatacctcggattacttcattagctatggtattgagcatgcccagtgtgttccaccatcggagttctcagagcccagcttcatcacagagtcctatcagacgctccatcccatcagctcggaagagctcctctccctcaagtatgagaatgactacccctcggtcattctccgagaccctctccagacagacaccttgcagaatgactactttgctatcaaacaagaagtcgtcaccccagacaacatgtgcatggggaggaccagtcgtggtaaactcgggggccaggactcttttgaaagcatagagagctacgatagttgtgatcgcctcacccagtcctggagcagccagtcatctttcaacagcctgcagcgtgttccctcctatgacagcttcgactcagaggactatccggctgccctgcccaaccacaagcccaagggcaccttcaaggactatgtgcgggaccgtgctgacctcaataaggacaagcctgtcattcctgctgctgccctagctggctacacaggcagtggaccaatccagctatggcagtttcttctggaattactcactgataaatcctgtcagtcttttatcagctggacaggagatggctgggaattcaaactttctgacccagatgaggtggccaggagatggggaaagaggaaaaacaaacctaagatgaattatgagaaactgagccgtggcctacgctactattacgacaaaaacatcatccacaagacagcggggaaacgctacgtgtaccgctttgtgtgtgacctgcagagcctgctggggtacacccctgaggagctgcacgccatgctggacgtcaagccagatgccgacgagtgatggcactgaaggggctggggaaaccctgctgagaccttccaaggacagccgtgttggttggactctgaattttgaattgttattctattttttattttccagaactcattttttaccttcaggggtgggagctaagtcagttgcagctgtaatcaattgtgcgcagttgggaaaggaaagccaggacttgtggggtgggtgggaccagaaattcttgagcaaattttcaggagagggagaagggccttctcagaagcttgaaggctctggcttaacagagaaagagactaatgtgtccaatcatttttaaaaatcatccatgaaaaagtgtcttgagttgtggacccattagcaagtgacattgtcacatcagaactcatgaaactgatgtaaggcaattaatttgcttctgtttttaggtctgggagggcaaaaaagaggtgggtgggatgaaacatgttttggggggggatgcactgaaaatctgagaactatttacctatcactctagttttgaagcaaagatggacttcagtggggaggggccaaaaccgttgttgtgttaaaatttattttattaaattttgtgccagtattttttttcttaaaaatcgtcttaagctctaaggtggtctcagtattgcaatatcatgtaagtttgtttttatttgccggctgaggattctgtcacaatgaaagaaaactgtttatatagaccccattggaaaagcaaaacgctctcactgagatcagggatcccaaattcatgggacttatataagaaggacaattaatgctgatttgggtacaggggaattatgtgtgtgaatgtcatctacaattaaaaaaaattagcacatccctttacttacttgttatcagtggattctcggggtttggacttaatgttgagctaagaagcattaagtctttgaactgaatgtattttgcatccctggttttggacgacagtaaacgtaggagcactgttgaagtcctggaagggagatcgaaggaggaagattgacttggttctttcttagtcctatatctgtagcatagatgacttggaataaaagctgtatgcatgggcattacccctcaggtcctaagaaataagtcctgaatgcatgtcgttccaaactaacactctgtaatttttcttttatgtcttattttccaagagtcctccattttttgcaccccctcaccgccaactctgttattcagtagagagaagtgtacggctttctgattggtgagtgaaaaagtaacttgagacacgacctaagttgaagagtttagacttgctgagttttagaagtgatggaaattaagagagcatttcaataaaatgtgacttggctgtctttggaagagaagtgcaaggctttcctttgaagaatttaaattagtccggtaggatgtcaggtgagactgtgtatgcaaaatgaatggcacaggtgatgccagggcctcttgcttgggtctgatgtcttggcacagggtaagtgaaggttaattccagaagagaggaatgacttgaaggcaaaggaaactaaggaaggaggttcagtgaggaaaataaggttgtccatgagatttgaatagatttttagttcccccaaggtttaaatacaaacatagtcaagcaaggtagtcatctttctgctggttgtgagggggaatctgaaaatggagttttagaggaaaagtcaacatctaactagtgaggaaaagtgcctaatacaattagaatctccctcactctatagttgcccagttgaaaggataaggaggaggggtggcttttatggacttccatgagagaaggaaagaaatatttcaggtaagcttctcagggctggccctttttgggatttggatgagaaattggaagtactaactactttctagcatatctttaagaaaattgattgttatttactcccagatcctcttgcagacccagaattatcaggaacatagctctgtgattcatgagtgtccccatactgatgaattggagcatccatatggaaagcaaaggcagaattatcccagctgtattattttgatcttttggatgcaggtgccttaatgaagctctcaaaatattttaggagctgctcagggagtgttgggtggaactgtttggactacattgttttctcttagattatgtgatttttgttgggcactggcaaaaggtgtgtgtgtgaatgtgtgcatgtgtgtgaatgttgtgtgtgtgtgtgtgtgtgtgtgtgtgtgtgtgtgtgtttgcagacatgcaaaactgcagctgaaataataccttagatttctaggtaagtctttccacatttcaataatgggtaagagtagaaccagggccgggtatcaattattgcttgctgtttgcaaccaggcataaaatcactttctcaaatcatccaccgttcctattaaatttatgccggaaactctccttctgtgagtataactcctgcagttcctatagcagataagatataagaaagtgcctcctagtgctcctccgcccgcttgtttgctaaaattccctttctctctaagtccaccattttcaagatttgtagatagtgtattagttaagacagctttgtcgatctggccagatgttttttctcctttgtccaaaggccagagaccatcccaggaagagtggtgggtggtttatacactggaaatgttgcgtttatgctttttaaaaacacacgttaacttcagaggaaggatgggcaaatctggtctagctgggtgaaacccttattttcccagagatgccttaacctttgttggttttggctttagggttcagagtcacttttgttcccttctccattctggagagggacttcccctacatagagccctgatttttgtggctgtggggattggaggtagcattcaaagatcagatgtgcttttcctcactttggagatgaacactctgggttttacagcattaacctgcctaaccttcatggtgagaaatacaccatctctcttctagtcatgctgtgcatgccgcttactctgttggggtctatataaatttgttgaactcttacctacattccaaagaagtttcaaggaaccataaatatatgtatacatatacatatataaaatatatatattaaaataaaattatcaggaatactgcctcagttattgaacttttttttttaagaatactttttttttaagctgagaagtatagggatgaaaaagatgttatattgtgtttgactattttccaacttgtattttcatataatttatattttttaaaagctgaaaatttagaagcaagatgaaaaaaaggaaaagcaggtgctttttaaaaatcagaactgaggtagcttagagatgtagcgatgtaagtgtcgatgtttttttaaaaaaaaatgcaaaaaaattcttatggcggagttttttgtttgtttattttagtagctgatgctggcacatcattttgctggagagttttttatatactgtagcctgatttcatattgtattttaaactgtgtgaaattaaaaacaaagaatttcattcataaaaaaaaaaaaaaaaaaaaaaaaaaaaaaaaaaaaaaaaaa

SEQ ID NO: 33 - DNA, 3663 bp, homo sapiens
ggcggcggcggcggcgcggactgggtgcgcggcgcagcgtcctgtgttggaatgtgcggctgccgcgagctcgcggcgcagcagcggagcgagcgccgccgaggcccggggccccagaccctggcggcggctgcggcagccgagacggcagggcgaggcccggaggcctgagcaccctctgcagccccactcctgggccttcttggtccacgacggccccagcacccaactttaccaccctcccccacctctcccccgaaactccagcaacaaagaaaagtagtcggagaaggagcggcgactcagggtcgcccgcccctcctcaccgaggaaggccgaatacagttatggccacccaggtaatggggcagtcttctggaggaggagggctgtttaccagcagtggcaacattggaatggccctgcctaacgacatgtatgacttgcatgacctttccaaagctgaactggccgcacctcagcttattatgctggcaaatgtggccttaactggggaagtaaatggcagctgctgtgattacctggtcggtgaagaaagacagatggcagaactgatgccggttggggataacaacttttcagatagtgaagaaggagaaggacttgaagagtctgctgatataaaaggtgaacctcatggactggaaaacatggaactgagaagtttggaactcagcgtcgtagaacctcagcctgtatttgaggcatcaggtgctccagatatttacagttcaaataaagatcttccccctgaaacacctggagcggaggacaaaggcaagagctcgaagaccaaaccctttcgctgtaagccatgccaatatgaagcagaatctgaagaacagtttgtgcatcacatcagagttcacagtgctaagaaattttttgtggaagagagtgcagagaagcaggcaaaagccagggaatctggctcttccactgcagaagagggagatttctccaagggccccattcgctgtgaccgctgcggctacaatactaatcgatatgatcactatacagcacacctgaaacaccacaccagagctggggataatgagcgagtctacaagtgtatcatttgcacatacacaacagtgagcgagtatcactggaggaaacatttaagaaaccattttccaaggaaagtatacacatgtggaaaatgcaactatttttcagacagaaaaaacaattatgttcagcatgttagaactcatacaggagaacgcccatataaatgtgaactttgtccttactcaagttctcagaagactcatctaactagacatatgcgtactcattcaggtgagaagccatttaaatgtgatcagtgcagttatgtggcctctaatcaacatgaagtaacccgccatgcaagacaggttcacaatgggcctaaacctcttaattgcccacactgtgattacaaaacagcagatagaagcaacttcaaaaaacatgtagagctacatgtgaacccacggcagttcaattgccctgtatgtgactatgcagcttccaagaagtgtaatctacagtatcacttcaaatctaagcatcctacttgtcctaataaaacaatggatgtctcaaaagtgaaactaaagaaaaccaaaaaacgagaggctgacttgcctgataatattaccaatgaaaaaacagaaatagaacaaacaaaaataaaaggggatgtggctggaaagaaaaatgaaaagtccgtcaaagcagagaaaagagatgtctcaaaagagaaaaagccttctaataatgtgtcagtgatccaggtgactaccagaactcgaaaatcagtaacagaggtgaaagagatggatgtgcatacaggaagcaattcagaaaaattcagtaaaactaagaaaagcaaaaggaagctggaagttgacagccattctttacatggtcctgtgaatgatgaggaatcttcaacaaaaaagaaaaagaaggtagaaagcaaatccaaaaataatagtcaggaagtgccaaagggtgacagcaaagtggaggagaataaaaagcaaaatacttgcatgaaaaaaagtacaaagaagaaaactctgaaaaataaatcaagtaagaaaagcagtaagcctcctcagaaggaacctgttgagaagggatctgctcagatggaccctcctcagatggggcctgctcccacagaggcggttcagaaggggcccgttcaggtggagctgccacctcccatggagcatgctcagatggagggtgcccagatacggcctgctcctgacgagcctgttcagatggaggtggttcaggaggggcctgctcagaaggagctgctgcctcccgtggagcctgctcagatggtgggtgcccaaattgtacttgctcacatggagctgcctcctcccatggagactgctcagacggaggttgcccaaatggggcctgctcccatggaacctgctcagatggaggttgcccaggtagaatctgctcccatgcaggtggtccagaaggagcctgttcagatggagctgtctcctcccatggaggtggtccagaaggagcctgttcagatagagctgtctcctcccatggaggtggtccagaaggaacctgttaagatagagctgtctcctcccatagaggtggtccagaaggagcctgttcagatggagttgtctcctcccatgggggtggttcagaaggagcctgctcagagggagccacctcctcccagagagcctccccttcacatggagccaatttccaaaaagcctcctctccgaaaagataaaaaggaaaagtctaacatgcagagtgaaagggcacggaaggagcaagtccttattgaagttggcttagtgcctgttaaagatagctggcttctaaaggaaagtgtaagcacagaggatctctcaccaccatcaccaccactgccaaaggaaaatttaagagaagaggcatcaggagaccaaaaattactcaacacaggtgaaggaaataaagaagcccctcttcagaaagtaggagcagaagaggcagatgagagcctacctggtcttgctgctaatatcaacgaatctacccatatttcatcctctggacaaaacttgaatacgccagagggtgaaactttaaatggtaaacatcagactgacagtatagtttgtgaaatgaaaatggacactgatcagaacacaagagagaatctcactggtataaattcaacagttgaagaaccagtttcaccaatgcttcccccttcagcagtagaagaacgtgaagcagtgtccaaaactgcactggcatcacctcctgctacaatggcagcaaatgagtctcaggaaattgatgaagatgaaggcatccacagccatgaaggaagtgacctaagtgacaacatgtcagagggtagtgatgattctggattgcatggggctcggccagttccacaagaatctagcagaaaaaatgcaaaggaagccttggcagtcaaagcggctaagggagattttgtttgtatcttctgtgatcgttctttcagaaagggaaaagattacagcaaacacctcaatcgccatttggttaatgtgtactatcttgaagaagcagctcaagggcaggagtaatgaaactttgaacaaggtttca

SEQ ID NO: 34 - DNA, 668 bp, homo sapiens
cggggacccggctgcggtggctgcgggactgacgagaaactactaaagttcctggggaagcaaagtagaatttcataagaacaaaatggatggagaggagaaaacctatggtggctgtgaaggacctgatgccatgtatgtcaaattgatatcatctgatggccatgaatttattgtaaaaagagaacatgcattaacatcaggcacgataaaagccatgttgagtggcccaggtcagtttgctgagaacgaaaccaatgaggtcaattttagagagataccttcacatgtgctatcgaaagtatgcatgtattttacgtacaaggttcgctacactaacagctccaccgagattcctgaattcccaattgcacctgaaattgcactggaactgctgatggctgcgaacttcttagattgttaaataaaataaattataataaactgttaactcttttcagtatttaatacctgtagttcagttagtaactttttcatatatagcatgttgcctgtatgcagttgaactatataaagttcattgcaaagcagattatcttgtttttttgcatagcaatcaaagttgaaatttgtttgctacatcaacaaattaaggacattttcacaaactgagaaataaacaaatatgccaattaaaaaaaaaaaaa

SEQ ID NO: 35 - DNA, 1306 bp, homo sapiens
cgcgcttggccttgcccgcgcccgctcgcctcgtctcgcccggcctccccgcgtcgcctcgtcgcctgttccgcgccaggcatggcccccagcactgtggccgtggagctgctcagccccaaagagaaaaaccgactgcggaagccggtggtggagaagatgcgccgcgaccgcatcaacagcagcatcgagcagctgaagctgctgctggagcaggagttcgcgcggcaccagcccaactccaagctggagaaggccgacatcctggagatggctgtcagctacctgaagcacagcaaagccttcgtcgccgccgccggccccaagagcctgcaccaggactacagcgaaggctactcgtggtgcctgcaggaggccgtgcagttcctgacgctccacgccgccagcgacacgcagatgaagctgctgtaccacttccagcggcccccggccgcgcccgccgcgcccgccaaggagcccaaggcgccgggcgccgcgcccccgcccgcgctctccgccaaggccaccgccgccgccgccgccgcgcaccagcccgcctgcggcctctggcggccctggtgacccggcgggacctgcgggcgcgcggcccgacgaccagagggcgagcctgctcctctcgcctgtagggaagcgccttcccgccgtcgtccgccccgggcttggacgcgcccttctccggaaggctctggccccaagctggccggcccgcaggagccccattctcagagaatgtgtgtgcagagtccctgccgttttaggacaatcagggcccatcttctgccaagtgtctgaccccatggggttgttctgtgtttgcatttaagcaagtgacttctgggaagtccccggccgcccggggttctatgatatttgtagtgccggggctcgcacactgctgcccccagcctgtagaggactttcttcagggcccgtagctgctgggcgtacccctggcaggcgggctgtgccgcgggcacatttgccttttgtgaaggccgaactcgagctgtatcctcataggaaacagtgatcaccccggacgggcgtccaggaccctgagggccatggccaaaaggctcctgagtgtgcctggtggtctggctggggctcacggtgggctgtctggggagggtgggtgcctccactatgatccttaaaggattcctctgtgtgggtggatgcgtgtgggcacgactttgtactcagaaattgaactctcagtcacgtggaagccacgggactgctccgaagccgccataataaaatctgattgttcagcccccaa

SEQ ID NO: 36 - DNA, 1914 bp, homo sapiens
gcagccaggcgcgcactgcacagctctcttctctcgccgccgcccgagcgcacccttcagcccgcgcgccggccgtgagtcctcggtgctcgcccgccggccagacaaacagcccgcccgaccccgtcccgaccctggccgccccgagcggagcctggagcaaaatgatgcttcaacacccaggccaggtctctgcctcggaagtgagtgcttctgccatcgtcccctgcctgtcccctcctgggtcactggtgtttgaggattttgctaacctgacgccctttgtcaaggaagagctgaggtttgccatccagaacaagcacctctgccaccggatgtcctctgcgctggaatcagtcactgtcagcgacagacccctcggggtgtccatcacaaaagccgaggtagcccctgaagaagatgaaaggaaaaagaggcgacgagaaagaaataagattgcagctgcaaagtgccgaaacaagaagaaggagaagacggagtgcctgcagaaagagtcggagaagctggaaagtgtgaatgctgaactgaaggctcagattgaggagctcaagaacgagaagcagcatttgatatacatgctcaaccttcatcggcccacgtgtattgtccgggctcagaatgggaggactccagaagatgagagaaacctctttatccaacagataaaagaaggaacattgcagagctaagcagtcgtggtatgggggcgactggggagtcctcattgaatcctcattttatacccaaaaccctgaagccattggagagctgtcttcctgtgtacctctagaatcccagcagcagagaaccatcaaggcgggagggcctgcagtgattcagcaggcccttcccattctgccccagagtgggtcttggaccagggcaagtgcatctttgcctcaactccaggatttaggccttaacacactggccattcttatgttccagatggcccccagctggtgtcctgcccgcctttcatctggattctacaaaaaaccaggatgcccaccgttagattcaggcagcagtgtctgtacctcgggtgggagggatggggccatctccttcaccgtggctaccattgtcactcgtaggggatgtggagtgagaacagcatttagtgaagttgtgcaacggccagggttgtgctttctagcaaatatgctgttatgtccagaaattgtgtgtgcaagaaaactaggcaatgtactcttccgatgtttgtgtcacacaacactgatgtgacttttatatgctttttctcagatctggtttctaagagttttggggggcggggctgtcaccacgtgcagtatctcaagatattcaggtggccagaagagcttgtcagcaagaggaggaacagaattctcccagcgttaacacaaaatccatgggcagcatgatggcaggtcctctgttgcaaactcagttccaaagtcacaggaagaaagcagaaagttcaacttccaaagggttaggactctccactcaatgtcttaggtcaggagttgtgtctaggctggaagagccaaagaaatattccattttcctttccttgtggttgaaaccacagtcagtggagagatgtttggaacacagtcagtggagctggtggtaccaggtttagcattattggatgtcaaaagcattttttttgtcatgtagctgttttaagaaatctggcccagggtgtttgcagctgtgagaagtcactcacactggccacaaggacgctggctactgtctattaaaattctgatgtttctgtgaaattctcagagtgtttaattgtactcaatggtatcattacaattttctgtaagagaaaatattacttatttatcctagtattcctaacctgtcagaataataaatattgtggtaaaa

SEQ ID NO: 37 - DNA, 4789 bp, homo sapiens
ctctcgctccggagccgctgcacatttcggaatcttctgcggcttgtccatagtgtgaataaaaactaaatcacatctataattctactgaactggtcatacagacgctgccatatatgtcacagtacactgaaaaggagccagcagcaatggaccaagaatctggtaaggctgtctggcccaaaccagcaggagggtatcagacaattacaggcaggagatatggaagaagacatgcttatgtcagttttaaaccatgtatgaccagacatgaaagaagcttaggtcgggctggtgatgactatgaagtgttggaactagatgatgttccaaaggaaaattcctcaggttccagtcctttggatcaagttgattcttctttaccaagtgaacctatatttgaaaaaagtgaaacagaaattcccacttgtggttcagcattgaatcaaaccactgagagcagtcaatcctttgttgcagtacatcacagtgaggaaggcagggataccttaggaagcagtacaaatcttcataatcactctgagggagagtatattccaggagcttgtagtgcttcaagtgtccaaaatggaattgcattggttcatacagactcttatgatccagatggcaaacatggagaagataatgaccatcttcaactttctgcagaagtcgtggaaggtagtagataccaggaatcattaggcaatacagtatttgagttggaaaacagagaggcagaggcatacactggtctttcaccaccagttccctcatttaactgtgaagtaagagatgagtttgaagagttagattctgtaccattagtgaaaagttctgctggtgatactgagtttgtccatcagaatagccaggaaattcagaggtcttctcaagatgaaatggttagtacgaaacaacaaaataatactagccaggaaagacagacagaacattcacctgaagatgcagcctgtggtccagggcatatttgtagtgaacgaaataccaatgatagggaaaagaaccatggaagttctcctgaacaggtagtgaggccaaaagttagaaaactgataagttcaagccaggtggaccaagaaacaggttttaataggcatgaggcgaaacaaagaagtgttcaaagatggagagaggctttggaagttgaggaaagtggctcagatgacctcttaataaaatgtgaagaatatgatggagagcatgactgtatgttcttggatccaccatactcaagagttattacacaaagggaaacagaaaataaccaaatgacatcagaaagtggagccacagcgggaaggcaagaagtggataacaccttttggaatggctgtggagattattaccaactctatgacaaagatgaagatagttctgaatgcagtgatggggaatggtctgcttctttgcctcatcgattttctggtacagaaaaagatcaatcctcaagtgatgaaagctgggagactctgccaggaaaagatgagaatgaacctgagctacaaagtgatagcagtggccctgaagaagaaaaccaagaattatctcttcaggaaggggaacagacatccttggaagagggagaaattccttggttacagtacaatgaagtcaatgaaagcagcagtgatgagggaaatgaacctgccaatgaatttgcacagccagctttcatgttggatggtaacaataacctggaggatgactccagtgtgagtgaagacttagatgtggattggagcatatttgatggctttgcagatggactaggagttgctgaagctatttcatatgtggatcctcagttccttacatacatggcactagaagaacgcttagcccaggctatggagactgctctggcccatttagagtctcttgcagtggatgttgaggtggccaatccaccagctagtaaggaaagcattgatggtcttccagagacccttgttcttgaagatcacactgctattggtcaggaacaatgctgtccaatctgttgcagtgagtatattaaggatgatatagcaacagagttgccctgtcaccatttctttcacaaaccttgtgtctcaatttggctacaaaagtcgggaacatgccctgtgtgccgccgtcatttcccacctgcggttattgaagcatctgcagctccttcctctgagcctgatcctgatgccccaccttcgaatgacagtattgcagaagcaccctaaaccttgacagttgaaatgagatcagtgtatcaaagtaaatctgcaaattccttctaaatttcatgtgcaaataattatatataaatatatttaaaaatgctatatatagtatatgccatagtttagaaagaatattaacctttctaaactaaatttaggtttgcagaaagtattaaacatttttaagctgaatgttaagacagtgcatccattttctttagttgaatatgtttgtattaattgtaaagccaagcttatcagttgactctctccagaataaataatcatctgtgtggcatacgttattggctttgtctgtaatactgccactaagtgattataattaagctgtcatgtttgcatcaaatgtaaagactgtgttcacaaccgtagtaaaatttggtttcattggaaatgaaacaaattctaaagtatgctttttcactagtccctttgattttgctatatcataacctcttggttcatatgctgagaaatttttcagaaagtccatttttgtttaaaattagaacttttcagaatgccaaatgaaggctaaaattttggccagaacattacaaaagttttaaatcgtagacgtaactccccctgaaataaagttaggtagtaaaatccttaatgaaaccagtggatgtgcttaacgtaaggttagtaaagcatacaaagaatctagtgtgctcagggcttggtacaatgagctgaattagatggccttatgaaactctttctaacctcttacccaacctgtttctccttggttaaaattatacttgaaggcccagaacactcatggcacatttgtttaatattgcttatagttagtttaaggtaattttgcttctacagtattttggaaggtctgaaaacttgcacagggtcatctttgtaattatataaccccaaactaagatgcacaatgtctccttcaggtgatcacacacagtggacgagtatgtgcaaacatggacataatagttcacttacaaatgtgatttgatgttaacactagagaatgatgactgtagaacatttgagcaagtaaaatagtaaagcacatagtgagtgtatgtccgtctaactggtacattgataatttagtttgggcacataaaaggaatatttatatggcttcccaaatgcagagttacatcttattcgtgtatttctctgagtatttatatcccgtctccttttttcattcttaaaaataaatgaattttcactgttggcacatatgaggcttaaatataaggaacataacacttgcattctaatttttgcatatattgtaaatgtgtctggtatttacagcaaaatactgtgtatccttttatgggtaaaacaaaagtgaacattgcatgcatgtaatgtgatgaatttgtaatttaggagttctttggggcttctgtgacttgggtaatgcttacattcaggccttaatgttgcattagctagcatgttttccctctgatgtatatagtcactgttgtataaactaatctttgcttgttttctactctgtgatctttccatatcatatttcattaatgatcagttagtgtcaaggagtcaaaacagattaaaattaatttcatgtgtatatggtggaaatttgtggctagtgtgatttttgtttgtttccttttaagtactgttgatcagttgtgacacttactggttaaacttacgttgctaaagatttctctataataagccacacattatatttagactatattaagggaccttggttttcttctagatagcagctgtcccaaagaaaatatttcttctttgtctgttaagatttagctattatctgccagttgttaagaggttttggttccaaactcaaccagcaatgttgagagctgaacttaagatagctgttgtactttttgctttccatctgttactgtccttcattcttggctccctactatctataaacagctgctgtgaagaagaaaagttgaataagagttggcttaaattttaaaaaagaaaaagaaaattgaggttttaggattttcatggtaacaagctctggtataagctaaggctggcaagttcagatactaaaatattatttgatcatatcttggatccttttgaaaaagttaagactatatgaaggtaaattagaaataagtatgaatattaataaaatagcatttatcttatttctctattttatgttgtgacttaacctaattttatttttttaacattttcttatttcttataatatgaatgctgatatttaaaggtagatctatgtggtattctttgtgtttcttaattgtttaactcttaagattatttgtgatctggatttatgtatttgttagatacatacgaattgttaaaatggaatgcaagtttttcaaaagcccaggtctaaatgtaatggttggtttattgttctataaccccagcccatcattttctgtgtaaatcataaacaataaacagaatatactcggtggtcaaaaaaaaaaaaaaaaagaaaaaaaaaaaaaaaa

SEQ ID NO: 38 - DNA, 3448 bp, homo sapiens
attaaccttctcttagtcttcaacctaagtacttgaatgtcaagtaccctccaaccctcaatgtcccaagacttttaagagcggaaggtaccgatgagttccatcctttactagggtcaccaaggaaggcatgggtatatggaaatttttattattattccatctgaatatcattttctagagaataggagcttttgttctgaagggctgccggcttccttctgggatctagcagccagggttagatcacaggtgtcactttcaggcgagtagttagcaacggtatcgctagcaactgagccgacccctgcagccagaggtttgcagtgggtagtgtgtattccagaaagggccctgacatgtgaaaggaaggaatgtgccctaatattctacagttgttttatcgttgctactgattaggtccatggagggaagcccatccctgagacgcatgacagtgatgcgggagaagggccggcgccaggctgtcaggggcccggccttcatgttcaatgaccgcggcaccagcctcaccgccgaggaggagcgcttcctcgacgccgccgagtacggcaacatcccagtggtgcgcaagatgctggaggagtccaagacgctgaacgtcaactgcgtggactacatgggccagaacgcgctgcagctggctgtgggcaacgagcacctggaggtgaccgagctgctgctcaagaaggagaacctggcgcgcattggcgacgccctgctgctcgccatcagcaagggctacgtgcgcatcgtagaggccatcctcaaccaccctggcttcgcggccagcaagcgtctcactctgagcccctgtgagcaggagctgcaggacgacgacttctacgcttacgacgaggacggcacgcgcttctcgccggacatcacccccatcatcctggcggcgcactgccagaaatacgaagtggtgcacatgctgctgatgaagggtgccaggatcgagcggccgcacgactatttctgcaagtgcggggactgcatggagaagcagaggcacgactccttcagccactcacgctcgaggatcaatgcctacaaggggctggccagcccggcttacctctcattgtccagcgaggacccggtgcttacggccctagagctcagcaacgagctggccaagctggccaacatagagaaggagttcaagaatgactatcggaagctctccatgcaatgcaaagactttgtagtgggtgtgctggatctctgccgagactcagaagaggtagaagccattctgaatggagatctggaatcagcagagcctctggaggtacacaggcacaaagcttcattaagtcgtgtcaaacttgccattaagtatgaagtcaaaaagtttgtggctcatcccaactgccagcagcagctcttgacgatctggtatgagaacctctcaggcctaagggagcagaccatagctatcaagtgtctcgttgtgctggtcgtggccctgggccttccattcctggccattggctactggatcgcaccttgcagcaggctggggaaaattctgcgaagcccttttatgaagtttgtagcacatgcagcttctttcatcatcttcctgggtctgcttgtgttcaatgcctcagacaggttcgaaggcatcaccacgctgcccaatatcacagttactgactatcccaaacagatcttcagggtgaaaaccacccagtttacatggactgaaatgctaattatggtctgggttcttggaatgatgtggtctgaatgtaaagagctctggctggaaggacctagggaatacattttgcagttgtggaatgtgcttgactttgggatgctgtccatcttcattgctgctttcacagccagattcctagctttccttcaggcaacgaaggcacaacagtatgtggacagttacgtccaagagagtgacctcagtgaagtgacactcccaccagagatacagtatttcacttatgctagagataaatggctcccttctgaccctcagattatatctgaaggcctttatgccatagctgttgtgctcagcttctctcggattgcgtacatcctccctgcaaatgagagctttggccccctgcagatctctcttggaaggactgtaaaggacatattcaagttcatggtcctctttattatggtgttttttgcctttatgattggcatgttcatactttattcttactaccttggggctaaagttaatgctgcttttaccactgtagaagaaagtttcaagactttattttggtcaatatttgggttgtctgaagtgacttccgttgtgctcaaatatgatcacaaattcatagaaaatattggatacgttctttatggaatatacaatgtaactatggtggtcgttttactcaacatgctaattgctatgattaatagctcatatcaagaaattgaggatgacagtgatgtagaatggaagtttgctcgttcaaaactttggttatcctattttgatgatggaaaaacattacctccacctttcagtctagttcctagtccaaaatcatttgtttatttcatcatgcgaattgttaactttcccaaatgcagaaggagaaggcttcagaaggatatagaaatgggaatgggtaactcaaagtccaggttaaacctcttcactcagtctaactcaagagtttttgaatcacacagttttaacagcattctcaatcagccaacacgttatcagcagataatgaaaagacttataaagcggtatgttttgaaagcacaagtagacaaagaaaatgatgaagttaatgaaggtgaattaaaagaaatcaagcaagatatctccagccttcgttatgaacttttggaagacaagagccaagcaactgaggaattagccattctaattcataaacttagtgagaaactgaatcccagcatgctgagatgtgaatgatgcagcaacctggatttggctttgactatagcacaaatgtgggcaataatatttctaagtatgaaatacttgaaaaactatgatgtaaatttttagtattaactacctttatcatgtgaacctttaaaagttagctcttaatggttttattgttttatcacatgaaaatgcattttatttgtctgctttgacattacagtggcataccattgtgttgaaaagcccaatattactatattattgaaacttttattcattttagagtaaactccacatctttgcactacctgtttgcctccaagagactatcagttccttggggacagggaccatgtcttattcatctttgtgtctccagcatctagtacagtgcctggtatatagtaggtgctcaataaatgttgaaaccaactgaactgccaacaaaataaaaataaaaagtcttcactatgtagcataaaaaaaaaaaaaaaaaaa

SEQ ID NO: 39 - DNA, 1866 bp, homo sapiens
cgttcctcggcgccgccggggccccagagggcagcggcagcaacagcagcagcagcagcagcgggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcagcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggagctcaggtggccccagttcaatctcccgctgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctgaaaaaaaaaaaaaaaa

SEQ ID NO: 40 - DNA, 1986 bp, homo sapiens
agtctgggttggactggcggccgtggagtttgtgacatacgaggtgacacccctcgagtcacttcccttcaactccagctggagcgcctgcttggctttgggttcgttctgcagccttcgccccgctcctagcctcagggccggactccagcgcagagcccagcccagcgcagcctgccagcagccacccagccgcccagccgcccagccccgcacgaaacccggccagagcttcctagcagcccgagccatgaacaccgaaatgtatcagacccccatggaggtggcggtctaccagctgcacaatttctccatctccttcttctcttctctgcttggaggggatgtggtttccgttaagctggacaacagtgcctccggagccagcgtggtggccatagacaacaagatcgaacaggccatggatctggtgaagaatcatctgatgtatgctgtgagagaggaggtggagatcctgaaggagcagatccgagagctggtggagaagaactcccagctagagcgtgagaacaccctgttgaagaccctggcaagcccagagcagctggagaagttccagtcctgtctgagccctgaagagccagctcccgaatccccacaagtgcccgaggcccctggtggttctgcggtgtaagtggctctgtcctcagggtgggcagagccactaaacttgttttacctagttctttccagtttgtttttggctccccaagcatcatctcacgaggagaactttacacctagcacagctggtgccaagagatgtcctaaggacatggccacctgggtccactccagcgacagacccctgacaagagcaggtctctggaggctgagttgcatggggcctagtaacaccaagccagtgagcctctaatgctactgcgccctgggggctcccagggcctgggcaacttagctgcaactggcaaaggagaagggtagtttgaggtgtgacaccagtttgctccagaaagtttaaggggtctgtttctcatctccatggacatcttcaacagcttcacctgacaacgactgttcctatgaagaagccacttgtgttttaagcagaggcaacctctctcttctcctctgtttcgtgaaggcaggggacacagatgggagagattgagccaagtcagccttctgttggttaatatggtataatgcatggctttgtgcacagcccagtgtgggattacagctttgggatgaccgcttacaaagttctgtttggttagtattggcatagtttttctatatagccataaatgcgtatatatacccatagggctagatctgtatcttagtgtagcgatgtatacatatacacatccacctacatgttgaagggcctaaccagccttgggagtattgactggtcccttacctcttatggctaagtctttgactgtgttcatttaccaagttgacccagtttgtcttttaggttaagtaagactcgagagtaaaggcaaggaggggggccagcctctgaatgcggccacggatgccttgctgctgcaaccctttccccagctgtccactgaaacgtgaagtcctgttttgaatgccaaacccaccattcactggtgctgactacatagaatggggttgagagaagatcagtttgggcttcacagtgtcatttgaaaacgttttttgttttgttttgtaattattgtggaaaactttcaagtgaacagaaggatggtgtcctactgtggatgagggatgaacaaggggatggctttgatccaatggagcctgggaggtgtgcccagaaagcttgtctgtagcgggttttgtgagagtgaacactttccactttttgacaccttatcctgatgtatggttccaggatttggattttgattttccaaatgtagcttgaaatttcaataaactttgctctgtttttctaaaaataaaaaaaaaaaaaaaaaaaaaaaaaaaaaaaaaaaa

SEQ ID NO: 41 - DNA, 3302 bp, homo sapiens
cggtgctgccgccgttgccgggagccgcggagacaagtcattacgttttcatttctcacaactgggctgagcacaactgaaccatgggggaacacagtccagacaacaacatcatctactttgaggcagaggaagatgagctgacccccgatgataaaatgctcaggtttgtggataaaaacggactggtgccttcctcatctggaactgtttatgataggaccactgttcttattgagcaggaccctggcactttggaggatgaagatgacgacggacagtgcggagaacacttgccttttctagtagggggtgaagagggctttcacctgatagatcatgaagcaatgtcccagggttatgtgcagcacattatctcaccagatcagattcatttgacaataaaccctggttccacacccatgccaagaaatattgaaggtgcaaccctcactctgcagtcggaatgtccggaaacaaaacgtaaagaagtaaagcggtaccaatgtacctttgagggctgtccccgcacctacagcacagcaggcaacctgcgaacccaccagaagactcaccgaggagagtacacctttgtctgtaatcaggagggctgtggcaaagccttccttacctctcacagcctcaggatccacgtgcgagtgcacacgaaggagaagccatttgagtgtgacgtgcagggctgtgagaaggcattcaacacactgtacaggctgaaagcacatcagaggcttcacacagggaaaacgtttaactgtgaatctgaaggctgcagcaaatacttcaccacactcagtgatctgaggaagcacattcgaactcatacaggggaaaagccatttcggtgcgatcacgatggctgtggaaaagcatttgcagcaagccaccaccttaaaactcacgttcgtacacatactggtgaaagacccttcttctgccccagtaatggctgtgagaaaacattcagcactcaatacagtctcaaaagtcacatgaaaggtcatgataacaaaggacactcatacaatgcacttccacaacacaatggatcagaggatacaaatcactcactttgtctaagtgacttgagccttctgtccacagattctgaattgcgagaaaattccagtacgacccagggccaggacctcagcacaatttcaccagcaatcatctttgaatcaatgttccagaattcagatgatacggcaattcaggaagatcctcaacagacagcttccttgactgaaagttttaatggtgatgcagagtcagtcagtgatgttccgccatccacaggaaattcagcatctttatctcttccacttgtactgcaacctggcctctccgagccaccccagcctctactacctgcctcagctccgtctgctcctccgcctgctccctccctaggacctggctcccagcaagctgcatttggcaacccccctgctctcttacaacctccagaagtgcctgttccccacagcacacagtttgctgctaatcatcaagagtttcttccgcacccccaggcaccgcagcccattgtaccaggactttctgttgttgctggggcttctgcatcagcagcggcagtggcatcagctgtggcagcaccagccccaccacaaagtactactgagcccctgccagccatggtccagactctgcccctgggtgccaactctgtcctaactaataatcccacaataaccatcaccccaactcccaacacagctatcctgcagtccagcctagtcatgggagaacagaacttacaatggatattaaatggtgccaccagttctccacaaaaccaagaacaaattcagcaagcatctaaagttgagaaggtgttttttaccactgcagtaccagtagccagtagcccagggagctctgtccagcagattggcctcagtgttcctgtgatcatcatcaaacaagaagaggcatgtcagtgtcagtgtgcatgccgggactctgcaaaggagcgggcatccagcaggagaaagggctgctcctccccaccccctccagagccgagcccccaggctcctgatgggcccagcctgcagctcccagcgcagactttctcttcagcccctgttcccgggtcatcatcctctaccttgccctcctcctgtgagcaaagccgacaagcagagactccttcagaccctcagacagaaacattaagtgccatggatgtgtcagagtttctatccctccagagcctggacaccccgtccaatctgattcccattgaagcactactgcagggggaggaggagatgggcctcaccagcagcttctccaagtgaagggcccatgtgtgctcacctctgggaaaagcgggtgagcaggaggcatgaggtacaatgcctgccatcatgggtcagaaatttgaaggatgaagaaatctactgtttgaaatcctcacctttcagacgtattttctttattcacatcccaggagcatccattttaaggaactattctttggaaaaaaacaaaaaacaaaaaaaacaacaaaaaaagctaagttataagtgaactgtttggctgcactgtatgtcacttttgcttgttgtcatgtgaacttggaaactaaggttactcgtgtgcataaaaattctaaatgaaagggtgtggtttccatcaatctgatgctgcccatcgcttgcactggggtctttgtggatcgggcaggagttttcagtgtgttgggtgttgctccttcctatgtgtcttttgaatctgaggctgacatttgcttggaaggccagacccttgctccatcagagagggcagtggcaaaggccagtgaggcagctgtgagttggacagggttcaggtgagatggtgttgtcatttgtgcttagtgttggtggtgctcagggtggataacacgggtcgttctgcagcccgcttcagcacaaataggcagcttaaggcctggctcacaggctgtggggttgatctggctctgcagaggccctaggcagcttgttgactgctgtctgttgatgacgtgtgtgcaaagcaggctctagcaacatgatcactgtccttgccttcctggttctttctctcggttggttgccagggcttgcagatcgcagtgaattttccttggggaacatcgctgttttgtcctagagtgaacttgtggcttatggccagtgctgtttggtggtctgccttctttttaatggtattttcttcctcagagcagaagggctgcattttgcttatcagaagaaggtgcagatttaagg

SEQ ID NO: 42 - DNA, 2104 bp, homo sapiens
acgtagcgcggcgctcggaactgacctactaacacacatctctccgcgcggccacggcgcccgcggacccggcgcgcccgcccgcctcccgcgccgcgccctcgccgccgcccgcctcccgccgcggccccggaggcccggcccggcccgagccccgagcgccggcggcccgactcccggccgcccctttctttctcctcgccggcccgagagcaggaacacgataacgaaggaggcccaacttcattcaataaggagcctgacggatttatcccagacggtagaacaaaaggaagaatattgatggattttaaaccagagtttttaaagagcttgagaatacggggaaattaatttgttctcctacacacatagatagggtaaggttgtttctgatgcagctgagaaaaatgcagaccgtcaaaaaggagcaggcgtctcttgatgccagtagcaatgtggacaagatgatggtccttaattctgctttaacggaagtgtcagaagactccacaacaggtgaggagctgcttctcagtgaaggaagtgtggggaagaacaaatcttctgcatgtcggaggaaacgggaattcattcctgatgaaaagaaagatgctatgtattgggaaaaaaggcggaaaaataatgaagctgccaaaagatctcgtgagaagcgtcgactgaatgacctggttttagagaacaaactaattgcactgggagaagaaaacgccactttaaaagctgagctgctttcactaaaattaaagtttggtttaattagctccacagcatatgctcaagagattcagaaactcagtaattctacagctgtgtactttcaagattaccagacttccaaatccaatgtgagttcatttgtggacgagcacgaaccctcgatggtgtcaagtagttgtatttctgtcattaaacactctccacaaagctcgctgtccgatgtttcagaagtgtcctcagtagaacacacgcaggagagctctgtgcagggaagctgcagaagtcctgaaaacaagttccagattatcaagcaagagccgatggaattagagagctacacaagggagccaagagatgaccgaggctcttacacagcgtccatctatcaaaactatatggggaattctttctctgggtactcacactctcccccactactgcaagtcaaccgatcctccagcaactccccgagaacgtcggaaactgatgatggtgtggtaggaaagtcatctgatggagaagacgagcaacaggtccccaagggccccatccattctccagttgaactcaagcatgtgcatgcaactgtggttaaagttccagaagtgaattcctctgccttgccacacaagctccggatcaaagccaaagccatgcagatcaaagtagaagcctttgataatgaatttgaggccacgcaaaaactttcctcacctattgacatgacatctaaaagacatttcgaactcgaaaagcatagtgccccaagtatggtacattcttctcttactcctttctcagtgcaagtgactaacattcaagattggtctctcaaatcggagcactggcatcaaaaagaactgagtggcaaaactcagaatagtttcaaaactggagttgttgaaatgaaagacagtggctacaaagtttctgacccagagaacttgtatttgaagcaggggatagcaaacttatctgcagaggttgtctcactcaagagacttatagccacacaaccaatctctgcttcagactctgggtaaattactactgagtaagagctgggcatttagaaagatgtcatttgcaatagagcagtccattttgtattatgctgaattttcactggacctgtgatgtcatttcactgtgatgtgcacatgttgtctgtttggtgtctttttgtgcacagattatgatgaagattagattgtgttatcactctgcctgtgtatagtcagatagtccatgcgaaggctgtatatattgaacattatttttgttgttctattataaagtgtgtaagttaccagtttcaataaaggattggtgacaaacacagaaaaaaaaaaaaaaaaaaaaa

SEQ ID NO: 43 - DNA, 4776 bp, rattus norvegicus
gcacgagccgaggcttcctatgccggctggagttaggatgccacggcccattatttgcaccgactaacccggagttcctcctcggccctgggaaggactggcggcggcggctggagcagacagagcagcaggctctcttctccgaggggaggggggggcgtcgaagcgccctcgctgggctctagccctcacccacggtcctcaagccgtcacctcggtggcctgcttcctttttcttctccccgcgactccgggcccgaccgcctctgggatttgacccgcgcgccccggatgctccgagctgcccagacgcgcccccccccctccagcctcatcgtgctgattgacccgtgaggggacaagagaaacccaccccccaacctccgggcctcgggatcctgctctggagctgccagggagccgatgatgtccacgcagacccggcttctgacactgtccacgcagccgggggaaggtggccggccgcagccggccgagccttaggcgcacaaagcgcgagcccgccgacgactttgccgctcgctccgccgctctttgtctgcacttggggcgcacgcccgactctgggatttcgctgcgagagcgggctgggggggccgggggcgagctctcggttccccggccgccccccgctcgggctcgcctcccccctcccccgcacgtccccggctcgggctctcagcgcttccaagctctcggaatcgcgacccctccctgccatgtatccgcaaggcagacatccggcaccccatcaacccgggcagccaggatttaaattcactgtggccgagtcctgtgacaggatcaaagacgaattccagttcctgcaagctcagtatcacagcctcaaagtggagtacgacaagctggcgaacgagaagacagagatgcagcgccattatgtgatgtactatgagatgtcctacggcttgaatatcgaaatgcacaagcagacagagatcgcgaagagactgaacacgattctagcccagatcatgccttttttgtcacaggagcatcagcagcaagtggcgcaggctgtggaacgcgccaagcaggtcaccatgacggagctgaacgccatcatcgggcagcagcaactccaggcacagcacctctcccatgccacacatggtcccccggtccagctgccaccccacccgtcaggcctccaacctcctgggattcccccagtgacaggaagcagctctgggttgctggcacttggtgccctgggaagccaagcccacttggcagtgaaggatgagaagaaccatcatgaactggatcacagagagagagagtccagcacgaacaattctgtgtcaccctcagaaagcctccgggccagtgagaagcaccggggctctgcagactacagcatggaagccaagaagcggaaagcggaagagaaagacagcctgagccgatacgacagcgatggggacaagagtgatgacctggtagtggatgtctccaatgaggacccagcgacaccccgggtgagcccagcacactcccctcctgaaaatgggctggacaaagcccgaggtctgaagaaagacgcccccaccagcccagcctctgtggcttcctccagcagcacgccttcctccaagaccaaagaccttggtcataatgacaaatcttccacacctgggctcaagtccaacacaccaacgccaaggaacgatgccccaactccaggcaccagcaccaccccggggctccggtcaatgccgggcaaacctccaggcatggacccgatagcctcggccctgcgcacacccatctccctcaccagctcctatgcagcaccctttgccatgatgagccaccacgagatgaatggctccctcaccagcccaagcgcctatgctggcctacacaacatcccatcccagatgagcgccgccgcagcagctgcagccgccgcctatggccgatcgccaatggttggttttgaccctcatcccccgatgcgggccacaggcctgccctccagtcttgcctccattcctggagggaaaccggcgtactccttccatgtgagtgccgacgggcagatgcaacctgtgcccttcccccatgatgcgctagcaggccccggcattcccaggcatgcccggcagatcaacacactcagccatggggaggtggtatgtgccgtgaccatcagcaaccccacgaggcacgtctacacaggcggcaagggctgtgtgaagatatgggacatcagccagccgggcagcaagagtcccatctcccagctggactgcctgaacagggacaactacatccgctcatgcaagcttctccctgatgggcgcacgctcattgtgggtggtgaggccagcacactcaccatctgggacctggcctcgcccacgccccgcatcaaggctgagctgacgtcctcggctccagcctgttacgccctggccatcagtcctgatgccaaagtctgtttctcctgctgcagtgatgggaacattgcagtttgggatctgcacaaccagaccctggtcaggcagttccagggccacacggatggggccagctgtatagacatctctcatgatggcactaaactgtggaccgggggcctggacaacactgtgcgctcctgggacctgcgtgaagggcggcagctacagcaacacgacttcacctcccagatcttctccctgggttactgccccactggggagtggctcgccgtgggcatggagagcagcaacgtagaggttctgcaccacaccaagccggacaagtaccaactgcatctgcacgagagctgcgtgctgtccctcaagtttgcctactgtggcaagtggtttgtgagcactgggaaagacaaccttctcaatgcctggaggacgccttacggagccagcatcttccagtctaaagaatcctcgtccgtcttgagctgtgatatttcagcggatgacaaatatattgtaacgggctctggtgacaagaaggccacagtttacgaggtcatctactgaacaaggactctaagaggcctgtcaaactctgggagacacccacgcggccctgacagggagatgctggccaggccccgaggatggcagaagagtgagggacagcattgtggggctgccctccggaaggtggagaggacgcacgcccctttgtggattgctgtgtctgacagacttgaaggactttctcctctccccctcaccagtgctggcagatgcttcaattagatttctctggaggcttctgctttcgtttcccacacagacaccctcatgagtctctgtctcttcctcccctgtctgatctgtcccctccctactccgggttgggggcatgctggagtagacattgttgtttctggggaggggcgcctcgttctgattgtgcagtgcacaaggtttgtgaacaatgtaaataagagactgtggttgctgactggccagtgagggaggaggccccttcccaccagtgctcgccgtgtattttgcctgctgtatttgtaacccactcgcggtggtggcttttctttctttcttttcttaagattttttttaaacagatgttctgtggggaaagtaggcagagatagagagtcggggagtggaatgagaagcttcctggggatgggcaaaccagcctggcccactgggactgaagtatatctccacacgggttttagcaccatcttcagatggaatgataggcagatggacagacaagctgatggacagacagtactgggatgggccagcttgggctattatccacaggaacagcgaggatttcctgtgggtttctctgctccatcttctcttgccttgccctcacacctcctgacctccatctctttctgcccccctccccccgctcgcctctcccactggaccccatctttctctttggtgcacactcaatcattgtgatgaggaatggaagttcaagggaactccctctctgactctcctccgccagcctagaaaggattccatatgggggtgggagggagacaagcgactcagctcagggagctaccagagaaaaacagcaactggtgtgggtgctttttttttttaatttgaataaaaataattagaagtgatatcctttgataagatgccgtctcccctttcctgctctcagccctttctgctccctgaagccggggatatggtgacccccagggtcgtaagtctaagaagatgggcgggggagcgggctgggggctggggcagacggaatgtcatctgcagatatcacccctagggtaccaatgtttgtggtattaggtttgctttgttttgttttggtcttttgttgttttattaaagaaaagctaaagaccgtgtgagccctgctggtgggctttccatggatgtagcatatggaagagagtttttatattgcatttttatggattattttataatgtgggactcagtctgggaagaaagatgagggtgtctaaaacctgcccatctccagtgctccaactgagcggctgccccgaacaggatgtatgttgggacatggaaagaccgtggcaccccgcaccttcctgagaggcatgtcgacatgcctgtctggagtctgtccatctgtctctgggtttcagatgatcactggccaccacttgttaaggctctagccagaagggagggttagggtggaagagagttattcctgaagaaaaaagaaaaattgaaacatcattgtaatgaagctttttatatttttaaaagttac

SEQ ID NO: 44 - DNA, 588 bp, rattus norvegicus
cctaagtgtatcgtgctttactttttgttactgcaacacatttgatggaacacactgtctcctttaaaaaagaaaaaaagaaaaaaaaaaaagccagacccccgttcttgctgtattttgcttgttgagtacaaagatgaagcgatttgccaaaaagcggccaagttgctcccatttccctgtaattacgaaggtttggttgggcgatgtacgctccagccctcatgcacactaactcttcaatctaattgtttgattaaactcgtatttcctccagaaaggtacataaataagtgaactgttgggcaaattaagaatacttaacagcattgcatccgaaaagtagttatgctgattaaattttgtgtccttgaggactttcaggaaattacttttgatcgtcaataacacaattaagtaaactacacacacattagcatgaataattgataaagaaattatgtattaccacgaagcactgatttataaatcatgacgcaacactccaaggactgtgcaaaactggataacattgaaaaagctggcttgcgttctggaagaaaagaaaaaaaaaaaaaaaaaaaaaaa

SEQ ID NO: 45 - DNA, 2106 bp, rattus norvegicus
tggtgtcactacacagggctgcctctaggcctggggaacgcaccccagttccgacacctgctgccctggctggatgatgaaccagtctcagagaatggcacctgttggctcggacaaagaactgagtgacctcctggacttcagtatgatgttcccgctacctgtggccaatggcaagggccggcccgcctccctagctggaacccagttcgcaggctcaggactggaggaccgacccagctcagagtcctggggcaacagtgaacagaacagttcttcctttgaccctagccgggcatacagcgaaggtgcccacttcagtgactcccacagcagcctgccgccttccacgttcctaggagctgggcttggaggcaagggcagtgaacggaatgcctatgccaccttcgggagagacaccagtgttggcaccctgagtcaggctggcttcctaccaggtgagctgggcctcagcagtcccgggccactgtccccatcaggcgtcaagagcagctcccagtattacacctcattccccagcaaccctcggcggagagctgcagatggtggcctggatactcagcccaagaaggtccggaaggttccgcctggtcttccttcctcggtatatccatccagctcaggtgataactacagcagggacgccacagcctacccctccgccaagacccccagcagcgcttacccctcccccttctacgtggcagacggcagcctgcacccctcagcggagctctggagtccccctggccaggtgggctttgggcccatgctaggtgatggctcggcccccctgccgcttgcaccgggcagcagttccgtgagcagtggtgcctttgggggcctccagcagcaggatcgcatgggctaccagctgcatgggtctgaggtcaatggtacgctcccagctgtatccagcttctcagctgcccctggcacttacagtgggacttctggccacacgccacccgtgagcggggccgacagcctcttaggcacccgagggactacagccagcagctcaggggatgcccttgggaaggcactggcctccatctactccccggatcactccagcaataatttctcgcccagcccctcaacgcctgtgggttcaccccagggcctgccagggacatcacagtggccccgggcaggagcgcccagtgccttatcccccaactacgatgcaggtctccatggcctgagtaaaatggaagatcgcttggacgaggccatccacgtcctccgcagtcacgctgtcggcactgcaagcgagctccatggactgctgcctggccacagcacgttgaccacaagctttgcgggccccatgtcactgggcgggcggcatgcaggcctggtcagcggaagccaccctgaggatggcctcacaagtggtgccagtcttttgcataaccatgccagccttcccagccagcccagctccctccctgacctctcacagaggccacccgactcctttagtggactcgggagggcaggcgtgactgcaggcgccagtgagatcaagcgggaggagaaagaggacgaggaggtcacatcagtggcagacgctgaggaagacaagaaggacctgaaggtcccacgcacacgtaccagcccagacgaggacgaggacgaccttctccccccagagcagaaggccgagcgggagaaggagcgccgggtggccaataacgcccgagagcgcctgcgtgtccgcgacatcaatgaggcctttaaggagctcggccgcatgtgccagctgcacctcagcactgagaagccgcagaccaaactgctcatcctgcaccaggccgtggccgtcatcctcagcctggagcagcaggtgcgagaacgcaacctgaaccccaaagcagcctgcttgaagcggcgggaggaggagaaggtatctggcgtggtcggggatccgcagctggcgctgtcagccgcccacccgggcctgggtgaggcccacaatccagctgggcacctgtgagccgtcacagggtcttcgttggaccagggaccaccacatctctgcccggggtgcacccaggatggtcctggatgagacatctc

SEQ ID NO: 46 - DNA, 1248 bp, artificial
  dominant negative
atggactacaaggacgacgatgacaagcatatggctagcatgactggtggacagcaaatgggtcgggatcctgacctggaacaacgtgctgaggaactggcccgtgaaaacgaagagctggaaaaagaggccgaagagctggagcaggaactggcagaactggagaacaagctgatcgccctgggagaagaaaatgccactttaaaagctgagctgctctccctgaaattaaagtttggcttaattagctccacggtgtacgcccaagaaatccagaaactcagtaattccacagctgtgtattttcaggactaccagacatccaaggctgccgtgagctcttacgtggacgagcatgagcctgcgatggtagccggaagttgcatctctgtcatcaagcactctcctcagagctcgctctcggatgtgtctgaggtgtcctctgtggagcacacacaggaaagccccgcacagggaggctgccggagccctgagaacaagttccctgtgatcaagcaggagcccgtggagttggagagctttgccagggagtccagagaggagcggggtgcctattccgcctccatctaccagagctacatgggtagctctttctccacctactcccactcccctcccctcctgcaggtccacggctccaccagcaactccccgagaacctcggaggccgacgagggtgtggtgggcaagtcttctgatggggaagacgagcagcaggtccccaaaggccccatccattctccagtggagctgcagcgggtgcacgccacggtggtgaaggttccggaagtgaacccttccgccttaccgcacaagctccggattaaagccaaggccatgcaggtcaaagtggaagctttggacagtgagttcgaaggcatgcagaaactctcttcaccggcagatgcgctagccaaaagacattttgacctggagaagcatggcgcttcgggtacggcccattcctccctccctcctttctcggtgcaggtgacgaacattcaagattggtccctcagatcggaacactggcatcacaaagaactgggcggcaaaactcagggtaccttcaaaacgggtgtggtggaagtcaaagacagtggctataaggtttccgaggctgagaatttgtatttgaagcagggggtggcaaacttatccgcagaggtggtctcgctcaagagattcatagccacacagcctatctcggcttcggactccaggtaa

SEQ ID NO: 47 - protein, 415 aa, artificial
  dominant negative
MDYKDDDDKHMASMTGGQQMGRDPDLEQRAEELARENEELEKEAEELEQELAELENKLIALGEENATLKAELLSLKLKFGLISSTVYAQEIQKLSNSTAVYFQDYQTSKAAVSSYVDEHEPAMVAGSCISVIKHSPQSSLSDVSEVSSVEHTQESPAQGGCRSPENKFPVIKQEPVELESFARESREERGAYSASIYQSYMGSSFSTYSHSPPLLQVHGSTSNSPRTSEADEGVVGKSSDGEDEQQVPKGPIHSPVELQRVHATVVKVPEVNPSALPHKLRIKAKAMQVKVEALDSEFEGMQKLSSPADALAKRHFDLEKHGASGTAHSSLPPFSVQVTNIQDWSLRSEHWHHKELGGKTQGTFKTGVVEVKDSGYKVSEAENLYLKQGVANLSAEVVSLKRFIATQPISASDSR

SEQ ID NO: 48 - DNA, 561 bp, artificial
  dominant negative
atggactacaaggacgacgatgacaagcatatggctagcatgactggtggacagcaaatgggtcgggatccttctccagtggagctgcagcgggtgcacgccacggtggtgaaggttccggaagtgaacccttccgccttaccgcacaagctccggattaaagccaaggccatgcaggtcaaagtggaagctttggacagtgagttcgaaggcatgcagaaactctcttcaccggcagatgcgctagccaaaagacattttgacctggagaagcatggcgcttcgggtacggcccattcctccctccctcctttctcggtgcaggtgacgaacattcaagattggtccctcagatcggaacactggcatcacaaagaactgggcggcaaaactcagggtaccttcaaaacgggtgtggtggaagtcaaagacagtggctataaggtttccgaggctgagaatttgtatttgaagcagggggtggcaaacttatccgcagaggtggtctcgctcaagagattcatagccacacagcctatctcggcttcggactccaggtaa

SEQ ID NO: 49 - protein, 186 aa, artificial
  dominant negative
MDYKDDDDKHMASMTGGQQMGRDPSPVELQRVHATVVKVPEVNPSALPHKLRIKAKAMQVKVEALDSEFEGMQKLSSPADALAKRHFDLEKHGASGTAHSSLPPFSVQVTNIQDWSLRSEHWHHKELGGKTQGTFKTGVVEVKDSGYKVSEAENLYLKQGVANLSAEVVSLKRFIATQPISASDSR

SEQ ID NO: 50 - DNA, 2573 bp, rattus norvegicus
gacgactggtctgcgccgccggagaacctctgtcgctggggctggtccgcgggctccgaggaaccgctgcttcctgagagcgctccgtgagtgaccgcgacttttcaaagctccggatcgcgcgggaccaaccaacgtgagtgcaagcggtgtcttaaccctgcgctccctggagcgaactggggaggagggcgcaggggggagcactgcggtctggagcgcacgctcctaaacaaactttgttactgaagcggggacgcgcgggtatcccccgcttcccggcgcgctgttgcggccccgaaacttctgcgcacagcccaggctaaccccgcgtgaagtgaccgactgttctatgactgcaaagatggaaacgaccttctacgacgatgccctcaacgcctcgttcctccagtccgagagtggcgcctacggctacagtaaccctaagattctgaagcagagcatgaccttgaacctggccgacccggtgggcaatctgaagccgcacctccgagccaagaactcggaccttctcacgtcgcccgacgtcgggctgctcaagctggcgtcgccggagctggagcgcctgatcatccagtccagcaatgggcacatcaccactacaccgacccccactcagttcttgtgccccaagaacgtgaccgacgagcaggagggcttcgccgaaggcttcgtgcgcgccctagctgaactgcatagccagaatacgctgcccagtgtcacctccgcggcacaacctgtcagtggggcgggcatggtcgctcccgctgtggcctcagtagctggcgctggcggcggcggcggctacagcgccagcctgcacagtgagcctccggtctacgccaacctcagcaacttcaacccgggtgcgctgagcagcggcggtggggcgccctcctatggcgcgaccgggctggcctttccatcgcagccccagcagcagcagcagccgcctcagccgccgcaccacttgccccaacagatcccggtgcagcacccgcggctgcaggcgctgaaggaagagccgcagacggtgccggagatgccgggagagacgccgcccttgtcccccatcgacatggagtctcaggagcggatcaaggcggagaggaagcgcatgagaaaccgcatcgctgcctccaagtgccggaaaaggaagctggagcggatcgcccggctagaggaaaaagtgaaaaccttgaaagcgcaaaactccgagctggcgtccacggccaacatgctcagggaacaggtggcacagcttaaacagaaagtcatgaaccacgttaacagtgggtgccaactcatgctaacgcagcagttgcaaacgttttgaggacagactgtcagggctgaggggcagtggaagaaaaaaataacagagataaacttgagaacttgactggttgcgacagagaaaaaaaagtgtccgagtactgaagccaagggtacacaagatggactgggttgcgacctgacggcgcccccaatgtgctggagtgggaaggacgtggcgcgcctggctttggcgaggagccagagagcagcggcctgttcgcgacgctttgcgaacgggctgtgccgcgaccagaacgatggacttttcgttaacattgaccaagaactgcatggacctaacattcgatctcattcagtattaaggggggtgggtgggggcttacaaactgcaatagagactgtagattgcttctctagtgctccgtaagaacacaaagcagggagggctgggaaggggagggaggcttgtgagtaccaggctagattgcggatgaactcccctggcctgcctctctcaactgtgtatgtacatatatttttttttaatttgatgaaagctgattactgtctataataaacagcttcctgcctttgtaagttatttcatgtttgtttgtttgggtgtcctacccagtgtttgtaaataagagatttgaagcattctgagtttaccatttgtaataaagtatataatttttttatgttttgtttctgaaaatttccagaaaggatatttaagaaaacacaataaaccattgagaagtagcccccaacctctctgctgcattatcgatagataatgagagctaggtgaaatgacagctgagtgtctgtatgctggggtgagagcctgtagtccctcccgtctggttgtaggaatagacaccctgcatgctatcattgactcatactctctccccagcaacccacaagtccagactgttgaccagaagatggtgcagtgtttcttaaggctggaagaagagggtgttgcaaggggagagggacagcccactgggaaagcagatgcttgagttgagagccatatcaagtagcatgcgctgtgatcgtttataataataataaagacttcagcaattagatgtttatctcaaagcagggacccatggaaggttttacaaaaggtgtctccttccaacttggattctgacgactcctagaaaaagatgacctttgcttgtgcatatttataatagttcgtatcacaataaatgtattcaaataatggtttt

SEQ ID NO: 51 - DNA, 4457 bp, rattus norvegicus
gggagcttggggttccgacgtcgcggcggagggaacgagccctaaccggatcgctgaggtacaatcccgctcggtgtcgcctgaccgcgtcggctaggagaggccaggcggcccgcgggagcccagcagttcgcgcctggagccagcatagggcggccagtcgggcttcagccccagagacagtggtcacccctgattgcggcaggatggctcagtggaaccagctccagcagctggacacgcgctacctggagcagcttcatcagctgtacagcgatagcttccccatggagctgcggcagttcctggcgccttggattgagagccaagattgggcatatgcagccagcaaagagtcacacgccactctggtgtttcataacctcttgggcgagatcgaccagcagtatagccgattcctgcaggagtccaatgtcctctatcagcacaacctgcgaagaatcaagcagttcctgcagagcaggtatcttgagaagccaatggaaattgcccggattgtggcccgatgcctgtgggaagagtctcgcctcctccagacggcagccacggcagcccagcaagggggccaggccaaccaccccacagctgccgtagtgacggagaagcagcagatgctggaacagcatcttcaggatgtccggaagcgtgtgcaggatctagaacagaaaatgaaagtggtggagaatctccaggatgactttgatttcaactataaaaccctcaagagtcaaggagacatgcaggatctgaatggaaacaaccagtctgtgaccagacagaagatgcagcagctggagcagatgctcacggccctggaccagatgcggaggagcatcgtgagcgagctggcagggctcttgtcagcaatggagtacgtgcagaagacactgaccgatgaagagctggctgactggaagaggcggcagcagatagcgtgcatcggaggccctcccaacatctgcctggaccgtctggaaaactggataacttcattagcagaatctcaacttcagacccgccaacaaattaagaaactggaggagctgcagcagaaagtgtcctacaagggggaccctattgtgcagcaccggccaatgctggaggagaggatcgtggatctgttcagaaacttaatgaagagtgccttcgtggtggagcggcagccctgtatgcccatgcacccggaccggcccttagtcatcaagactggtgtccagtttaccacaaaagtcaggttgctggtcaaatttcctgagttgaattatcagcttaaaattaaagtgtgcattgataaggactctggggatgttgctgccctcagagggtctcggaaatttaacattctgggcacgaacacaaaggtgatgaacatggaggagtccaacaacggcagcctgtctgcagagttcaagcacctgaccctgagggagcagagatgtgggaatgggggccgtgccaattgtgatgcctccttgattgtcactgaggagctgcacctgatcacctttgagacagaggtgtaccaccaaggtctcaagatcgacctagagacccactccttgccagtcgtggtgatctccaacatctgtcagatgcctaatgcttgggcatcaatcctgtggtataacatgctgaccaataaccccaagaacgtgaacttcttcactaagcctccgattggaacctgggaccaagtggccgaggtgctgagctggcagttctcgtccaccaccaagcgagggctgagcatcgagcagctgaccacgctggccgagaagctcttagggcctggtgtgaactactcagggtgtcagatcacatgggctaagttttgcaaagaaaacatggccggcaagggcttctcgttctgggtctggctagacaatatcatcgaccttgtgaaaaagtatatcttggccctttggaatgaagggtacatcatgggtttcatcagcaaggagcgggagagggccatcctaagcacaaagcccccgggcaccttcctgctgcggttcagtgagagcagcaaggaaggaggggtcactttcacttgggtggaaaaggacatcagtggcaagacccagatccagtctgtagaaccatataccaagcagcagctgaacaacatgtcatttgctgaaatcatcatgggctataagatcatggacgctaccaacatcctggtatccccactggtctacctctaccctgacattcccaaggaggaggcattcggaaagtattgtcgccccgagagccaggagcaccctgaagctgacccaggtagtgctgccccttacctgaagaccaagttcatctgtgtgacaccaacgacctgcagcaataccattgacctgccgatgtccccccgcactttagattcattgatgcagtttggaaataacggggaaggcgctgagccctcagcaggagggcagtttgagtcgctcacgtttgacatggatctgacctcggagtgtgctacctccccgatgtgaggagctgaaagcggaaactgcagagtatgactgagacacctgccccatgttccaccccttaagcagccaaactcccagatcatctgaaactcccaactttgtggttccagattttttctttaatctcctacttctgctatctttgggcaatctgggtactttttaaaatagagaactgagtgagcgtgggtgatgcgctcttatgtgaggaggagggcacctcttgagtccgtgatggctgtgaaagcagagggctgggggggagcgaaaggaaacacctatgttctgtccgcctgtcctcctggtttgttgttggacaagcgcctcctggtgcccatggcatcctgctgcccctttctgtgagctgataccccaggctggggactcccggctctgcactttcaacctggttaatgtccacatagaagctaggactaagcctataggcttctctttaaattaaaaaaaaaaatactaagaattaaagggcaaaacacactgaaacagcatagcctttccatatcaaagaaaactcggttaacagccttttcggtgctttaagctgtctttaggctgatcatttatataaactctgcaatggtttcaaatcaaatctgtaaaagacatctgagaactgtggttcaaacacaaagattagagagaacctagatatcccagactcttgtcatgaactgggttttgtttacccagtatgcttgtctgttggaggggtgaggtcggccaagggcactggaaaacctttgtcatacccccctccccagcccagactctggaccctgttccagggtcatcctgccctgtgggtgccttactgggcctagggtcaacctggcttcctttcccacttgaccttgctagtggtatgtctccttcccatgtccaaaggcccctgtcctgcttacattgggaatccctgtctcagaaccttgtgtcgagagggattgccttagaggtttgaacctaactcagactacaggtcctcagcaaagctcagggagtatggtccttattctatgtgcttggttcccaggggtacctgtaaccacaggacaaaagctgactgataaaatccaggtctgcccttcatgtgagtggcgtactcccgccccccccccccccgccccgagagtgccgattgtatgctccccgttggggctcctaggtgaggtgggacagagtgcctgcccctactgtggccttcctgtcacctgcttgcctcagtcactgagcatacttgaacactgagtggttcaaggcaagcctcccctgatacaggggcatggctagattcagtgactcaaagccaccttactcagctgatctgtctgtggaattgtttttctccagttaaccagtgtctgaattaagggcagtgaggacattgtctccaagacaaactgcttgccttgaccaccccagccttctgcttcgaggcagtcattgctctcccttccctggccaggttctttagttacacaataagctgaactcataaactgaaagggtatttaggaaggcaaggcgtgggcatgttgatggcttccaatcctggggacccaggaacaaggtgagggcttctctggggcaggtgttgtacctcaggggttctgggaagtctgtatctgggttaatcacccatagtgagcccttggaacggcccacttcccctccccttggccccacttgtccccaacctcacccagccatgcctgcagtttgcttggcctcccagtacagtggcttttattctgtcctggtcactgcactcaaattccaatgtatactttctagtgtaaaaatttatattattgtgggttgttttatgttgttgtttgtttttgtatattgctgtaactactttaacatccagaaataaagattatataggaaaaaaaaaaaaaaaaaaaaaaaaaaaaa

SEQ ID NO: 52 - DNA, 4978 bp, homo sapiens
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 53 - DNA, 3006 bp, homo sapiens
agtcggcggcggctgctgctgcctgtggcccgggcggctgggagaagcggagtgttggtgagtgacgcggcggaggtgtagtttgacgcggtgtgttacgtgggggagagaataaaactccagcgagatccgggccgtgaacgaaagcagtgacggaggagcttgtaccaccggtaactaaatgaccatggaatctggagccgagaaccagcagagtggagatgcagctgtaacagaagctgaaaaccaacaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagtcttcctgtaaggacttaaaaagacttttctccggaacacagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacctgccatcaccactgtaacggtgccaactccaatttaccaaactagcagtggacagtatattgccattacccagggaggagcaatacagctggctaacaatggtaccgatggggtacagggcctgcaaacattaaccatgaccaatgcagcagccactcagccgggtactaccattctacagtatgcacagaccactgatggacagcagatcttagtgcccagcaaccaagttgttgttcaagctgcctctggagacgtacaaacataccagattcgcacagcacccactagcactattgcccctggagttgttatggcatcctccccagcacttcctacacagcctgctgaagaagcagcacgaaagagagaggtccgtctaatgaagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatcagattaatttgggatttaaattttcacctgttaaggtggaaaatggactggcttggccacaacctgaaagacaaaataaacattttattttctaaacatttctttttttctatgcgcaaaactgcctgaaagcaactacagaatttcattcatttgtgcttttgcattaaactgtgaatgttccaacacctgcctccacttctcccctcaagaaattttcaacgccaggaatcatgaagagacttctgcttttcaacccccaccctcctcaagaagtaataatttgtttacttgtaaattgatgggagaaatgaggaaaagaaaatctttttaaaaatgatttcaaggtttgtgctgagctccttgattgccttagggacagaattaccccagcctcttgagctgaagtaatgtgtgggccgcatgcataaagtaagtaaggtgcaatgaagaagtgttgattgccaaattgacatgttgtcacattctcattgtgaattatgtaaagttgttaagagacataccctctaaaaaagaactttagcatggtattgaaggaattagaaatgaatttggagtgctttttatgtatgttgtcttcttcaatactgaaaatttgtccttggttcttaaaagcattctgtactaatacagctcttccatagggcagttgttgcttcttaattcagttctgtatgtgttcaacatttttgaatacattaaaagaagtaaccaactgaacgacaaagcatggtatttgaattttaaattaaagcaaagtaaataaaagtacaaagcatattttagttagtactaaattcttagtaaaatgctgatcagtaaaccaatcccttgagttatataacaagatttttaaataaatgttattgtcctcaccttcaaaaatatttatattgtcactcatttacgtaaaaagatatttctaatttactgttgcccattgcacttacataccaccaccaagaaagccttcaagatgtcaaataaagcaaagtgatatatatttgtttatgaaatgttacatgtagaaaaatactgattttaaatattttccatattaacaatttaacagagaatctctagtgaattttttaaatgaaagaagttgtaaggatataaaaagtacagtgttagatgtgcacaaggaaagttattttcagacatatttgaatgactgctgtactgcaatatttggattgtcattcttacaaaacatttttttgttctcttgtaaaaagagtagttattagttctgctttagctttccaatatgctgtatagcctttgtcattttataattttaattcctgattaaaacagtctgtatttgtgtatatcatacattgttttcaataccacttttaattgttactcattttattcactaagctcgataaatctaacagttactcttaaaaaaaaaaaaaagactaaggtggattttaaaaattggaaactgacataatgttaggttataatttctcatttggagccgggcgcagtggctcacgcctgtaatcccagcactttgggaggccaaggtgggtggatcacctgtggtcaagagttcaagaccagcctggccatcatggtgaaaccccatctctactaaaaatacaaaaattagccaggcgtggtggctggcgcctgtaatcccagctactcaggaggttgaggcagcagaattgcttgaacccaggaggcagagggttgcagtgagccgagatagcaccattgcactccagcctgggcgactccatctcaaaaaataaaaaaaaaaaaaaa

SEQ ID NO: 54 - DNA, 4396 bp, homo sapiens
gcctgaggtgcccgccctggccccaggagaatgaaccagccgcagaggatggcgcctgtgggcacagacaaggagctcagtgacctcctggacttcagcatgatgttcccgctgcctgtcaccaacgggaagggccggcccgcctccctggccggggcgcagttcggaggttcaggtcttgaggaccggcccagctcaggctcctggggcagcggcgaccagagcagctcctcctttgaccccagccggaccttcagcgagggcacccacttcactgagtcgcacagcagcctctcttcatccacattcctgggaccgggactcggaggcaagagcggtgagcggggcgcctatgcctccttcgggagagacgcaggcgtgggcggcctgactcaggctggcttcctgtcaggcgagctggccctcaacagccccgggcccctgtccccttcgggcatgaaggggacctcccagtactacccctcctactccggcagctcccggcggagagcggcagacggcagcctagacacgcagcccaagaaggtccggaaggtcccgccgggtcttccatcctcggtgtacccacccagctcaggtgaggactacggcagggatgccaccgcctacccgtccgccaagacccccagcagcacctatcccgcccccttctacgtggcagatggcagcctgcacccctcagccgagctctggagtcccccgggccaggcgggcttcgggcccatgctgggtgggggctcatccccgctgcccctcccgcccggtagcggcccggtgggcagcagtggaagcagcagcacgtttggtggcctgcaccagcacgagcgtatgggctaccagctgcatggagcagaggtgaacggtgggctcccatctgcatcctccttctcctcagcccccggagccacgtacggcggcgtctccagccacacgccgcctgtcagcggggccgacagcctcctgggctcccgagggaccacagctggcagctccggggatgccctcggcaaagcactggcctcgatctactccccggatcactcaagcaataacttctcgtccagcccttctacccccgtgggctccccccagggcctggcaggaacgtcacagtggcctcgagcaggagcccccggtgccttatcgcccagctacgacgggggtctccacggcctgcagagtaagatagaagaccacctggacgaggccatccacgtgctccgcagccacgccgtgggcacagccggcgacatgcacacgctgctgcctggccacggggcgctggcctcaggtttcaccggccccatgtcgctgggtgggcggcacgcaggcctggttggaggcagccaccccgaggacggcctcgcaggcagcaccagcctcatgcacaaccacgcggccctccccagccagccaggcaccctccctgacctgtctcggcctcccgactcctacagtgggctagggcgagcaggtgccacggcggccgccagcgagatcaagcgggaggagaaggaggacgaggagaacacgtcagcggctgaccactcggaggaggagaagaaggagctgaaggccccccgggcccggaccagcccagacgaggacgaggacgaccttctccccccagagcagaaggccgagcgggagaaggagcgccgggtggccaataacgcccgggagcggctgcgggtccgtgacatcaacgaggcctttaaggagctggggcgcatgtgccaactgcacctcaacagcgagaagccccagaccaaactgctcatcctgcaccaggctgtctcggtcatcctgaacttggagcagcaagtgcgagagcggaacctgaatcccaaagcagcctgtttgaaacggcgagaagaggaaaaggtgtcaggtgtggttggagacccccagatggtgctttcagctccccacccaggcctgagcgaagcccacaaccccgccgggcacatgtgaaaggtatgcctccgtgggacgagccacccgctttcagccctgtgctctggccccagaagccggactcgagaccccgggcttcatccacatccacacctcacacacctgttgtcagcatcgagccaacaccaacctgacaaggttcggagtgatgggggcggccaaggtgacactgggtccaggagctccctggggccctggcctaccactcactggcctcgctccccctgtccccgaatctcagccaccgtgtcactctgtgacctgtcccatggatcctgaaactgcatcttggccctgttgcctgggctgacaggagcattttttttttttccagtaaacaaaacctgaaagcaagcaacaaaacatacactttgtcagagaagaaaaaaatgccttaactataaaaagcggagaaatggaaacatatcactcaagggggatgctgtggaaacctggcttattcttctaaagccaccagcaaattgtgcctaagcgaaatattttttttaaggaaaataaaaacattagttacaagattttttttttcttaaggtagatgaaaattagcaaggatgctgcctttggtctctggtttttttaagctttttttgcatatgttttgtaagcaacaaatttttttgtataaaagtcccgtgtctctcgctatttctgctgctgttcctagactgagcattgcatttcttgatcaaccagatgattaaacgttgtattaaaaagaccccgtgtaaacctgagccccccccgtccccccccccggaagccactgcacacagacagacggggacaggcggcgggtcttttgtttttttgatgttgggggttctcttggttttgtcatgtggaaagtgatgcgtgggcgttccctgatgaaggcaccttggggcttccctgccgcatcctctcccctcaggaaggggactgacctgggcttgggggaagggacgtcagcaaggtggctctgaccctcccaggtgactctgccaagcagctgtggccccagcggtaccctacacaacgccctccccaggcccccctaagctgctctcccttggaacctgcacagctctctgaaatggggcattttgttgggaccagtgacccctggcatggggaccacaccctggagcccggtgctggggacctcctggacaccctgtccttcactccttgccccagggacccaggctcatgctctgaactctggctgagaggagtctgctcaggagccagcacaggacaccccccaccccaccccaccatgtccccattacaccagagggccatcgtgacgtagacaggatgccaggggcctgaccagcctccccaatgctggggagcatccctggcctggggccacacctgctgccctccctctgtgtggtccaagggcaagagtggctggagccgggggactgtgctggtctgagccccacgaaggccttgggctgtggctccgaccctgctgcagaaccagcagggtgtcccctcgggcccatctgtgtcccatgtcccagcacccaggcctctctccaggtctccttttctggtcttttgccatgagggtaaccagctcttcccagctggctgggactgtcttgggtttaaaactgcaagtctcctaccctgggatcccatccagttccacacgaactagggcagtggtcactgtggcacccaggtgtgggcctggctagctgggggccttcatgtgcccttcatgcccctccctgcattgaggccttgtggacccctgggctggctgtgttcatccccgctgcaggtcgggcgtctccccccgtgccactcctgagactccaccgttacccccaggagatcctggactgcctgactcccctccccagactggcttgggagcctgggccccatggtagatgcaagggaaacctcaaggccagctcaatgcctggtatctgcccccagtccaggccaggcggaggggaggggctgtccggctgcctctcccttctcggtggcttcccctgcgccctgggagtttgatctcttaagggaacttgcctctccctcttgttttgctcctgccctgcccctaggtctgggtggcagtggccccatagcctctggaactgtgcgttctgcatagaattcaaacgagattcacccagcgcgaggaggaagaaacagcagttcctgggaaccacaattatggggggtggggggtgtgatctgagtgcctcaagatggttttcaaaaaattttttttaaagaaaataattgtatacgtgtcaacacagctggctggatgattgggactttaaaacgaccctctttcaggtggattcagagacctgtcctgtatataacagcactgtagcaataaacgtgacattttataaag

SEQ ID NO: 55 - DNA, 2357 bp, homo sapiens
gaatcacgacccctccctgccatgtatccgcagggcagacatccggctccccatcaacccgggcagccgggatttaaattcacggtggctgagtcttgtgacaggatcaaagacgaattccagttcctgcaagctcagtatcacagcctcaaagtggagtacgacaagctggcaaacgagaagacggagatgcagcgccattatgtgatgtactatgagatgtcctatggcttgaacattgaaatgcacaagcagacagagattgcgaagagactgaacacaattttagcacagatcatgcctttcctgtcacaagagcaccagcagcaggtggcgcaggcagtggagcgcgccaagcaggtcaccatgacggagctgaacgccatcatcgggcagcagcagctccaggcgcagcacctctcccatgccacacacggccccccggtccagttgccaccccacccgtcaggtctccagcctccaggaatccccccagtgacagggagcagctccgggctgctggcactgggcgccctgggcagccaggcccatctgacggtgaaggatgagaagaaccaccatgaactcgatcacagagagagagaatccagtgcgaataactctgtgtcaccctcggaaagcctccgggccagtgagaagcaccggggctctgcggactacagcatggaagccaagaagcggaaggtggaggagaaggacagcttgagccgatacgacagtgatggagacaagagtgatgatctggtggtggatgtttccaatgaggaccccgcaacgccccgggtcagcccggcacactcccctcctgaaaatgggctggacaaggcccgtagcctgaaaaaagatgcccccaccagccctgcctcggtggcctcttccagtagcacaccttcctccaagaccaaagaccttggtcataacgacaaatcctccacccctgggctcaagtccaacacaccaaccccaaggaacgacgccccaactccaggcaccagcacgaccccagggctcaggtcgatgccgggtaaacctccgggcatggacccgataggtataatggcctcggctctgcgcacgcccatctccatcaccagctcctatgcggcgcccttcgccatgatgagccaccatgagatgaacggctccctcaccagtcctggcgcctacgccggcctccacaacatcccaccccagatgagcgccgccgccgctgctgcagccgctgcctatggccgatcgccaatggtgagctttggagctgttggttttgaccctcaccccccgatgcgggccacaggcctcccctcaagcctggcctccattcctggaggaaaaccagcgtactcattccatgtgagtgctgatgggcagatgcagcccgtgcccttcccccacgacgccctggcaggccccggcatcccgaggcacgcccggcagatcaacacactcagccacgggggggtggtgtgtgccgtgaccatcagcaaccccagcaggcacgtctacacaggtggcaagggctgcgtgaagatctgggacatcagccagccaggcagcaagagccccatctcccagctggactgcctgaacagggacaattacatgcgctcctgcaagctgcaccctgatgggcgcacgctcatcgtgggcggcgagggcagcacgctcaccatctgggacctggcctcgcccacgccccgcatcaaggccgagctgacgtcctcggctcccgcctgttatgccctggccattagccctgacgccaaagtctgcttctcctgctgcagcgatgggaacattgctgtctgggacctgcacaaccagaccctggtcaggcagttccagggccacacagatggggccagctgcatagacatctcccatgatggcaccaaactgtggacagggggcctggacaacacggtgcgctcctgggacctgcgggagggccgacagctacagcagcatgacttcacttcccagatcttctcgctgggctactgccccactggggagtggctggctgtgggcatggagagcagcaacgtggaggtgctgcaccacaccaagcctcacaagtaccagctgcacctgcacgagagctgcgtgctctccctcaagttcgcctactgcggcaagtggttcgtgagcactgggaaagataaccttctcaacgcctggaggacgccttatggagccagcatatcccagtctaaagaatcctcgtctgtcttgagttgtgacatttcagcggatgacaaatacattgtaacaggctctggtgacaagaaggccacagtttatgaggtcatctactaaacaagaactccagcagg

SEQ ID NO: 56 - DNA, 3338 bp, homo sapiens
gacatcatgggctatttttaggggttgactggtagcagataagtgttgagctcgggctggataagggctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtgtttcgggagtgtccagagagcctggtctccagccgcccccgggaggagagccctgctgcccaggcgctgttgacagcggcggaaagcagcggtacccacgcgcccgccgggggaagtcggcgagcggctgcagcagcaaagaactttcccggctgggaggaccggagacaagtggcagagtcccggagcgaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcccgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccagagccctgttgcggccccgaaacttgtgcgcgcagcccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttgcgtcctgacggcgcccccagtgtgcacgagtgggaaggacttggcgcgccctcccttggcgtggagccagggagcggccgcctgcgggctgccccgctttgcggacgggctgtccccgcgcgaacggaacgttggacttttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgccttcgttaactgtgtatgtacatatatatattttttaatttgatgaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttagaaataaatgtacctgatgtacctgatgctatggtcaggttatactcctcctcccccagctatctatatggaattgcttaccaaaggatagtgcgatgtttcaggaggctggaggaaggggggttgcagtggagagggacagcccactgagaagtcaaacatttcaaagtttggattgtatcaagtggcatgtgctgtgaccatttataatgttagtagaaattttacaataggtgcttattctcaaagcaggaattggtggcagattttacaaaagatgtatccttccaatttggaatcttctctttgacaattcctagataaaaagatggcctttgcttatgaatatttataacagcattcttgtcacaataaatgtattcaaataccaaaaaaaaaaaaaaaaa

SEQ ID NO: 57 - protein, 341 aa, homo sapiens
MTMESGAENQQSGDAAVTEAENQQMTVQAQPQIATLAQVSMPAAHATSSAPTVTLVQLPNGQTVQVHGVIQAAQPSVIQSPQVQTVQSSCKDLKRLFSGTQISTIAESEDSQESVDSVTDSQKRREILSRRPSYRKILNDLSSDAPGVPRIEEEKSEEETSAPAITTVTVPTPIYQTSSGQYIAITQGGAIQLANNGTDGVQGLQTLTMTNAAATQPGTTILQYAQTTDGQQILVPSNQVVVQAASGDVQTYQIRTAPTSTIAPGVVMASSPALPTQPAEEAARKREVRLMKNREAARECRRKKKEYVKCLENRVAVLENQNKTLIEELKALKDLYCHKSD

SEQ ID NO: 58 - DNA, 807 bp, homo sapiens
atggtgctgatgagctgtgaccaacggttgatcagtttgctcttggtaggcactgcaacctttggcaatcactcttcgggggattttgatgacgggtttctgcgtagaaaacagcgccggaaccggacgacgttcactcttcagcagctggaagctctcgaggccgtttttgcccaaacacactatccagatgtcttcaccagagaagagctcgccatgaaaataaacctcacagaagccagagtgcaggtttggttccagaacagaagggccaaatggaggaagacagagagaggggcctcagaccaggagccaggagccaaggagcccatggcagaggtgacacctcctccagtgagaaacatcaactccccgccccctggggaccaagcccggagtaagaaggaggcgctggaggcccagcagagcctggggcgaacggtaggtcctgcagggcctttcttcccctcctgcttgccggggactctcctgaacacggccacctacgcccaggccttgtcccatgtggcttccctcaaagggggcccactgtgctcttgctgcgtcccagaccccatgggactctccttccttcccacctatggctgccagagtaaccgcacggccagcgtggccaccctgcgcatgaaggcccgcgagcactcagaagctgtcctgcagtcagccaacctcctgccttccaccagcagcagccccggccctgtcgccaagccggcgcccccagatggcagccaggaaaagacctctcccaccaaggaacagagcgaggcagagaagagtgtatga

SEQ ID NO: 59 - DNA, 4298 bp, homo sapiens
cagacaggatattcactgctgtggcaaggcctgtagagagtttcgaagttaggaggactcaagacggtccctccctggacttttctgaaggggctcaaaagatgacacgcgccagagctggaaggcgtcgccaattggtccaacttttccctcctccctttttgcggatgagaaaaactgaggcccaggtttgggatttccagagcccgggatttcccggcaacgccgacaaccacattcccccggctattctgacccgccccggttccgggacgctccctgggagccgccgccgagggcctgctgggactcccggggaccccgccgtcggggcagcccccacgcccggcgccgcccgccggaacggcgccgctgttgcgcacttgcaggggagccggcgactgagggcgaggcagggagggagcaagcggggctgggagggctgctggcgcgggctcgccggctgtgtatggtctatcgcaggcagctgacctttgaggaggaaatcgctgctctccgctccttcctgtagtaacagccgccgctgccgccgccgccaggaaccccggccgggagcgagagccgcggggcgcagagccggcccggctgccggacggtgcggccccaccaggtgaacggccatggcgggctggatccaggcccagcagctgcagggagacgcgctgcgccagatgcaggtgctgtacggccagcacttccccatcgaggtccggcactacttggcccagtggattgagagccagccatgggatgccattgacttggacaatccccaggacagagcccaagccacccagctcctggagggcctggtgcaggagctgcagaagaaggcggagcaccaggtgggggaagatgggtttttactgaagatcaagctggggcactacgccacgcagctccagaaaacatatgaccgctgccccctggagctggtccgctgcatccggcacattctgtacaatgaacagaggctggtccgagaagccaacaattgcagctctccggctgggatcctggttgacgccatgtcccagaagcaccttcagatcaaccagacatttgaggagctgcgactggtcacgcaggacacagagaatgagctgaagaaactgcagcagactcaggagtacttcatcatccagtaccaggagagcctgaggatccaagctcagtttgcccagctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacattcatcattgagaagcagcctcctcaggtcctgaagacccagaccaagtttgcagccaccgtacgcctgctggtgggcgggaagctgaacgtgcacatgaatcccccccaggtgaaggccaccatcatcagtgagcagcaggccaagtctctgcttaaaaatgagaacacccgcaacgagtgcagtggtgagatcctgaacaactgctgcgtgatggagtaccaccaagccacgggcaccctcagtgcccacttcaggaacatgtcactgaagaggatcaagcgtgctgaccggcggggtgcagagtccgtgacagaggagaagttcacagtcctgtttgagtctcagttcagtgttggcagcaatgagcttgtgttccaggtgaagactctgtccctacctgtggttgtcatcgtccacggcagccaggaccacaatgccacggctactgtgctgtgggacaatgcctttgctgagccgggcagggtgccatttgccgtgcctgacaaagtgctgtggccgcagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtccgtgtcctggtcccagttcaacagggagaacttgccgggctggaactacaccttctggcagtggtttgacggggtgatggaggtgttgaagaagcaccacaagccccactggaatgatggggccatcctaggttttgtgaataagcaacaggcccacgacctgctcatcaacaagcccgacgggaccttcttgttgcgctttagtgactcagaaatcgggggcatcaccatcgcctggaagtttgactccccggaacgcaacctgtggaacctgaaaccattcaccacgcgggatttctccatcaggtccctggctgaccggctgggggacctgagctatctcatctatgtgtttcctgaccgccccaaggatgaggtcttctccaagtactacactcctgtgctggctaaagctgttgatggatatgtgaaaccacagatcaagcaagtggtccctgagtttgtgaatgcatctgcagatgctgggggcagcagcgccacgtacatggaccaggccccctccccagctgtgtgcccccaggctccctataacatgtacccacagaaccctgaccatgtactcgatcaggatggagaattcgacctggatgagaccatggatgtggccaggcacgtggaggaactcttacgccgaccaatggacagtcttgactcccgcctctcgccccctgccggtcttttcacctctgccagaggctccctctcatgaatgtttgaatcccacgcttctctttggaaacaatatgcaatgtgaagcggtcgtgttgtgagtttagtaaggttgtgtacactgacacctttgcaggcatgcatgtgcttgtgtgtgtgtgtgtgtgtgtgtccttgtgcatgagctacgcctgcctcccctgtgcagtcctgggatgtggctgcagcagcggtggcctcttttcagatcatggcatccaagagtgcgccgagtctgtctctgtcatggtagagaccgagcctctgtcactgcaggcactcaatgcagccagacctattcctcctgggcccctcatctgctcagcagctatttgaatgagatgattcagaaggggaggggagacaggtaacgtctgtaagctgaagtttcactccggagtgagaagctttgccctcctaagagagagagacagagagacagagagagagaaagagagagtgtgtgggtctatgtaaatgcatctgtcctcatgtgttgatgtaaccgattcatctctcagaagggaggctgggggttcattttcgagtagtattttatactttagtgaacgtggactccagactctctgtgaaccctatgagagcgcgtctgggcccggccatgtccttagcacaggggggccgccggtttgagtgagggtttctgagctgctctgaattagtccttgcttggctgcttggccttgggcttcattcaagtctatgatgctgttgcccacgtttcccgggatatatattctctcccctccgttgggccccagccttctttgcttgcctctctgtttgtaaccttgtcgacaaagaggtagaaaagattgggtctaggatatggtgggtggacaggggccccgggacttggagggttggtcctcttgcctcctggaaaaaacaaaaacaaaaaactgcagtgaaagacaagctgcaaatcagccatgtgctgcgtgcctgtggaatctggagtgaggggtaaaagctgatctggtttgactccgctggaggtggggcctggagcaggccttgcgctgttgcgtaactggctgtgttctggtgaggccttgctcccaaccccacacgctcctccctctgaggctgtaggactcgcagtcaggggcagctgaccatggaagattgagagcccaaggtttaaacttctctgaagggaggtggggatgagaagaggggtttttttgtactttgtacaaagaccacacatttgtgtaaacagtgttttggaataaaatatttttttcat

SEQ ID NO: 60 - DNA, 3136 bp, homo sapiens
gcgcagaacttggggagccgccgccgccatccgccgccgcagccagcttccgccgccgcaggaccggcccctgccccagcctccgcagccgcggcgcgtccacgcccgcccgcgcccagggcgagtcggggtcgccgcctgcacgcttctcagtgttccccgcgccccgcatgtaacccggccaggcccccgcaactgtgtcccctgcagctccagccccgggctgcacccccccgccccgacaccagctctccagcctgctcgtccaggatggccgcggccaaggccgagatgcagctgatgtccccgctgcagatctctgacccgttcggatcctttcctcactcgcccaccatggacaactaccctaagctggaggagatgatgctgctgagcaacggggctccccagttcctcggcgccgccggggccccagagggcagcggcagcaacagcagcagcagcagcagcgggggcggtggaggcggcgggggcggcagcaacagcagcagcagcagcagcaccttcaaccctcaggcggacacgggcgagcagccctacgagcacctgaccgcagagtcttttcctgacatctctctgaacaacgagaaggtgctggtggagaccagttaccccagccaaaccactcgactgccccccatcacctatactggccgcttttccctggagcctgcacccaacagtggcaacaccttgtggcccgagcccctcttcagcttggtcagtggcctagtgagcatgaccaacccaccggcctcctcgtcctcagcaccatctccagcggcctcctccgcctccgcctcccagagcccacccctgagctgcgcagtgccatccaacgacagcagtcccatttactcagcggcacccaccttccccacgccgaacactgacattttccctgagccacaaagccaggccttcccgggctcggcagggacagcgctccagtacccgcctcctgcctaccctgccgccaagggtggcttccaggttcccatgatccccgactacctgtttccacagcagcagggggatctgggcctgggcaccccagaccagaagcccttccagggcctggagagccgcacccagcagccttcgctaacccctctgtctactattaaggcctttgccactcagtcgggctcccaggacctgaaggccctcaataccagctaccagtcccagctcatcaaacccagccgcatgcgcaagtaccccaaccggcccagcaagacgcccccccacgaacgcccttacgcttgcccagtggagtcctgtgatcgccgcttctcccgctccgacgagctcacccgccacatccgcatccacacaggccagaagcccttccagtgccgcatctgcatgcgcaacttcagccgcagcgaccacctcaccacccacatccgcacccacacaggcgaaaagcccttcgcctgcgacatctgtggaagaaagtttgccaggagcgatgaacgcaagaggcataccaagatccacttgcggcagaaggacaagaaagcagacaaaagtgttgtggcctcttcggccacctcctctctctcttcctacccgtccccggttgctacctcttacccgtccccggttactacctcttatccatccccggccaccacctcatacccatcccctgtgcccacctccttctcctctcccggctcctcgacctacccatcccctgtgcacagtggcttcccctccccgtcggtggccaccacgtactcctctgttccccctgctttcccggcccaggtcagcagcttcccttcctcagctgtcaccaactccttcagcgcctccacagggctttcggacatgacagcaaccttttctcccaggacaattgaaatttgctaaagggaaaggggaaagaaagggaaaagggagaaaaagaaacacaagagacttaaaggacaggaggaggagatggccataggagaggagggttcctcttaggtcagatggaggttctcagagccaagtcctccctctctactggagtggaaggtctattggccaacaatcctttctgcccacttccccttccccaattactattccctttgacttcagctgcctgaaacagccatgtccaagttcttcacctctatccaaagaacttgatttgcatggattttggataaatcatttcagtatcatctccatcatatgcctgaccccttgctcccttcaatgctagaaaatcgagttggcaaaatggggtttgggcccctcagagccctgccctgcacccttgtacagtgtctgtgccatggatttcgtttttcttggggtactcttgatgtgaagataatttgcatattctattgtattatttggagttaggtcctcacttgggggaaaaaaaaaaaagaaaagccaagcaaaccaatggtgatcctctattttgtgatgatgctgtgacaataagtttgaacctttttttttgaaacagcagtcccagtattctcagagcatgtgtcagagtgttgttccgttaacctttttgtaaatactgcttgaccgtactctcacatgtggcaaaatatggtttggtttttctttttttttttttttgaaagtgttttttcttcgtccttttggtttaaaaagtttcacgtcttggtgccttttgtgtgatgcgccttgctgatggcttgacatgtgcaattgtgagggacatgctcacctctagccttaaggggggcagggagtgatgatttgggggaggctttgggagcaaaataaggaagagggctgagctgagcttcggttctccagaatgtaagaaaacaaaatctaaaacaaaatctgaactctcaaaagtctatttttttaactgaaaatgtaaatttataaatatattcaggagttggaatgttgtagttacctactgagtaggcggcgatttttgtatgttatgaacatgcagttcattattttgtggttctattttactttgtacttgtgtttgcttaaacaaagtgactgtttggcttataaacacattgaatgcgctttattgcccatgggatatgtggtgtatatccttccaaaaaattaaaacgaaaataaagtagctgcgattggg

SEQ ID NO: 61 - DNA, 1125 bp, rattus norvegicus
gaaagcagtgactgaggagcttgtaccaccggtgactagatgaccatggactctggagcagacaaccagcagagtggagatgctgctgtaacagaagctgaaagtcaacaaatgacagttcaagcccagccacagattgccacattagcccaggtatccatgccagcagctcatgcgacgtcatctgctcccactgtaaccttagtgcagctgcccaatgggcagacagtccaggtccatggggtcatccaggcggcccagccatcagttattcagtctccacaagtccaaacagttcagtcttcctgtaaggacttaaaaagacttttctccggaactcagatttcaactattgcagaaagtgaagattcacaggagtctgtggatagtgtaactgattcccaaaaacgaagggaaatcctttcaaggaggccttcctacaggaaaattttgaatgacttatcttctgatgcaccaggggtgccaaggattgaagaagaaaaatcagaagaagagacttcagcccctgccatcaccactgtaacagtgccaaccccgatttaccaaactagcagtgggcagtatattgccattacccagggaggagcaatacagctggctaacaatggtaccgatggggtacagggcctgcagacattaaccatgaccaatgcagctgccactcagccgggtactaccattctacaatatgcacagaccactgatggacagcagattctagtgcccagcaaccaagttgttgttcaagctgcctctggtgatgtacaaacataccagattcgcacagcacccactagcaccattgcccctggagttgttatggcgtcctccccagcacttcctacacagcctgctgaagaagcagcacgaaagagagaggttcgtctaatgaagaacagggaagcagcaagagaatgtcgtagaaagaagaaagaatatgtgaaatgtttagagaacagagtggcagtgcttgaaaaccaaaacaaaacattgattgaggagctaaaagcacttaaggacctttactgccacaagtcagattaatttgggatttaaattttcacctatgtggtggaaaatggactggattggccacaaccagaa

SEQ ID NO: 62 - DNA, 3616 bp, rattus norvegicus
ccagcccggagcgacagccgcgggcgctccgcaacaccaggtgaacagccatggcgggctggattcaggcccagcagctgcagggagacgctctgcgtcagatgcaagtgttatatgggcaacatttccccatcgaggtccggcactacctggcccagtggatcgagagccagccgtgggatgctattgacttggacaatccccaggaccgaggccaggccacccagctcctggagggcctggtgcaggagctgcagaagaaggcagaacaccaggtgggggaagatggctttgtgctgaagatcaagctggggcactatgccacgcagctccagaacacgtacgaccgctgtcccatggagctggttcgctgtatccgtcacattctgtacaatgaacagaggctggtccgagaagccaacaattgtagctcccctgctggtgtcctggttgacgccatgtcccagaagcaccttcagatcaaccaaacgtttgaggagctgcgcctgatcacacaggacacggagagcgagctgaagaagctgcagcagacccaagagtacttcatcatccagtaccaggagagcctgcggatccaagctcagtttggccagctggcccagctgaacccccaggagcgcatgagcagggagacagccctccagcagaagcaagtgtccctggagacctggctgcagcgagaggcacagacactgcagcagtaccgcgtggagctggttgaaaagcaccagaagaccctgcagttgttgaggaagcagcagaccatcatcctggacgacgagctgatccagtggaagcgggggcagcagctggccgggaacgggggtccccccgagggcagcctggacgtgctgcagtcctggtgtgagaagctggccgagatcatctggcagaaccggcagcagatccgcagggctgagcacctgtgccagcagctgcccatccctggccccgtggaggagatgttggccgaggtcaacgccaccatcacagacatcatctcagccctggtcaccagcacgttcatcatcgagaagcagcctcctcaggtcctgaagacccagaccaagttcgcggccaccgtgcgcctgctggtgggcgggaagctgaacgtgcacatgaaccctccgcaggtgaaggcgaccatcatcagcgagcagcaggccaagtccctgctcaagaacgagaacacccgcaatgattacagtggcgagatcctgaacaactgctgcgtcatggagtaccaccaggccacaggcaccctcagtgcccacttcagaaacatgtcactgaaaagaatcaagcgtgctgacagacgcggtgccgagtctgtgacggaggagaagttcacggtgctgtttgagtctcagttcagcgttggcagcaacgagctggtgttccaggtgaagaccctgtccctccctgtggtcgtcattgttcatggcagccaggaccacaatgctactgccaccgtgctgtgggacaatgcctttgctgagccgggcagggtgccatttgccgtgcctgacaaggtgctgtggccgcagctgtgtgaggcgctcaacatgaagttcaaggctgaggtgcagagcaaccggggcttgaccaaggagaacctcgtgttcctggcacagaaactgttcaacaggaggagcaaccacctcgaggactacaacagcatgtccgtgtcctggtcccagttcaaccgggagaacttgcccggctggaactataccttctggcagtggttcgacggggtgatggaggtgctgaagaagcatcataagccccactggaatgatggggccatcttgggtttcgtgaacaagcagcaggcccatgacctgctcatcaacaagccagatgggacctttctgctgcgattcagcgactcggaaatcgggggcatcaccattgcttggaagtttgactccccggaccgaaacctgtggaatctgaagccattcaccactcgggatttctccattcggtccctggcggaccgcctgggggatctgaattacctcatctacgtgttcccagaccgacccaaggatgaggtctttgccaagtactacactcctgtacttgcaaaagcagtcgacggatacgtgaagccacagatcaagcaagtggtccctgagtttgtcaatgcttctgcagatgctggagccaacgccacctacatggaccaggctccttccccagtggtgtgccctcaggctcactacaacatgtacccaccgaaccccgaccctgtccttgaccaagatggtgaatttgacctggatgagaccatggatgttgccaggcatgtggaggaacttttacgccggcccatggacagtcttgacccccgcctttccccacctgctggcctcttcacttctgccagaagctccttgtcctgaacgctggactccatgcttctcttggaaaagcaccttcagtgtgaagaacccgtgtcagttgtatccctgttcataccagtggctttgcaccttcgcagttccgccttgcccacagtgcggggtctggctaaagtagtggtggccttttcactcagacctcaagggtcccctggacctctcctctaagagctgaacctgtcattgctgacaaacctatttctccagtgcccttcacctgccaggggccactgagtgaaatgattctagaaaaggaagggaggcaagtttatgcaactaaagttggagttttactcctgagctagaagcttcctccggactggtgtgcgtgcacctgtcctcgcacatgtggtagcctaactgatgaacccctctaaagggagattggggctaaccttagtaaccctttatacgttagtgaatatgggctgcggattttgtgaccgctcacaatgtgcataagctgttcccaccaacacaggcggctgccaaagctgactgtgtcagttctcaaaccagcgtggacatgggattcgggtctaaggcctcatctgcatcccttcttccccatccgatgtcctcacccttccatatagattcatccttgctttttgttttgcttttttgagacagggtctctccatatatcccaggctgtccatgaatgatcctcctgcctcagcttctggagtgctaggattataggtttgcatgaccacacctgcctcagcctttcttcctttcctgcttgcaaccttgcttgttatcagaaaggaggggaatactgggtgcctgggattaggagacctagggcgaaaaccttgtagcccacgaaaaggaacagagctgccagttactgcaggcaacacagtggtatgtgaggggggaggctcaacctggtccactctgctgggaggtaggacccggaacaggccctgcctgtgcaacatgggtgtggtctaggaaggcctctcgcaggcagctcttcccttggaggctgtgatagagcggccaggactagctgactaggagtcctggcttccaacctgagggaagcagaggatgcacagagggatggtttttatattttgtacatagaactcacatttatggaaacagtgtttttgaataaaatttctgttgggtttgtttttgcaaaaaaaaaa

SEQ ID NO: 63 - DNA, 1527 bp, rattus norvegicus
atggacaactaccccaaactggaggagatgatgctgctgagcaacggggctccccagttcctcggtgctgccggaaccccagagggcagcggcggcaataacagcagcagcagcagcagcagcagcagcgggggcggtggtgggggcggcagcaacagcggcagcagcgctttcaatcctcaaggggagccgagcgaacaaccctacgagcacctgaccacagagtccttttctgacatcgctctgaataacgagaaggcgctggtggagacaagttatcccagccaaactacccggttgcctcccatcacctatactggccgcttctccctggagcctgcacccaacagtggcaacactttgtggcctgaaccccttttcagcctagtcagtggccttgtgagcatgaccaaccctccaacctcttcatcctcagcgccttctccagctgcttcatcgtcttcctctgcctcccagagcccacccctgagctgtgccgtgccgtccaacgacagcagtcccatttactcagctgcacccacctttcctactcccaacactgacatttttcctgagccccaaagccaggcctttcctggctctgcaggcacagccttgcagtacccgcctcctgcctaccctgccaccaagggtggtttccaggttcccatgatccctgactatctgtttccacaacaacagggagacctgagcctgggcaccccagaccagaagcccttccagggtctggagaaccgtacccagcagccttcgctcactccactatccactatcaaagccttcgccactcagtcgggctcccaggacttaaaggctcttaataacacctaccagtcccaactcatcaaacccagccgcatgcgcaagtaccccaaccggcccagcaagacacccccccatgaacgcccgtatgcttgccctgttgagtcctgcgatcgccgcttttctcgctcggatgagcttacacgccacatccgcatccatacaggccagaagcccttccagtgtcgaatctgcatgcgtaatttcagtcgtagtgaccaccttaccacccacatccgcacccacacaggcgagaagccttttgcctgtgacatttgtgggagaaagtttgccaggagtgatgaacgcaagaggcataccaaaatccacttaagacagaaggacaagaaagcagacaaaagtgtcgtggcctcctcagctgcctcttccctctcttcctacccatccccagtggctacctcctacccatcccccgccaccacctcatttccatccccagtgcccacctcttactcctctccgggctcctctacctacccgtctcctgcacacagtggcttcccatcgccctcggtggccaccacctatgcctccgtcccacctgctttccctgcccaggtcagcaccttccagtctgcaggggtcagcaactccttcagcacctcaacgggtctttcagacatgacagcaaccttttctcctaggacaattgaaatttgctaa